SEQUENCE LISTING (113 sequences)

SEQ ID NO: 1 - protein, 433 aa, Artificial Sequence
  Synthesized: native MTH1p
MFVSPPPATSKNQVLQRRPLESTNSNHGFASSLQAIPENTMSGSDNASFQSLPLSMSSSQSTTSSRRENFVNAPPEYTDRARDEIKKRLLASSPSRRSHHSSSMHSASRRSSVAESGSLLSDNASSYQSSIFSAPSTVHTQLTNDSSFSEFPNHKLITRVSLDEALPKTFYDMYSPDILLADPSNILCNGRPKFTKRELLDWDLNDIRSLLIVEKLRPEWGNQLPEVITVGDNMPQFRLQLLPLYSSDETIIATLVHSDLYMEANLDYEFKLTSAKYTVATARKRHEHITGRNEAVMNLSKPEWRNIIENYLLNIAVEAQCRFDFKQRCSEYKKWKLQQSNLKRPDMPPPSIIPRKNSTETKSLLKKALLKNIQLKNPNNNLDELMMRSSAATNQQGKNKVSLSKEEKATIWSQCQAQVYQRLGLDWQPDSVS

SEQ ID NO: 2 - protein, 358 aa, Artificial Sequence
  Synthesized: Truncated MTH1p
MFVSPPPATSKNQVLQRRPLESTNSNHGFASSLQAIPENTMSGSDNASFQSLPLSMFSAPSTVHTQLTNDSSFSEFPNHKLITRVSLDEALPKTFYDMYSPDILLADPSNILCNGRPKFTKRELLDWDLNDIRSLLIVEKLRPEWGNQLPEVITVGDNMPQFRLQLLPLYSSDETIIATLVHSDLYMEANLDYEFKLTSAKYTVATARKRHEHITGRNEAVMNLSKPEWRNIIENYLLNIAVEAQCRFDFKQRCSEYKKWKLQQSNLKRPDMPPPSIIPRKNSTETKSLLKKALLKNIQLKNPNNNLDELMMRSSAATNQQGKNKVSLSKEEKATIWSQCQAQVYQRLGLDWQPDSVS

SEQ ID NO: 3 - protein, 531 aa, Artificial Sequence
  Synthesized: Modified Acetate CoA-transferase YdiF
MKPVKPPRINGRVPVLSAQEAVNYIPDEATLCVLGAGGGILEATTLITALADKYKQTQTPRNLSIISPTGLGDRADRGISPLAQEGLVKWALCGHWGQSPRISELAEQNKIIAYNYPQGVLTQTLRAAAAHQPGIISDIGIGTFVDPRQQGGKLNEVTKEDLIKLVEFDNKEYLYYKAIAPDIAFIRATTCDSEGYATFEDEVMYLDALVIAQAVHNNGGIVMMQVQKMVKKATLHPKSVRIPGYLVDIVVVDPDQTQLYGGAPVNRFISGDFTLDDSTKLSLPLNQRKLVARRALFEMRKGAVGNVGVGIADGIGLVAREEGCADDFILTVDTGPIGGITSQGIAFGANVNTRAILDMTSQFDFYHGGGLDVCYLSFAEVDQHGNVGVHKFNGKIMGTGGFIDISATSKKIIFCGTLTAGSLKTEITDGKLNIVQEGRVKKFIRELPEITFSGKIALERGLDVRYITERAVFTLKEDGLHLIEIAPGVDLQKDILDKMDFTPVISPELKLMDERLFIDAAMGFVLPEAAH

SEQ ID NO: 4 - DNA, 1818 bp, Artificial Sequence
  Synthesized: Linear cassette for the disruption of PDC1 with an URA3 marker
tatcataactaatatttcttgagataagcacactgcacccataccttccttaaaaacgtagcttccagtttttggtggttccggcttccttcccgattccgcccgctaaacgcatatttttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccctttttctgttagacggtgtcttgatctacttgctatcgttcaacaccaccttattttctaactattttttttttagctcatttgaatcagcttatggtgatggcacatttttgcataaacctagctgtcctcgttgaacataggaaaaaaaaatatataaacaaggctctttcactctccttgcaatcagatttgggtttgttccctttattttcatatttcttgtcatattcctttctcaattattattttctactcataacctcacgcaaaataacacagtcaaatcaatcaaaagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgcgatttaatctctaattattagttaaagttttataagcatt

SEQ ID NO: 5 - DNA, 1467 bp, Artificial Sequence
  Synthesized: Linear cassette for the deletion of PDC6 with URA3 marker
cctagatcgatttgattacaggataagggatatggtgcgattcgcgagcttttaccaatatgtataaaaggcggctgtttgaagccattctatcttaatcttgtgctattgcagtcctcttttatatacagtataaataaaaaacccacgtaatatagcaaaaacatattgccaacaaaagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgccattagtagtgtactcaaacgaattattgttgcaaataaataaacttacacagtttgaatacataaatcaatcagacaaataaatacatcggttcaaattatactaaatctaaatactacgttatcgccgtgaattacgcaattcgcatgttacgtactgcgcgtctcttgttgaata

SEQ ID NO: 6 - DNA, 3642 bp, Artificial Sequence
  Synthesized: Linear cassette for expression of secondary alcohol dehydrogenase from Clostridium beijerinckii controlled by the TEF1 promoter and the PGK1 terminator
gcacggcggttcgattcaattccgccgctccgagcgatgctccgcaaaactcagtaataagctttctgatggttcaccccttttttagcacgcggggtgtaactcaacagaaaaatgtgccatagaacaagactaggcaaaagccaaggagcgtttgccatgaacttccacaatctacgtatggtcatttcttcttcagattccctcatggagaaagtgcggcagatgtatatgacagagtcgccagtttccaagagactttattcaggcacttccatgataggcaagagagaagacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggagacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatgaaaggttttgctatgttgggtattaataagttgggttggatcgaaaaggaaagaccagttgctggttcttacgatgctatagttagaccattggctgtttctccatgtacttctgatatacatacagtctttgaaggtgcattgggtgatagaaagaatatgatcttgggtcatgaagcagttggtgaagttgttgaagttggttctgaggttaaagactttaagcctggtgatagagttattgttccatgtactacaccagactggagatctttggaagttcaagctggttttcagcagcactctaatggtatgttagctggttggaaattctctaattttaaggacggtgttttcggtgaatattttcacgttaatgacgcagacatgaatttggctatattgcctaaagatatgccattggaaaacgcagttatgatcactgatatgatgactactggtttccacggtgctgaattagctgatatacaaatgggttcttctgttgttgttatcggtataggtgctgttggtttgatgggtattgcaggtgctaagttgagaggtgcaggtagaattatcggtgttggttctagaccaatctgcgttgaagcagctaagttttatggtgcaacagacatcttaaactacaagaatggtcatattgttgatcaagttatgaagttgacaaacggtaaaggtgttgacagagtcatcatggctggtggtggttctgagacattgtctcaggctgtctctatggtcaagccaggtggtatcatctctaacatcaactaccacggttctggtgatgccttgttgatcccaagggtcgagtggggttgcggtatggctcacaagaccatcaagggtggattgtgccctggtggtaggttgagagctgagatgttgagagacatggttgtctacaatagagttgatttgtctaagttagttactcatgtttaccacggtttcgatcatatagaagaagcattattgttaatgaaggataaaccaaaggatttgatcaaggctgttgttatattgtaatgaattgaattgaattgaaatcgatagatcaatttttttcttttctctttccccatcctttacgctaaaataatagtttattttattttttgaatattttttatttatatacgtatatatagactattatttatcttttaatgaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacacacgactagcgctttcagatattaaaaagtttagatgtaggttttagcggtaacagttatataaatcgtgtttcttctcttgatgaaacaaaaaaatgctagaaaaactttgtcgtttcttacttttggtgcgctttgcagttttcgtggctagacttagaatcatttctc

SEQ ID NO: 7 - DNA, 4190 bp, Artificial Sequence
  Synthesized: Linear cassette comprising truncated MTH1 gene with its original regulatory elements and a KanMX4 marker gene (flanked by loxp regions).
tcaacaaaggccaaggaaataaagcaaataacaataacaccattattttaattttttttctattactgtcgctaacacctgtatggttgcaaccaggtgagaatccttctgatgcatactttatgcgtttatgctacgctgcaggtcgacaacccttaatataacttcgtataatgtatgctatacgaagttattaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatataacttcgtataatgtatgctatacgaagttattaggtgattacgttggatgtctgcgattggtgatgctaagaactaaaggtatttcgtcatcgtatggtttgatcttcgctacccttgactctttactgcgataacggtaccacgttcccataaaattcttattactaaatctctataaagctgcaacagctagtttgtatatataaatcattggcgcttcttcctcaattccttgggaatttggagctcctctattaaaactgatgtccatgggatagtattgatacaatcgaagagtctccaaaaaaaccatcgggaaggtttctttttagtatcttcaggatactgaatccggctgccaatccaatcctaatctttggtaaacttgtgcctgacattgcgaccatatcgtagccttttcttctttagataagctgactttgttttttccctgttgatttgttgcggcgcttgatctcatcatcaattcatcaaggttattattggggtttttcaactgaatgttcttcaataaagcctttttcagaagcgattttgtttctgtgctgtttttccgcggtattatgcttggtgggggcatgtccggtctttttaagttggactgttgtaacttccatttcttatattcggagcatctttgtttgaaatcaaacctgcattgtgcctctactgctatatttaagaggtaattttcgatgatatttctccattccggtttcgacaaattcatgacggcttcatttctaccagttatatgctcatgtctttttctagcggtcgctactgtatatttggcgctggttagtttgaattcataatctaagttagcctccatgtacagatccgaatggactaacgttgcgattatggtctcatcgctagaatatagtggtaataattgtaacctaaactggggcatattatcacccaccgttattacttccggtagttgattaccccattcgggccttaacttctcgactatcaataacgatcttatatcgtttaaatcccaatccaataactctctcttggtaaacttgggacgcccgttacagagaatgttggatgggtctgctaatagaatatctggcgaatacatgtcataaaacgttttgggtaatgcttcatccaggctcactctcgtgattaacttgtggttaggaaattcggagaacgaagagtcattagttagttgcgtgtgcacagtagagggggcagaaaatatcattgatagtggcaaactttgaaaagaagcattatcactgccactcatcgtgttttccggaatggcctgtagggagcttgcaaacccatgattactgttagtcgattctaatggacgtcgttgtaaaacttggtttttcgaagttgctggtggtggtgaaacaaacattcctttgagtgtgtgtactctatgcgttcgaataaaattctctttgtattgctgcttgtggtttcgctattgagaactttcaatatacgttaaaaaatacttggagtgaaatgaacaaatctttttgtatgtttccttaagttattaaaaaactccatgctgttttttaataaaaaaagaagcagttacaggcgcattacaaagtttgagaagaaaggaaaaatgtcatttggttatataaattttttttcttaatggagattgaggcagcaataaaatgtgggatgaaaaaaaaaaaaaaaggcacttgaataaggggtagaatggcagatattccatgtctacggtagtttcactggagaaatggactcgtattttctctcacgatgaatttttccacaaaattataagatgaggaaatgtgtggggcagtcatcctaacaaaactcgcacggactccatttccccggacctttttcccgcttgttctagggaataatttcggaataattttagggacttcatagtatgactgggtttcaaggaacggaaaaactggttagtaccgcgttattctgccatttgctgatcgttttagtggaggagcggaggagggggcggggtgataactttttttccgctttacaatagagaaaagtccaagtaagtctcggcgtgagatggccttgatgttattagtaccggcggctccctaatagtttactaatacgcataccttatgtcatatttttccatcaaatattgatcagtagaaaaccttggcaatgtacatagagctacacttttttggatatggagtattagagaaaactgggtttggtgggcgaagagagatgttccctacctcctcaaatcagatccactagtggcctatgcggccgcggatctgccggtctccctatagtgagtcgtattaatttcgataagccaggttaacctgcattaatgaatcggccaacgcgcggggagaggcggtttgcgtattgggcgctctgtataataaatttctgatttggtttaaaatatcaactattttttttcacatatgttcttgtaattacttttctgtcctgtcttccag

SEQ ID NO: 8 - DNA, 4348 bp, Artificial Sequence
  Synthesized: Linear cassette for the expression of the gene atoA from E. coli controlled by the TEF1 promoter and CYC1 terminator and the atoD gene from E. coli controlled by the PGK1 promoter and ADH1 terminator
ctgttattgtctagattttttctcggagatggcgcatctatttgccgtcaaaagatcctctcataccatattaagtaaattgcctccatttctttttcctcgggcagagaaactcgcaggcaacttgctctcgaagtggtcacgtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaaatggatgctaaacagagaattgctagaagagttgctcaagagttaagagatggtgacattgttaatttgggtataggtttacctacaatggttgcaaactacttaccagagggtattcacatcactttacagtctgaaaatggtttcttgggtttaggtccagttactactgcacaccctgacttggttaacgcaggtggtcaaccttgcggtgttttaccaggtgctgcaatgtttgattctgctatgtcttttgctttaattagaggtggtcatatagacgcttgcgttttaggtggtttgcaagttgatgaggaggctaacttagctaactgggttgttccaggtaagatggttccaggtatgggtggtgcaatggatttggttactggttctagaaaagttatcattgcaatggaacattgcgctaaggatggttctgctaaaatcttaagaagatgcactatgccattaacagctcagcatgctgttcacatgttagttacagaattagcagttttcagattcatagatggtaaaatgtggttaacagaaatagcagatggttgtgatttggctactgttagagctaagacagaagcaagatttgaagttgctgctgatttgaacacacaaagaggtgacttataatgaatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggaagaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatgaagactaagttgatgacattacaagatgcaacaggtttctttagagatggtatgacaattatggttggtggttttatgggtataggtactccatctagattagttgaggctttgttagagtctggtgttagagacttaactttaatagctaatgatactgctttcgttgatactggtatcggtccattaattgttaatggtagagttagaaaggttatcgcttctcatattggtacaaatcctgaaactggtagaagaatgatctctggtgaaatggatgttgttttagttcctcaaggtacattaattgagcaaattagatgcggtggtgctggtttgggtggtttcttgactccaacaggtgttggtacagttgttgaagaaggtaagcaaactttaacattggatggtaagacttggttgttagaaagaccattgagagcagatttggcattaattagagcacatagatgcgatactttaggtaatttgacttaccaattatctgcaagaaattttaaccctttgatcgctttagctgcagatataacattagttgagccagacgagttagttgaaactggtgagttacagccagaccacattgttactccaggtgctgttattgaccatattatcgtttctcaagaatctaagtaatgacgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcccctgcataatcggccctcacagagggatcccgttacccatctatgctgaagatttatcatactattcctccgctcgtttcttttttcagtgaggtgtgtcgtgaaagaaaaccc

SEQ ID NO: 9 - DNA, 5034 bp, Artificial Sequence
  Synthesized: Linear cassette for the expression of the thiolase gene from C. acetobutylicum controlled by the ADH1 promoter and TEF1 terminator and the acetoacetate decarboxylase gene from C. beijerinckii
acgactgtttaaacattcatatttcgcatgagccaacatacagttgctgagtaatcttcattgcgcttatttatcggcattgagattgtaaaggaagtaaaacgcatttttgcagatctgttctcttatgtatttttaatcgtccttgtatggaagtatcaaaggggacgttcttcacctccttggaaaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgttggaatctgaagtttctaaacaaattacaactccattagctgctccagctttcccaagaggtccatacagatttcataacagagagtacttgaacattatctatagaactgacttagacgcattaagaaagatcgttccagaaccattagagttggatagagcatacgttagatttgaaatgatggctatgcctgatactacaggtttgggttcttacactgaatgcggtcaggcaataccagttaagtacaatggtgttaagggtgattacttgcacatgatgtacttagataatgagcctgcaatagcagttggtagagagtcttctgcatatccaaagaaattaggttatccaaaattattcgttgactctgatactttagttggtacattaaaatacggtactttgccagttgcaacagctactatgggttacaaacatgaacctttggacttaaaggaggcttatgctcaaattgcaagaccaaacttcatgttgaaaatcatccaaggttacgatggtaaaccaagaatttgcgaattgatttgtgctgaaaatactgacattacaatacatggtgcatggactggttctgctagattacaattattttctcacgctttagcacctttagctgacttgccagttttggaaatagtttctgcttctcatattttgactgatttgacattaggtactcctaaagttgttcacgattatttgtctgttaagtaatgagttattactgagtagtatttatttaagtattgtttgtgcacttgcctgcaggccttttgaaaagcaagcataaaagatctaaacataaaatctgtaaaataacaagatgtaaagataatgctaaatcatttggctttttgattgattgtacaggaaaatatacatcgcagggggttgacttggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacaatgaaggaagttgttattgcatctgcagttagaacagctattggttcttatggtaagtctttaaaagacgttcctgctgttgatttgggtgctacagcaattaaggaggcagttaagaaagctggtatcaaacctgaagatgttaatgaagttatattaggtaatgttttacaagctggtttaggtcaaaatccagcaagacaagcatctttcaaagctggtttaccagttgagatccctgctatgacaattaacaaggtttgtggttctggtttgagaacagtttctttagctgctcagattattaaagctggtgatgcagatgttataatagcaggtggtatggaaaatatgtctagagcaccttatttggctaataatgcaagatggggttacagaatgggtaacgcaaagttcgttgatgagatgatcacagacggtttgtgggatgcattcaatgattatcacatgggtattacagcagaaaacattgctgaaagatggaatatatctagagaagaacaggatgaatttgctttagcttctcagaagaaagctgaagaagctattaagtctggtcaattcaaagatgaaattgttccagttgttattaaaggtagaaaaggtgagacagttgttgatactgatgagcatcctagatttggttctacaatcgaaggtttagcaaagttgaagccagctttcaagaaagatggtactgttactgctggtaatgcatctggtttgaatgactgtgctgctgttttggttattatgtctgcagaaaaggctaaggagttgggtgttaaaccattggctaaaatagtttcttatggttctgcaggtgttgaccctgcaataatgggttacggtccattttatgcaacaaaggctgcaattgagaaggcaggttggactgttgatgaattggatttgattgaatctaatgaggctttcgctgcacaatctttagctgttgctaaggacttgaaatttgatatgaataaagttaatgttaacggtggtgcaatcgcattgggtcatccaatcggtgcatctggtgctagaatattagttacattggttcatgctatgcaaaagagagatgctaagaagggtttagcaactttatgtattggtggtggtcaaggtactgcaatattgttagagaagtgctaatgaggagattgataagacttttctagttgcatatcttttatatttaaatcttatctattagttaattttttgtaatttatccttatatatagtctggttattctaaaatatcatttcagtatctaaaaattcccctcttttttcagttatatcttaacaggcgacagtccaaatgttgatttatcccagtccgattcatcagagcttgccttgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgagatgggacgtcagcactgtacttgtttttgcgactagattgtaaatcattctttatttaatctctttctttaactactgcttaaagtataatttggtccgtagtttaataactatactaagcgtaacaatgcatactgacattataagcctgaacattacgagtttaaactgacta

SEQ ID NO: 10 - DNA, 12889 bp, Artificial Sequence
  Synthesized: Plasmid pRS415 backbone expressing tPFLA gene from E. coli and PFLB gene from E. coli
gacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagcgcgcaattaaccctcactaaagggaacaaaagctggagctccaccgcggtggcggccgctctagaactagttgccggaggaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgacttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaaatgtcagttattggtcgcattcactcctttgaatcctgtggaaccgtagacggcccaggtattcgctttatcacctttttccagggctgcctgatgcgctgcctgtattgtcataaccgcgacacctgggacacgcatggcggtaaagaagttaccgttgaagatttgatgaaggaagtggtgacctatcgccactttatgaacgcttccggcggcggcgttaccgcatccggcggtgaagcaatcctgcaagctgagtttgttcgtgactggttccgcgcctgcaaaaaagaaggcattcatacctgtctggacaccaacggttttgttcgtcgttacgatccggtgattgatgaactgctggaagtaaccgacctggtaatgctcgatctcaaacagatgaacgacgagatccaccaaaatctggttggagtttccaaccaccgcacgctggagttcgctaaatatctggcgaacaaaaatgtgaaggtgtggatccgctacgttgttgtcccaggctggtctgacgatgacgattcagcgcatcgcctcggtgaatttacccgtgatatgggcaacgttgagaaaatcgagcttctcccctaccacgagctgggcaaacacaaatgggtggcaatgggtgaagagtacaaactcgacggtgttaaaccaccgaagaaagagactatggaacgcgtgaaaggcattcttgagcagtacggtcataaggtaatgttctaagcttataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccggcatgcgctgcaaggaggatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagacggcctcgtttctttttcttcgtcgaaaaaggcaataaaaatttttatccgtttctttttcttgaaaatttttttttttgatttttttctctttcgatgacctcccattgatatttaagttaataaacggtcttgaatttctcaagtttcagtttcatttttcttgttctattacaactttttttacttcttgctcattagaaagaaagcatagcaatctaatctaagttttaattacaaaaatgtccgagcttaatgaaaagttagccacagcctgggaaggttttaccaaaggtgactggcagaatgaagtaaacgtccgtgacttcattcagaaaaactacactccgtacgagggtgacgagtccttcctggctggcgctactgaagcgaccaccaccctgtgggacaaagtaatggaaggcgttaaactggaaaaccgcactcacgcgccagttgactttgacaccgctgttgcttccaccatcacctctcacgacgctggctacatcaacaagcagcttgagaaaatcgttggtctgcagactgaagctccgctgaaacgtgctcttatcccgttcggtggtatcaaaatgatcgaaggttcctgcaaagcgtacaaccgcgaactggatccgatgatcaaaaaaatcttcactgaataccgtaaaactcacaaccagggcgtgttcgacgtttacactccggacatcctgcgttgccgtaaatctggtgttctgaccggtctgccagatgcatatggccgtggccgtatcatcggtgactaccgtcgcgttgcgctgtacggtatcgactacctgatgaaagacaaactggcacagttcacttctctgcaggctgatctggaaaacggcgtaaacctggaacagactatccgtctgcgcgaagaaatcgctgaacagcaccgcgctctgggtcagatgaaagaaatggctgcgaaatacggctacgacatctctggtccggctaccaacgctcaggaagctatccagtggacttacttcggctacctggctgctgttaagtctcagaacggtgctgcaatgtccttcggtcgtacctccaccttcctggatgtgtacatcgaacgtgacctgaaagctggcaagatcaccgaacaagaagcgcaggaaatggttgaccacctggtcatgaaactgcgtatggttcgcttcctgcgtactccggaatacgatgaactgttctctggcgacccgatctgggcaaccgaatctatcggtggtatgggcctcgacggtcgtaccctggttaccaaaaacagcttccgtttcctgaacaccctgtacaccatgggtccgtctccggaaccgaacatgaccattctgtggtctgaaaaactgccgctgaacttcaagaaattcgccgctaaagtgtccatcgacacctcttctctgcagtatgagaacgatgacctgatgcgtccggacttcaacaacgatgactacgctattgcttgctgcgtaagcccgatgatcgttggtaaacaaatgcagttcttcggtgcgcgtgcaaacctggcgaaaaccatgctgtacgcaatcaacggcggcgttgacgaaaaactgaaaatgcaggttggtccgaagtctgaaccgatcaaaggcgatgtcctgaactatgatgaagtgatggagcgcatggatcacttcatggactggctggctaaacagtacatcactgcactgaacatcatccactacatgcacgacaagtacagctacgaagcctctctgatggcgctgcacgaccgtgacgttatccgcaccatggcgtgtggtatcgctggtctgtccgttgctgctgactccctgtctgcaatcaaatatgcgaaagttaaaccgattcgtgacgaggacggtctggctatcgacttcgaaatcgaaggcgaatacccgcagtttggtaacaatgatccgcgtgtagatgacctggctgttgacctggtagaacgtttcatgaagaaaattcagaaactgcacacctaccgtgacgctatcccgactcagtctgttctgaccatcacttctaacgttgtgtatggtaagaaaacgggtaacaccccagacggtcgtcgtgctggcgcgccgttcggaccgggtgctaacccgatgcacggtcgtgaccagaaaggtgcagtagcctctctgacttccgttgctaaactgccgtttgcttacgctaaagatggtatctcctacaccttctctatcgttccgaacgcactgggtaaagacgacgaagttcgtaagaccaacctggctggtctgatggatggttacttccaccacgaagcatccatcgaaggtggtcagcacctgaacgttaacgtgatgaaccgtgaaatgctgctcgacgcgatggaaaacccggaaaaatatccgcagctgaccatccgtgtatctggctacgcagtacgtttcaactcgctgactaaagaacagcagcaggacgttattactcgtaccttcactcaatctatgtaataagcttgtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgcgctactaggagaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaaatgccacattcctacgattacgatgccatagtaataggttccggccccggcggcgaaggcgctgcaatgggcctggttaagcaaggtgcgcgcgtcgcagttatcgagcgttatcaaaatgttggcggcggttgcacccactggggcaccatcccgtcgaaagctctccgtcacgccgtcagccgcattatagaattcaatcaaaacccactttacagcgaccattcccgactgctccgctcttcttttgccgatatccttaaccatgccgataacgtgattaatcaacaaacgcgcatgcgtcagggattttacgaacgtaatcactgtgaaatattgcagggaaacgctcgctttgttgacgagcatacgttggcgctggattgcccggacggcagcgttgaaacactaaccgctgaaaaatttgttattgcctgcggctctcgtccatatcatccaacagatgttgatttcacccatccacgcatttacgacagcgactcaattctcagcatgcaccacgaaccgcgccatgtacttatctatggtgctggagtgatcggctgtgaatatgcgtcgatcttccgcggtatggatgtaaaagtggatctgatcaacacccgcgatcgcctgctggcatttctcgatcaagagatgtcagattctctctcctatcacttctggaacagtggcgtagtgattcgtcacaacgaagagtacgagaagatcgaaggctgtgacgatggtgtgatcatgcatctgaagtcgggtaaaaaactgaaagctgactgcctgctctatgccaacggtcgcaccggtaataccgattcgctggcgttacagaacattgggctagaaactgacagccgcggacagctgaaggtcaacagcatgtatcagaccgcacagccacacgtttacgcggtgggcgacgtgattggttatccgagcctggcgtcggcggcctatgaccaggggcgcattgccgcgcaggcgctggtaaaaggcgaagccaccgcacatctgattgaagatatccctaccggtatttacaccatcccggaaatcagctctgtgggcaaaaccgaacagcagctgaccgcaatgaaagtgccatatgaagtgggccgcgcccagtttaaacatctggcacgcgcacaaatcgtcggcatgaacgtgggcacgctgaaaattttgttccatcgggaaacaaaagagattctgggtattcactgctttggcgagcgcgctgccgaaattattcatatcggtcaggcgattatggaacagaaaggtggcggcaacactattgagtacttcgtcaacaccacctttaactacccgacgatggcggaagcctatcgggtagctgcgttaaacggtttaaaccgcctgttttaagcttataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccggcatgcgctttacctcgaggtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatcgacggtcgaggagaacttctagtatatccacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacaacaaataaaaaacactcaatgacctgaccatttgatggagtttaagtcaataccttcttgaaccatttcccataatggtgaaagttccctcaagaattttactctgtcagaaacggccttacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 11 - DNA, 13170 bp, Artificial Sequence
  Synthesized: Plasmid pRS415 backbone expressing three copies of the Bacillus subtilis mgsA,and the yqhD gene from E. coli  and the GRE2 gene from S. cerevisiae and the udhA gene from E. coli
tgccggaggaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctttcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaaatgaacaattttaatttgcatactccaactagaatattatttggaaaaggtgcaattgcaggtttaagggaacaaataccacatgatgcaagggtattaatcacatacggtggtggttctgtcaagaaaactggtgtattggatcaagtattggatgctttaaagggtatggatgtcttggaatttggaggaatcgaaccaaaccctgcttacgagactttaatgaatgctgtcaaattggtcagagaacaaaaggtaacattcttattggctgttggaggtggatcagtattagatggtacaaagttcattgctgctgcagcaaattatccagaaaacattgatccatggcatatattgcaaactggtggtaaggaaataaagtcagctatcccaatgggatgtgttttgacattgcctgcaacaggatcagaatcaaacgctgaagcagtcatctcaagaaagactacaggtgacaaacaggcattccattctgcccatgtccaacctgtatttgctgttttagaccctgtatacacttacacattaccaccaaggcaagtcgcaaatggagttgtcgatgcctttgttcacactgtagaacagtacgtcaccaaaccagtcgatgcaaagatccaggacaggtttgcagaaggtattttattgacattaatcgaagatggaccaaaagcattgaaagagccagagaactatgacgttagggcaaatgttatgtgggctgctacccaggcattgaacggtttaattggtgcaggagttccacaagattgggctacacacatgttgggtcacgagttgaccgccatgcacggtttggaccatgcacagactttagccattgttttgcctgccttatggaacgagaaaagagatactaagagggctaagttattacaatacgctgaaagggtttggaatatcaccgagggatctgatgatgaaaggattgatgccgctattgcagccactagaaacttctttgaacaattaggtgttccaactcacttgtctgactatggtttagatggatcatctattccagctttgttgaagaaattggaagagcacggtatgacccagttgggtgagaatcatgatataaccttagatgtatctaggagaatctacgaggctgctagataatgagcttataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccggcatgcgctgcaaggaggaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctttcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaaatgtctgtctttgtatcaggagctaacggttttatcgcacaacacatagttgatttgttgttgaaagaggactacaaggtaatcggttcagctagatcacaagagaaagctgaaaacttaaccgaggcttttggtaacaatccaaagttctcaatggaagttgtccctgacatttctaagttggacgcatttgatcacgttttccaaaagcatggtaaagacattaaaattgttttacacactgcttcaccattttgtttcgacattactgactctgaaagggatttgttaatcccagcagtcaacggtgtcaagggtatattacactcaataaagaaatatgcagccgattcagtcgaaagggttgttttgacttcatcttacgctgctgtatttgacatggccaaggagaatgacaagtctttaacattcaacgaagaatcatggaatcctgctacctgggagtcttgtcaatcagatccagttaatgcatattgtggttcaaagaagtttgccgaaaaggccgcttgggagttcttagaagagaatagagactctgttaagtttgaattaacagccgtcaatccagtatacgtctttggtccacagatgttcgacaaagacgtcaagaaacacttgaacacctcttgcgagttggttaattctttgatgcacttatctccagaggataaaatacctgaattattcggtggttacatcgatgtcagggatgtcgcaaaagcccacttggtcgcattccaaaagagagaaacaataggtcaaagattgatcgtttctgaggctaggttcaccatgcaagatgtcttagacattttgaatgaagattttcctgtcttaaagggaaatatcccagtaggtaagccaggttcaggagctacccacaatactttgggagctaccttagacaacaagaaatcaaagaagttattaggttttaagttcagaaacttgaaggaaactattgatgataccgcatctcagattttaaagttcgaaggtagaatctaatgagcttatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagcgctactaggagtctacgtatggtcatttcttcttcagattccctcatggagaaagtgcggcagatgtatatgacagagtcgccagtttccaagagactttattcaggcacttccatgataggcaagagagatgacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggacacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaaatgatgaagatagcattaattgcacatgataagaagaagcaagatatggttcaattcacaactgcttacagagacatcttaaagaatcatgacttgtacgctactggtactacaggtttaaagatacatgaggcaactggtttgcagattgagagatttcaatctggtccattgggtggtgaccaacaaataggtgcattgatcgcagcaaacgctttagacttggttatcttcttaagagatccattgactgcacaacctcacgaaccagatgtttctgctttaataagattatgtgatgtttactctattccattggctactaatatgggtactgctgaaatattagttagaacattagacgagggtgttttcgagtttagagacttgttgagaggtgaagaacctaatgtttaatgagcttgtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgcgctttacggagtctacgtatggtcatttcttcttcagattccctcatggagaaagtgcggcagatgtatatgacagagtcgccagtttccaagagactttattcaggcacttccatgataggcaagagagatgacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggacacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaaatgatgaagatagcattaattgcacatgataagaagaagcaagatatggttcaattcacaactgcttacagagacatcttaaagaatcatgacttgtacgctactggtactacaggtttaaagatacatgaggcaactggtttgcagattgagagatttcaatctggtccattgggtggtgaccaacaaataggtgcattgatcgcagcaaacgctttagacttggttatcttcttaagagatccattgactgcacaacctcacgaaccagatgtttctgctttaataagattatgtgatgtttactctattccattggctactaatatgggtactgctgaaatattagttagaacattagacgagggtgttttcgagtttagagacttgttgagaggtgaagaacctaatgtttaatgagcttgtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgcgctcagaggagtctacgtatggtcatttcttcttcagattccctcatggagaaagtgcggcagatgtatatgacagagtcgccagtttccaagagactttattcaggcacttccatgataggcaagagagatgacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggacacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaaatgatgaagatagcattaattgcacatgataagaagaagcaagatatggttcaattcacaactgcttacagagacatcttaaagaatcatgacttgtacgctactggtactacaggtttaaagatacatgaggcaactggtttgcagattgagagatttcaatctggtccattgggtggtgaccaacaaataggtgcattgatcgcagcaaacgctttagacttggttatcttcttaagagatccattgactgcacaacctcacgaaccagatgtttctgctttaataagattatgtgatgtttactctattccattggctactaatatgggtactgctgaaatattagttagaacattagacgagggtgttttcgagtttagagacttgttgagaggtgaagaacctaatgtttaatgagcttgtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgcgcttgtgggagaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaaatgccacattcctacgattacgatgccatagtaataggttccggccccggcggcgaaggcgctgcaatgggcctggttaagcaaggtgcgcgcgtcgcagttatcgagcgttatcaaaatgttggcggcggttgcacccactggggcaccatcccgtcgaaagctctccgtcacgccgtcagccgcattatagaattcaatcaaaacccactttacagcgaccattcccgactgctccgctcttcttttgccgatatccttaaccatgccgataacgtgattaatcaacaaacgcgcatgcgtcagggattttacgaacgtaatcactgtgaaatattgcagggaaacgctcgctttgttgacgagcatacgttggcgctggattgcccggacggcagcgttgaaacactaaccgctgaaaaatttgttattgcctgcggctctcgtccatatcatccaacagatgttgatttcacccatccacgcatttacgacagcgactcaattctcagcatgcaccacgaaccgcgccatgtacttatctatggtgctggagtgatcggctgtgaatatgcgtcgatcttccgcggtatggatgtaaaagtggatctgatcaacacccgcgatcgcctgctggcatttctcgatcaagagatgtcagattctctctcctatcacttctggaacagtggcgtagtgattcgtcacaacgaagagtacgagaagatcgaaggctgtgacgatggtgtgatcatgcatctgaagtcgggtaaaaaactgaaagctgactgcctgctctatgccaacggtcgcaccggtaataccgattcgctggcgttacagaacattgggctagaaactgacagccgcggacagctgaaggtcaacagcatgtatcagaccgcacagccacacgtttacgcggtgggcgacgtgattggttatccgagcctggcgtcggcggcctatgaccaggggcgcattgccgcgcaggcgctggtaaaaggcgaagccaccgcacatctgattgaagatatccctaccggtatttacaccatcccggaaatcagctctgtgggcaaaaccgaacagcagctgaccgcaatgaaagtgccatatgaagtgggccgcgcccagtttaaacatctggcacgcgcacaaatcgtcggcatgaacgtgggcacgctgaaaattttgttccatcgggaaacaaaagagattctgggtattcactgctttggcgagcgcgctgccgaaattattcatatcggtcaggcgattatggaacagaaaggtggcggcaacactattgagtacttcgtcaacaccacctttaactacccgacgatggcggaagcctatcgggtagctgcgttaaacggtttaaaccgcctgttttaagcttataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccggcatgcgctgagcccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaactccgtcgaacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcagcttggcccagtgccaagctcaatacgcaaaccgcctctccccgcgcgttggccgattcattaatgcagctggcacgacaggtttcccgactggaaagcgggcagtgagcgcaacgc

SEQ ID NO: 12 - DNA, 2433 bp, Artificial Sequence
  Synthesized: PFLB
atgaccacactgaaactggacacgctcagcgaccgcattaaagcgcacaaaaatgcgctggtgcatattgtgaaaccgccagtctgtaccgagcgcgcgcagcactataccgagatgtatcaacaacatctcgataagccgatcccggtacgtcgcgcgctggcactggcgcatcacctggcgaatcgcaccatctggatcaaacacgatgagttgatcattggcaaccaggcaagcgaagttcgcgccgcgccgatcttcccggaatatactgtctcgtggatcgaaaaagagattgatgatctggcagatcgtcccggtgctggctttgcggtgagcgaagagaacaaacgcgttctgcatgaagtgtgcccgtggtggcgcggtcagaccgtacaggatcgctgctacggcatgtttaccgatgagcaaaaaggtctgctggcgaccggaatcattaaagcggaaggcaatatgacctccggcgatgcgcacctggcggtgaatttcccgctgctgctggaaaaagggcttgatggtctgcgcgaggaagtagcggaacgtcgctcgcgcatcaacctgacggtgctggaagatttacacggtgagcaattcctgaaagcgattgatatcgtgctggtggcagtcagtgaacacattgaacgtttcgctgccctggcgcgtgaaatggccgcgaccgaaacccgcgaaagccgtcgcgatgaactgctggcgatggcagaaaactgcgatcttatcgcccaccagccgccgcagactttctggcaggcgctgcaactgtgttacttcatccagttgattttgcagatcgaatctaacggtcactcagtatcgtttggtcgtatggaccagtatctctacccgtactatcgccgcgacgttgaactcaaccagacgctggatcgcgaacacgccatcgagatgctgcatagctgctggctgaaactgctggaagtgaacaagatccgctccggctcacactcaaaagcctctgcgggaagtccgctgtatcagaacgtcactattggcgggcaaaatctggttgatggtcaaccaatggacgcggtgaatccactctcttacgcgatcctcgaatcctgcggtcgcctgcgttccactcagcctaacctcagcgtgcgttaccatgcaggaatgagcaacgatttcctcgacgcctgcgtacaggtgatccgttgcggcttcgggatgccggcgttcaacaacgacgaaatcgtgatcccggaatttattaaactcggtattgaaccgcaggacgcttatgactacgcagcgattggttgtatagaaaccgccgtcggtggcaaatggggctatcgctgtaccggcatgagctttatcaacttcgcccgcgtgatgctggcggcgctggaaggcgggcatgatgccaccagcggcaaagtgttcctgccacaagaaaaagcgttgtcggcaggtaacttcaacaacttcgatgaagtgatggacgcgtgggatacgcaaatccgttactacacccgcaaatcaatcgaaatcgaatatgtcgtcgacaccatgctggaagagaacgtgcacgatattctctgctcggcgctggtggatgactgtattgagcgagcgaaaagtatcaagcaaggcggcgcgaaatatgactgggtttctggcctgcaggtcggcattgccaacctcggcaacagcctggcggcagtgaagaaactggtgtttgaacaaggtgcgattggtcagcaacagcttgctgccgcactggcagatgacttcgacggcctgactcacgagcagctgcgtcagcggctgattaacggtgcgccgaagtacggcaacgacgatgatactgtcgatacgctgctggctcgcgcttatcagacctatatcgacgaactgaaacagtaccataatccgcgctacggtcgtggtccggttggcggcaactattacgcgggtacgtcatcaatctccgctaacgtaccgtttggcgcgcagactatggcaacaccggacgggcgtaaagcccacaccccgctggcagaaggcgcaagcccggcctccggtactgaccatcttggccctactgcggtcattggctcagtgggtaaactgcctacggcagcgattctcggcggcgtgttgctcaaccagaaactgaatccggcaacgctggagaacgaatctgacaagcagaaactgatgatcctgctgcgtaccttctttgaagtgcataaaggctggcatattcagtacaacatcgtttcccgcgaaacgctgctggatgcgaaaaaacatcccgatcagtatcgcgatctggtagtgcgtgtcgcgggctattccgcgttcttcaccgcgctctctccagacgctcaggacgatatcatcgcccgtactgaacatatgctgtaa

SEQ ID NO: 13 - protein, 810 aa, Artificial Sequence
  Synthesized: PFLB Protein P75793
MTTLKLDTLSDRIKAHKNALVHIVKPPVCTERAQHYTEMYQQHLDKPIPVRRALALAHHLANRTIWIKHDELIIGNQASEVRAAPIFPEYTVSWIEKEIDDLADRPGAGFAVSEENKRVLHEVCPWWRGQTVQDRCYGMFTDEQKGLLATGIIKAEGNMTSGDAHLAVNFPLLLEKGLDGLREEVAERRSRINLTVLEDLHGEQFLKAIDIVLVAVSEHIERFAALAREMAATETRESRRDELLAMAENCDLIAHQPPQTFWQALQLCYFIQLILQIESNGHSVSFGRMDQYLYPYYRRDVELNQTLDREHAIEMLHSCWLKLLEVNKIRSGSHSKASAGSPLYQNVTIGGQNLVDGQPMDAVNPLSYAILESCGRLRSTQPNLSVRYHAGMSNDFLDACVQVIRCGFGMPAFNNDEIVIPEFIKLGIEPQDAYDYAAIGCIETAVGGKWGYRCTGMSFINFARVMLAALEGGHDATSGKVFLPQEKALSAGNFNNFDEVMDAWDTQIRYYTRKSIEIEYVVDTMLEENVHDILCSALVDDCIERAKSIKQGGAKYDWVSGLQVGIANLGNSLAAVKKLVFEQGAIGQQQLAAALADDFDGLTHEQLRQRLINGAPKYGNDDDTVDTLLARAYQTYIDELKQYHNPRYGRGPVGGNYYAGTSSISANVPFGAQTMATPDGRKAHTPLAEGASPASGTDHLGPTAVIGSVGKLPTAAILGGVLLNQKLNPATLENESDKQKLMILLRTFFEVHKGWHIQYNIVSRETLLDAKKHPDQYRDLVVRVAGYSAFFTALSPDAQDDIIARTEHML

SEQ ID NO: 14 - DNA, 927 bp, Artificial Sequence
  Synthesized: PFLA
atgcttgaacgaaatagagaggcaactatgattttcaatattcagcgctactcgacccatgatggccccggtatccgcacggtcgtatttcttaaaggctgttcgctgggctgccgctggtgtcagaacccggaaagccgcgcccgcacgcaggatctgctgtatgacgcacgactgtgtctggaaggctgcgagctgtgcgctaaggccgcgccggaagtgattgagcgcgcgctgaatggtttgcttattcatcgggaaaagttaaccccggagcatctgacggcgttaaccgactgctgtccgacacaggcattaaccgtgtgtggtgaagtgaaaagcgttgaggagatcatgacgaccgttctgcgcgataaaccgttttacgatcgcagcggcggcggtttaacgctttcgggtggtgagccctttatgcagccggaaatggcgatggcgctactgcaagccagccacgaggcaggcattcatactgcggtagaaacctgtctgcatgtgccgtggaaatatatcgccccttctctgccctatatcgatctgtttcttgccgatttaaaacacgttgccgacgcgccgtttaaacagtggaccgacggtaacgccgccagagtgctggataacctgaaaaaactcgccgcagcgggcaaaaaaatcattatccgcgtgccgctgattcagggctttaatgccgacgaaacctctgtaaaagccattaccgattttgccgccgacgagctgcacgttggcgaaattcattttctgccctaccacacgctgggcatcaacaaatatcacttacttaatctgccctatgacgccccggaaaaaccgcttgatgcgccagaactgctcgactttgcccagcagtatgcctgccagaaagggttaaccgcgaccttacgaggataa

SEQ ID NO: 15 - protein, 308 aa, Artificial Sequence
  Synthesized: PFLA Protein C4ZXZ6
MLERNREATMIFNIQRYSTHDGPGIRTVVFLKGCSLGCRWCQNPESRARTQDLLYDARLCLEGCELCAKAAPEVIERALNGLLIHREKLTPEHLTALTDCCPTQALTVCGEVKSVEEIMTTVLRDKPFYDRSGGGLTLSGGEPFMQPEMAMALLQASHEAGIHTAVETCLHVPWKYIAPSLPYIDLFLADLKHVADAPFKQWTDGNAARVLDNLKKLAAAGKKIIIRVPLIQGFNADETSVKAITDFAADELHVGEIHFLPYHTLGINKYHLLNLPYDAPEKPLDAPELLDFAQQYACQKGLTATLRG

SEQ ID NO: 16 - DNA, 2415 bp, Artificial Sequence
  Synthesized: PFLB 387233060
atggaaagtttaactttagtcaacaacgctcttgtcaagtcagtttcagttaatgctgttgctgccactaaggttgctggtgttagaatcagcaagccatctcgtgctattcacactactccaatgaccactactagtcttaaggttgctaagaaggctgccttctctcaatctaagacttatgctactgctccatgcattactaatgatgctgctgccaagagtgaaatcgatgttgaaggttggattaagaagcactacactccatatgaaggagatggttctttccttgctggtccaactgaaaagactaagaagctttttgccaaggctgaagaatacttagccaaggaacgtgctaacggtggtttatacgatgttgacccacacaccccatctaccattacttctcacaagccaggttaccttgacaaagaaaatgaagttatctacggttaccaaactgatgttccacttaagagagccattaagccattcggtggtgttaatatggtaaagaacgctcttaaggctgttaacgttccaatggataaggaagttgaacacattttcactgattaccgtaagactcacaacactgctgtattcgatatttactctaaggaaatgagagctggtcgttccaatgctatcatgaccggtttaccagatggttatggtcgtggtcgtattattggtgattaccgtcgtgttgccctttacggtactgaccgtcttattgcccaaaagcaaaaggataaggttgaattacaaaagagacaaatggatgaaccaactatgaaattaattggtgaagttgctgatcaaattaaggctcttaagcaacttactcaaatggccaagtcttacggtattgatattactaagccagctaagaacgccagagaagctactcaattcgtttacttcggttacttaggttctatcaaggaacaagatggtgctgctatgtctcttggtcgtgttgatgccttccttgattgtttcttcgaaaatgatttaaagaatggtgttcttgatgaagcccatgcccaagaaattattgataaccttatcttaaagttacgtttcgctcgtcacttacgtactccagaatacaacgatttattcgctggtgatccaacctgggttactatgtctctcggtggtactggttctgatggtcgtacattagttaccaagacttccttccgtgttcttaacactctttacaacttaggtccagctccagaaccaaacatcactgtcctttggaacaagaaccttccaaagaactttaaggactttgctactaaggtttctattgatacctcttccattcaatacgaatctgatgctcttatgtccgctagattcggtgatgactacggtattgcttgctgtgtctctgccatgagaattggtaaggatatgcaattcttcggtgctcgttgtaaccttgctaagcttatgctttacgtcctcaaccatggtaaggatgaaagaactggtaagcaagttggtccagactttggtccagttccagatggtccaattccattcgactggatgtgggaaacctatgacaaggctatggactggattgccaagctttacgtcaacaccatgaacgttattcacttctgccatgaccaatactgttacgaatcccttcaaatggctcttcatgataccgatgtccgtcgtcttatggccttcggtgttgctggtctttctgttgttgctgattcattctctgctattaagtacgccaaggttactccaatccgtgatccaaagaccggtttaactactgactttaaggttgaaggtgaattcccaaaattcggtaatgatgatgaccgtgtcgatttcttcgctcgtaccgttactgataagcttattaccaagttaagaaaaactccaacttaccgtggtgccactcacactctttccattcttaccattacctctaatgtcgtttacggtaagaagaccggttctactccagatggtcgtaaggctggtcaaccattcgctccaggttgtaacccaatgcacggtcgtgaattctctggtgctgttgcttctctttcttcagtcgctaaggttaactacgactcttgtatggatggtatttctaacaccttctctattgttccaaacaccattggtaagaccttacaagaacgtcaaggtaacctttccggtttattagatggttacttcagcaagggtgctcaccatcttaacgttaacgttcttaagcgtgaaactttagaagatgccatggctcacccagaaaactatccaaaccttactattcgtgtttctggttatgctgttaactttgttaagttaactccagctcaacaaaaggaagtcattgcccgtaccttccacgaaaagatgtaa

SEQ ID NO: 17 - protein, 804 aa, Artificial Sequence
  Synthesized: PFLB  Protein K9LI23
MESLTLVNNALVKSVSVNAVAATKVAGVRISKPSRAIHTTPMTTTSLKVAKKAAFSQSKTYATAPCITNDAAAKSEIDVEGWIKKHYTPYEGDGSFLAGPTEKTKKLFAKAEEYLAKERANGGLYDVDPHTPSTITSHKPGYLDKENEVIYGYQTDVPLKRAIKPFGGVNMVKNALKAVNVPMDKEVEHIFTDYRKTHNTAVFDIYSKEMRAGRSNAIMTGLPDGYGRGRIIGDYRRVALYGTDRLIAQKQKDKVELQKRQMDEPTMKLIGEVADQIKALKQLTQMAKSYGIDITKPAKNAREATQFVYFGYLGSIKEQDGAAMSLGRVDAFLDCFFENDLKNGVLDEAHAQEIIDNLILKLRFARHLRTPEYNDLFAGDPTWVTMSLGGTGSDGRTLVTKTSFRVLNTLYNLGPAPEPNITVLWNKNLPKNFKDFATKVSIDTSSIQYESDALMSARFGDDYGIACCVSAMRIGKDMQFFGARCNLAKLMLYVLNHGKDERTGKQVGPDFGPVPDGPIPFDWMWETYDKAMDWIAKLYVNTMNVIHFCHDQYCYESLQMALHDTDVRRLMAFGVAGLSVVADSFSAIKYAKVTPIRDPKTGLTTDFKVEGEFPKFGNDDDRVDFFARTVTDKLITKLRKTPTYRGATHTLSILTITSNVVYGKKTGSTPDGRKAGQPFAPGCNPMHGREFSGAVASLSSVAKVNYDSCMDGISNTFSIVPNTIGKTLQERQGNLSGLLDGYFSKGAHHLNVNVLKRETLEDAMAHPENYPNLTIRVSGYAVNFVKLTPAQQKEVIARTFHEKM

SEQ ID NO: 18 - DNA, 801 bp, Artificial Sequence
  Synthesized: PFLA 41400040
atgccagctatcgttgatccaactactatggattatatggaagtcaagggcaatgtccattcaactgaaagtttggcttgtcttgaaggtccaggaaacagattccttttatttttaaatggttgtgctgctcgttgcttatactgtagtaatccagatacttgggatgaaactgttggtactccaatgaccgttggccaacttattaagaagattggaaatcttaaaaactactatatcaattctgttggtggtggtggtgtcactgtttctggtggtgaaccattaactcaatttggtttcttatcttgtttcttatatgctgtcaagaagcacttaaatcttcatacctgtgttgaaaccactggtcaaggttgtactaaggcttggaattcagttttacctcatactgacttatgcttagtatgtattaaacatgctattccagaaaaatacgaacaaattactcgtactaagaaattagatagatgtcttaagttccttaaggaattagaaaagagaaacattccatggtggtgtcgttacgttgttcttccaggttacactgattctaaggaagatattgaagctttaattgaattagttaagaacagtccaacttgtgaaagaattgaattccttccataccccgaattaggtaaaaacaaatgggaagaattaggtattgaatatccattaaagaatattaaacaacttaagaaaagtgaaattaaatggatctgtgatatggtccgtgaagctttcaaggaccgtaatattccagttactggtgatacttaa

SEQ ID NO: 19 - protein, 266 aa, Artificial Sequence
  Synthesized: Formate-C acetyltransferase (activating enzyme) (protein) Q6RFH6
MPAIVDPTTMDYMEVKGNVHSTESLACLEGPGNRFLLFLNGCAARCLYCSNPDTWDETVGTPMTVGQLIKKIGNLKNYYINSVGGGGVTVSGGEPLTQFGFLSCFLYAVKKHLNLHTCVETTGQGCTKAWNSVLPHTDLCLVCIKHAIPEKYEQITRTKKLDRCLKFLKELEKRNIPWWCRYVVLPGYTDSKEDIEALIELVKNSPTCERIEFLPYPELGKNKWEELGIEYPLKNIKQLKKSEIKWICDMVREAFKDRNIPVTGDT

SEQ ID NO: 20 - DNA, 1263 bp, Artificial Sequence
  Synthesized: pda1 298058
atgcttgctgcttcattcaaacgccaaccatcacaattggtccgcgggttaggagctgttcttcgcactcccaccaggataggtcatgttcgtaccatggcaactttaaaaacaactgataagaaggcccctgaggacatcgagggctcggacacagtgcaaattgagttgcctgaatcttccttcgagtcgtatatgctagagcctccagacttgtcttatgagacttcgaaagccaccttgttacagatgtataaagatatggtcatcatcagaagaatggagatggcttgtgacgccttgtacaaggccaagaaaatcagaggtttttgccatctatctgttggtcaggaggccattgctgtcggtatcgagaatgccatcacaaaattggattccatcatcacatcttacagatgtcacggtttcacttttatgagaggtgcctcagtgaaagccgttctggctgaattgatgggtagaagagccggtgtctcttatggtaagggtggttccatgcacctttacgctccaggcttctatggtggtaatggtatcgtgggtgcccaggttcctttaggtgcaggtttagcttttgctcaccaatacaagaacgaggacgcctgctctttcactttgtatggtgatggtgcctctaatcaaggtcaagtttttgaatctttcaacatggccaaattatggaatttgcccgtcgtgttttgctgtgagaacaacaagtacggtatgggtaccgccgcttcaagatcctccgcgatgactgaatatttcaagcgtggtcaatatattccaggtttaaaagttaacggtatggatattctagctgtctaccaagcatccaagtttgctaaggactggtgtctatccggcaaaggtcctctcgttctagaatatgaaacctataggtacggtggccattctatgtctgatcccggtactacctacagaactagagacgagattcagcatatgagatccaagaacgatccaattgctggtcttaagatgcatttgattgatctaggtattgccactgaagctgaagtcaaagcttacgacaagtccgctagaaaatacgttgacgaacaagttgaattagctgatgctgctcctcctccagaagccaaattatccatcttgtttgaagacgtctacgtgaaaggtacagaaactccaaccctaagaggtaggatccctgaagatacttgggacttcaaaaagcaaggttttgcctctagggattaa

SEQ ID NO: 21 - protein, 420 aa, Artificial Sequence
  Synthesized: pda1 Protein P16387
MLAASFKRQPSQLVRGLGAVLRTPTRIGHVRTMATLKTTDKKAPEDIEGSDTVQIELPESSFESYMLEPPDLSYETSKATLLQMYKDMVIIRRMEMACDALYKAKKIRGFCHLSVGQEAIAVGIENAITKLDSIITSYRCHGFTFMRGASVKAVLAELMGRRAGVSYGKGGSMHLYAPGFYGGNGIVGAQVPLGAGLAFAHQYKNEDACSFTLYGDGASNQGQVFESFNMAKLWNLPVVFCCENNKYGMGTAASRSSAMTEYFKRGQYIPGLKVNGMDILAVYQASKFAKDWCLSGKGPLVLEYETYRYGGHSMSDPGTTYRTRDEIQHMRSKNDPIAGLKMHLIDLGIATEAEVKAYDKSARKYVDEQVELADAAPPPEAKLSILFEDVYVKGTETPTLRGRIPEDTWDFKKQGFASRD

SEQ ID NO: 22 - DNA, 1101 bp, Artificial Sequence
  Synthesized: pdb1 171428
atgttttccagactgccaacatcattggccagaaatgttgcacgtcgtgccccaacttcttttgtaagaccctctgcagcagcagcagcattgagattctcatcaacaaagacgatgaccgtcagagaggccttgaatagtgccatggcggaagaattggaccgtgatgatgatgtcttccttattggtgaagaagttgcacaatataacggggcttataaggtgtcaaagggtttattggacaggttcggtgaacgtcgtgtggttgacacacctattaccgaatacgggttcacaggtttggccgttggtgccgctttgaagggtttgaagccaattgtagagtttatgtcgttcaatttctctatgcaagctatcgatcatgttgtcaattccgctgcaaagactcactacatgtctggtggtactcaaaaatgtcaaatggtcttcagaggtcctaatggtgctgcagtgggtcttggtgctcaacattcacaggacttttctccttggtacggttccattccagggttaaaggtccttgtcccttattctgctgaagatgctaggggtttgttaaaggccgccatcagagatccaaaccctgttgtatttttagagaacgaattgttgtacggtgaatcttttgaaatctcagaagaagctttatcccctgagttcaccttgccatacaaggctaagatcgaaagagaaggtaccgatatttccattgttacgtacacaagaaacgttcagttttctttggaagccgctgaaattctacaaaagaaatatggtgtctctgcagaagttatcaacttgcgttctattagacctttagatactgaagctatcatcaaaactgtcaagaagacaaaccacttgattactgttgaatccactttcccatcatttggtgttggtgctgaaattgtcgcccaagttatggagtctgaagcctttgattacttggatgctccaatccaaagagttactggtgccgatgttccaacaccttacgctaaagaattagaagatttcgctttccctgatactccaaccatcgttaaagctgtcaaagaagtcttgtcaattgaataa

SEQ ID NO: 23 - protein, 366 aa, Artificial Sequence
  Synthesized: pdb1 Protein P32473
MFSRLPTSLARNVARRAPTSFVRPSAAAAALRFSSTKTMTVREALNSAMAEELDRDDDVFLIGEEVAQYNGAYKVSKGLLDRFGERRVVDTPITEYGFTGLAVGAALKGLKPIVEFMSFNFSMQAIDHVVNSAAKTHYMSGGTQKCQMVFRGPNGAAVGVGAQHSQDFSPWYGSIPGLKVLVPYSAEDARGLLKAAIRDPNPVVFLENELLYGESFEISEEALSPEFTLPYKAKIEREGTDISIVTYTRNVQFSLEAAEILQKKYGVSAEVINLRSIRPLDTEAIIKTVKKTNHLITVESTFPSFGVGAEIVAQVMESEAFDYLDAPIQRVTGADVPTPYAKELEDFAFPDTPTIVKAVKEVLSIE

SEQ ID NO: 24 - DNA, 1449 bp, Artificial Sequence
  Synthesized: pdb1 170971
atgtctgcctttgtcagggtggttccaagaatatccagaagttcagtactcaccagatcattgagactgcaattgagatgctacgcatcgtacccagagcacaccattattggtatgccggcactgtctcctacgatgacgcaaggtaatcttgctgcttggactaagaaggaaggtgaccaattgtctcccggtgaagttattgccgaaatagaaacagacaaggctcaaatggactttgagttccaagaagatggttacttagccaagattctagttcctgaaggtacaaaggacattcctgtcaacaagcctattgccgtctatgtggaggacaaagctgatgtgccagcttttaaggactttaagctggaggattcaggttctgattcaaagaccagtacgaaggctcagcctgccgaaccacaggcagaaaagaaacaagaagcgccagctgaagagaccaagacttctgcacctgaagctaagaaatctgacgttgctgctcctcaaggtaggatttttgcctctccacttgccaagactatcgccttggaaaagggtatttctttgaaggatgttcacggcactggaccccgcggtagaattaccaaggctgacattgagtcatatctagaaaagtcgtctaagcagtcttctcaaaccagtggtgctgccgccgccactcctgccgccgctacctcaagcactactgctggctctgctccatcgccttcttctacagcatcatatgaggatgttccaatttcaaccatgagaagcatcattggagaacgtttattgcaatctactcaaggcattccatcatacatcgtttcctccaagatatccatctccaaacttttgaaattgagacagtccttgaacgctacagcaaacgacaagtacaaactgtccattaatgacctattagtaaaagccatcactgttgcggctaagagggtgccagatgccaatgcctactggttacctaatgagaacgttatccgtaaattcaagaatgtcgatgtctcagtcgctgttgccacaccaacaggattattgacaccaattgtcaagaattgtgaggccaagggcttgtcgcaaatctctaacgaaatcaaggaactagtcaagcgtgccagaataaacaaattggcaccagaggaattccaaggtgggaccatttgcatatccaatatgggcatgaataatgctgttaacatgtttacttcgattatcaacccaccacagtctacaatcttggccatcgctactgttgaaagggtcgctgtggaagacgccgctgctgagaacggattctcctttgataaccaggttaccataacagggacctttgatcatagaaccattgatggcgccaaaggtgcagaattcatgaaggaattgaaaactgttattgaaaatcctttggaaatgctattgtga

SEQ ID NO: 25 - protein, 482 aa, Artificial Sequence
  Synthesized: lat1 Protein P12695
MSAFVRVVPRISRSSVLTRSLRLQLRCYASYPEHTIIGMPALSPTMTQGNLAAWTKKEGDQLSPGEVIAEIETDKAQMDFEFQEDGYLAKILVPEGTKDIPVNKPIAVYVEDKADVPAFKDFKLEDSGSDSKTSTKAQPAEPQAEKKQEAPAEETKTSAPEAKKSDVAAPQGRIFASPLAKTIALEKGISLKDVHGTGPRGRITKADIESYLEKSSKQSSQTSGAAAATPAAATSSTTAGSAPSPSSTASYEDVPISTMRSIIGERLLQSTQGIPSYIVSSKISISKLLKLRQSLNATANDKYKLSINDLLVKAITVAAKRVPDANAYWLPNENVIRKFKNVDVSVAVATPTGLLTPIVKNCEAKGLSQISNEIKELVKRARINKLAPEEFQGGTICISNMGMNNAVNMFTSIINPPQSTILAIATVERVAVEDAAAENGFSFDNQVTITGTFDHRTIDGAKGAEFMKELKTVIENPLEMLL

SEQ ID NO: 26 - DNA, 1500 bp, Artificial Sequence
  Synthesized: lpd1 171847
atgttaagaatcagatcactcctaaataataagcgtgccttttcgtccacagtcaggacattgaccattaacaagtcacatgatgtagtcatcatcggtggtggccctgctggttacgtggctgctatcaaagctgctcaattgggatttaacactgcatgtgtagaaaaaagaggcaaattaggcggtacctgtcttaacgttggatgtatcccctccaaagcacttctaaataattctcatttattccaccaaatgcatacggaagcgcaaaagagaggtattgacgtcaacggtgatatcaaaattaacgtagcaaacttccaaaaggctaaggatgacgctgttaagcaattaactggaggtattgagcttctgttcaagaaaaataaggtcacctattataaaggtaatggttcattcgaagacgaaacgaagatcagagtaactcccgttgatgggttggaaggcactgtcaaggaagaccacatactagatgttaagaacatcatagtcgccacgggctctgaagttacacccttccccggtattgaaatagatgaggaaaaaattgtctcttcaacaggtgctctttcgttaaaggaaattcccaaaagattaaccatcattggtggaggaatcatcggattggaaatgggttcagtttactctagattaggctccaaggttactgtagtagaatttcaacctcaaattggtgcatctatggacggcgaggttgccaaagccacccaaaagttcttgaaaaagcaaggtttggacttcaaattaagcaccaaagttatttctgcaaagagaaacgacgacaagaacgtcgtcgaaattgttgtagaagatactaaaacgaataagcaagaaaatttggaagctgaagttttgctggttgctgttggtagaagaccttacattgctggcttaggggctgaaaagattggattagaagtagacaaaaggggacgcctagtcattgatgaccaatttaattccaagttcccacacattaaagtggtaggagatgttacatttggtccaatgctggctcacaaagccgaagaggaaggtattgcagctgtcgaaatgttgaaaactggtcacggtcatgtcaactataacaacattccttcggtcatgtattctcacccagaagtagcatgggttggtaaaaccgaagagcaattgaaagaagccggcattgactataaaattggtaagttcccctttgcggccaattcaagagccaagaccaaccaagacactgaaggtttcgtgaagattttgatcgattccaagaccgagcgtattttgggggctcacattatcggtccaaatgccggtgaaatgattgctgaagctggcttagccttagaatatggcgcttccgcagaagatgttgctagggtctgccatgctcatcctactttgtccgaagcatttaaggaagctaacatggctgcctatgataaagctattcattgttga

SEQ ID NO: 27 - protein, 499 aa, Artificial Sequence
  Synthesized: pdx1 Protein P09624
MLRIRSLLNNKRAFSSTVRTLTINKSHDVVIIGGGPAGYVAAIKAAQLGFNTACVEKRGKLGGTCLNVGCIPSKALLNNSHLFHQMHTEAQKRGIDVNGDIKINVANFQKAKDDAVKQLTGGIELLFKKNKVTYYKGNGSFEDETKIRVTPVDGLEGTVKEDHILDVKNIIVATGSEVTPFPGIEIDEEKIVSSTGALSLKEIPKRLTIIGGGIIGLEMGSVYSRLGSKVTVVEFQPQIGASMDGEVAKATQKFLKKQGLDFKLSTKVISAKRNDDKNVVEIVVEDTKTNKQENLEAEVLLVAVGRRPYIAGLGAEKIGLEVDKRGRLVIDDQFNSKFPHIKVVGDVTFGPMLAHKAEEEGIAAVEMLKTGHGHVNYNNIPSVMYSHPEVAWVGKTEEQLKEAGIDYKIGKFPFAANSRAKTNQDTEGFVKILIDSKTERILGAHIIGPNAGEMIAEAGLALEYGASAEDVARVCHAHPTLSEAFKEANMAAYDKAIHC

SEQ ID NO: 28 - DNA, 1233 bp, Artificial Sequence
  Synthesized: pdx1 172267
atgctaagtgcaatttccaaagtctccactttaaaatcatgtacaagatatttaaccaaatgcaactatcatgcatcagctaaattacttgctgtaaagacattttcaatgcctgcaatgtctcctactatggagaaaggggggattgtgtcttggaaatataaagttggcgaaccattcagcgcgggcgatgtgatattagaagtggaaacagataaatctcaaattgatgtggaagcactggacgatggtaaactagctaagatcctgaaagatgaaggctctaaagatgttgatgttggtgaacctattgcttatattgctgatgttgatgatgatttagctactataaagttaccccaagaggccaacaccgcaaatgcgaaatctattgaaattaagaagccatccgcagatagtactgaagcaacacaacaacatttaaaaaaagccacagttacaccaataaaaaccgttgacggcagccaagccaatcttgaacagacgctattaccatccgtgtcattactactggctgagaacaatatatccaaacaaaaggctttgaaggaaattgcgccatctggttccaacggtagactattaaagggtgatgtgctagcatacctagggaaaataccacaagattcggttaacaaggtaacagaatttatcaagaagaacgaacgtctcgatttatcgaacattaaacctatacagctcaaaccaaaaatagccgagcaagctcaaacaaaagctgccgacaagccaaagattactcctgtagaatttgaagagcaattagtgttccatgctcccgcctctattccgtttgacaaactgagtgaatcattgaactctttcatgaaagaagcttaccagttctcacacggaacaccactaatggacacaaattcgaaatactttgaccctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttga

SEQ ID NO: 29 - protein, 410 aa, Artificial Sequence
  Synthesized: pdx1 Protein P16451
MLSAISKVSTLKSCTRYLTKCNYHASAKLLAVKTFSMPAMSPTMEKGGIVSWKYKVGEPFSAGDVILEVETDKSQIDVEALDDGKLAKILKDEGSKDVDVGEPIAYIADVDDDLATIKLPQEANTANAKSIEIKKPSADSTEATQQHLKKATVTPIKTVDGSQANLEQTLLPSVSLLLAENNISKQKALKEIAPSGSNGRLLKGDVLAYLGKIPQDSVNKVTEFIKKNERLDLSNIKPIQLKPKIAEQAQTKAADKPKITPVEFEEQLVFHAPASIPFDKLSESLNSFMKEAYQFSHGTPLMDTNSKYFDPIFEDLVTLSPREPRFKFSYDLMQIPKANNMQDTYGQEDIFDLLTGSDATASSVRPVEKNLPEKNEYILALNVSVNNKKFNDAEAKAKRFLDYVRELESF

SEQ ID NO: 30 - DNA, 1116 bp, Artificial Sequence
  Synthesized: pdhA 327533853
atggcaaaggctaagaaacaaaaacctattgactttaaagagctaatggctaaagtcgacgctgatttcccaactttccaaatcttggatcaagatggaaaaattgtgaatgaagatttagtacctgatttatcggatgaggaattagttgaattaatgacacgcatggtttggtctcgtgtgttagaccaacgttctactgcattaaaccgtcaaggacgcttaggattcttcgcgccaacagctggacaagaagcaagccaattggcaagtcaatttgcaatggaaaaagaagactacttactaccaggttaccgtgatgtacctcaattagtacaacatggtttaccattaagagaagctttcttatggtctcgtggtcacgtagcagggaactactacgcggaagatttaaatgcattaccaccacaaattatcattggtgctcaatacatccaagcagctggtgttgctttaggattgaaaaaacgtggaaaagaaaatgttgtcttcacttatactggtgacggcggttcttcacaaggggacttctatgaagcaattaactttgctggtgcttaccaagcaaacggtgtcttcattatccaaaacaatggttttgcgatttctacacctcgtgaaaaacaaacagcggctaaaactttagctcaaaaagctgttgcagcaggaattcctggtattcaagttgatggtatggatccattagcagtttacgcaattgcaaaagaagcacgtgattggtcagctgcaggaaacggtccagttttaattgaaacattaacctatcgttatggtccacatactttatctggagacgatccaacacgttaccgttcaaaagaaatggatgacgaatgggtacaaaaagatccattgactcgtttccgtaaatatctaacagataaaggcttatggtctgaagcaaaagaagaagaaattattgaaaaaacaaaagaagaaatcaaagtagcgattgcagaagcggataaagcgccaaaacaaaaagtttctgatttcttgaaaaatatgtttgaagttcaacctcaaacaattaaagaacaaattgcattttatgaagcgaaggagtcgaaataa

SEQ ID NO: 31 - protein, 371 aa, Artificial Sequence
  Synthesized: pdhA Protein F2MRX7
MAKAKKQKPIDFKELMAKVDADFPTFQILDQDGKIVNEDLVPDLSDEELVELMTRMVWSRVLDQRSTALNRQGRLGFFAPTAGQEASQLASQFAMEKEDYLLPGYRDVPQLVQHGLPLREAFLWSRGHVAGNYYAEDLNALPPQIIIGAQYIQAAGVALGLKKRGKENVVFTYTGDGGSSQGDFYEAINFAGAYQANGVFIIQNNGFAISTPREKQTAAKTLAQKAVAAGIPGIQVDGMDPLAVYAIAKEARDWSAAGNGPVLIETLTYRYGPHTLSGDDPTRYRSKEMDDEWVQKDPLTRFRKYLTDKGLWSEAKEEEIIEKTKEEIKVAIAEADKAPKQKVSDFLKNMFEVQPQTIKEQIAFYEAKESK

SEQ ID NO: 32 - DNA, 978 bp, Artificial Sequence
  Synthesized: pdhB 327533853
atggcacaaaaaactatgatccaagcaattacagatgccttagctcttgaattagagaaagacgaaaatgtcttaatcttcggtgaagacgttggtaacaacggtggggttttccgtgcaactgaaggtttacaagaaaaatttggtgaagaccgcgtcttcgatacacctttagctgaatctggtatcggtggattggctttcggtcttgccttgcaaggttaccgtccagttcctgaaatccaattctttggtttcgtttttgaagtatttgacgaaatcgttggtcaaatggctcgtacgcgttaccgtatgggtggaactcgtaatatgccaattactgttcgtgccccatttggtggtggtgttcatacaccagaacttcactcagataacttagaaggattaatcgcacaatcaccaggtgttcgtgttgttattccatcaaacccttacgatgcaaaaggactattaatttcatctattcgtagcaacgatccagttgtttacttagagcacatgaaattataccgttcattccgtgaggaagtgccagacgaagcttatgaagtgcctttagataaagcggctgtaactcgtgaaggaacagacgtatcaatcatcacttacggtgctatggttcgtgaagcgattaaagcagctgatagcttagcgaaagacaatatttcagcagaaatcattgacttacgtacagtggctcctttagatgtggaaacaattattaactctgttgaaaaaactggccgtgtggttgtcgttcaagaagcacaaaaacaagctggcgttggcgctatggttgtttctgaaatttctgaacgtgccgtattatcattagaagcaccaatcggacgtgtatctgctccagatacaatcttcccattcggacaagcagaaaatatctggttaccaaatgcgaaagatatcgaagcaaaagctagagaaatcgtcgaattttaa

SEQ ID NO: 33 - protein, 325 aa, Artificial Sequence
  Synthesized: pdhB Protein F2MRX8
MAQKTMIQAITDALALELEKDENVLIFGEDVGNNGGVFRATEGLQEKFGEDRVFDTPLAESGIGGLAFGLALQGYRPVPEIQFFGFVFEVFDEIVGQMARTRYRMGGTRNMPITVRAPFGGGVHTPELHSDNLEGLIAQSPGVRVVIPSNPYDAKGLLISSIRSNDPVVYLEHMKLYRSFREEVPDEAYEVPLDKAAVTREGTDVSIITYGAMVREAIKAADSLAKDNISAEIIDLRTVAPLDVETIINSVEKTGRVVVVQEAQKQAGVGAMVVSEISERAVLSLEAPIGRVSAPDTIFPFGQAENIWLPNAKDIEAKAREIVEF

SEQ ID NO: 34 - DNA, 1620 bp, Artificial Sequence
  Synthesized: aceF 327533853
atggcttatcagtttaaattaccggatatcggtgaagggattgccgaaggcgaaatcgttaaatggtttgtaaaacctggcgatacaatcaacgaagacgatacgttattagaagtacaaaatgacaaatcagtggaagaaattccatcaccagtaacaggtactgtaaaaaatatcgttgtaccagaaggaacagttgcaaacgttggtgacgtgttaatcgaaatcgacgcacctggtcacgaagataacgatgcagcaccagcagctcctgcacaagaacaaacaccagcacaacctgctgctgtaccaacaaccgaagcagctggcggatttttccaattcaaattaccagacatcggtgaaggaattgccgaaggcgaaatcgttaaatggttcgttaaagcgggcgacacaattaatgaagatgattcattattagaagtacaaaatgacaaatcagtagaagaaattccatcaccagtaacaggtactgtaaaaaatatcgttgtaccagaaggaacagttgccaatgtgggtgacgtgttagttgaaattgacgcacctggtcataattcagcagcaccggcagccgcagcaccagctactgacgctcctaaagcggaagcatcagctccagccgcttcaacaggcgtagttgcagccgctgatccaaacaaacgcgttttagcaatgccatctgttcgtcagtatgcgcgtgaaaaagacgttgatattacacaagtaactgcaactggtaaaggtggccgtgtcattaaagcggatattgatgcctttgtttctggtggctctcaagcagcaccagctactgaagctgccgcaacagaagcagcacctaaagcggaagcagctgcacctaaagcagcgccaaaagcctttacttctgatttaggcgaaatggaaacacgtgaaaaaatgacaccaacacgtaaagcaattgctaaagcaatggttaacagcaaacacactgctcctcacgtaacattacatgatgaagtagaagtttctaaattatgggatcaccgtaagaaatttaaagatgttgctgctgcaaatggtacaaaattaacattcttaccatacgttgtaaaagcattgacttcaactgttcaaaaattcccaatcttgaatgcatcaattgatgacgcagcacaagaaattgtttacaaaaattactttaacattggtatcgctactgatacagatcatggcttatatgtaccaaatgttaaaaatgctaatacgaagagcatgtttgctatcgctgatgaaatcaacgaaaaagcagcattggctatcgaaggcaaattaactgcacaagatatgcgtgatggtacaatcacaattagtaacattggttcagtcggtggcggctggtttacaccagtaatcaactaccctgaagttgctattttaggcgttggtacaattgcacaagaaccagttgttaatgcagacggcgaaatcgttgtgggacgcatgatgaaattatcattaagctttgaccaccgtatcgttgacggcgcaactgctcaaaaagcaatgaacaacattaaacgcttattagctgatccagaattactattaatggaaggatga

SEQ ID NO: 35 - protein, 539 aa, Artificial Sequence
  Synthesized: aceF Protein F2MRX9
MAYQFKLPDIGEGIAEGEIVKWFVKPGDTINEDDTLLEVQNDKSVEEIPSPVTGTVKNIVVPEGTVANVGDVLIEIDAPGHEDNDAAPAAPAQEQTPAQPAAVPTTEAAGGFFQFKLPDIGEGIAEGEIVKWFVKAGDTINEDDSLLEVQNDKSVEEIPSPVTGTVKNIVVPEGTVANVGDVLVEIDAPGHNSAAPSVAAPATDAPKAEASAPAASTGVVAAADPNKRVLAMPSVRQYAREKDVDITQVTATGKGGRVIKADIDAFVSGGSQAAPATEAAATEAAPKAEAAAPKAAPKAFTSDLGEMETREKMTPTRKAIAKAMVNSKHTAPHVTLHDEVEVSKLWDHRKKFKDVAAANGTKLTFLPYVVKALTSTVQKFPILNASIDDAAQEIVYKNYFNIGIATDTDHGLYVPNVKNANTKSMFAIADEINEKAALAIEGKLTAQDMRDGTITISNIGSVGGGWFTPVINYPEVAILGVGTIAQEPVVNADGEIVVGRMMKLSLSFDHRIVDGATAQKAMNNIKRLLADPELLLMEG

SEQ ID NO: 36 - DNA, 1407 bp, Artificial Sequence
  Synthesized: Ipd 327533853
atggtagtaggagatttcgccattgaactagatacagtcgtaatcggagctggtcctggaggatacgttgccgcaattcgtgccgcagaaatgggtcaaaaagttgcgattatcgaacgtgaatacatcggaggcgtttgtttaaacgttggatgtattccttcaaaagctttaattgctgctggacatcattaccaagaagcacaagattcttcaacttttggtgtaacagctaaaggagtcgaattagactttgcaaaaacacaagactggaaagataacacagttgtaaaatcattaacaggcggcgttggcatgttattgaaaaaacacaaagtagaaattattgaaggcgaagcattcttcgttgacgaaaatacattgcgtgttattcacccagactcagcacaaacttactcattcaataatgctattgtagcaacaggttctcgtccaattgaaatcccaggattcaaatttggcggacgcgtgttagattctacaggcggtttaaacttaaaagaagttcctaaaaaattcgttattatcggtggcggtgtcatcggtgctgaattaggtggcgcttatgctaacttaggttcagaagtaacaattttagaaggtagcccatcaattttaccaacttatgaaaaagatatggttaaagttgtcacagacgacttcaagaagaaaaacgtaacaatcgtgacttctgcaatggctaaagaagctgttgacaatggcgatagcgtcactgttaaatatgaagttaacggaaaagaagaaagtgttgaagcagattacgtaatggtcactgttggacgtcgtccaaacacagacgacttaggcttagaacaagcgggcgttgaaattggcgaacgtggtttaatcccagttgacaaccaaggacgtactaacgtgaaaaacatcttcgcaatcggcgacatcgtaccaggtgctgcgttagcgcataaagcaagctacgaagcaaaaattgctgctgaagcaatttctggtaagaaagttgcagttgattacaaagcaatgccagctgttgcctttactgatccagaattggcaagcgttggtatgactgttgcagaagcaaaagaagcgggaatcgaagcaaaaggctacaaattcccatttgctggtaacggccgtgcaatctctttagataaaactgaaggattcatgcgtttagttacaactgtagaagacaatgtcatcatcggtgcacaaattgccggtgtcggtgcaagtgacatgatttctgaattagctttagctattgaatctggcatgaatgcagaagacattgctttaacaatccacccacacccatcattgggcgaaattactatggatacagctgaattggctttaggtttaccaattcatatttaa

SEQ ID NO: 37 - protein, 468 aa, Artificial Sequence
  Synthesized: Ipd Protein F2MRY0
MVVGDFAIELDTVVIGAGPGGYVAAIRAAEMGQKVAIIEREYIGGVCLNVGCIPSKALIAAGHHYQEAQDSSTFGVTAKGVELDFAKTQDWKDNTVVKSLTGGVGMLLKKHKVEIIEGEAFFVDENTLRVIHPDSAQTYSFNNAIVATGSRPIEIPGFKFGGRVLDSTGGLNLKEVPKKFVIIGGGVIGAELGGAYANLGSEVTILEGSPSILPTYEKDMVKVVTDDFKKKNVTIVTSAMAKEAVDNGDSVTVKYEVNGKEESVEADYVMVTVGRRPNTDDLGLEQAGVEIGERGLIPVDNQGRTNVKNIFAIGDIVPGAALAHKASYEAKIAAEAISGKKVAVDYKAMPAVAFTDPELASVGMTVAEAKEAGIEAKGYKFPFAGNGRAISLDKTEGFMRLVTTVEDNVIIGAQIAGVGASDMISELALAIESGMNAEDIALTIHPHPSLGEITMDTAELALGLPIHI

SEQ ID NO: 38 - DNA, 1179 bp, Artificial Sequence
  Synthesized: thIA 3309200
atgaaagaagttgtaatagctagtgcagtaagaacagcgattggatcttatggaaagtctcttaaggatgtaccagcagtagatttaggagctacagctataaaggaagcagttaaaaaagcaggaataaaaccagaggatgttaatgaagtcattttaggaaatgttcttcaagcaggtttaggacagaatccagcaagacaggcatcttttaaagcaggattaccagttgaaattccagctatgactattaataaggtttgtggttcaggacttagaacagttagcttagcagcacaaattataaaagcaggagatgctgacgtaataatagcaggtggtatggaaaatatgtctagagctccttacttagcgaataacgctagatggggatatagaatgggaaacgctaaatttgttgatgaaatgatcactgacggattgtgggatgcatttaatgattaccacatgggaataacagcagaaaacatagctgagagatggaacatttcaagagaagaacaagatgagtttgctcttgcatcacaaaaaaaagctgaagaagctataaaatcaggtcaatttaaagatgaaatagttcctgtagtaattaaaggcagaaagggagaaactgtagttgatacagatgagcaccctagatttggatcaactatagaaggacttgcaaaattaaaacctgccttcaaaaaagatggaacagttacagctggtaatgcatcaggattaaatgactgtgcagcagtacttgtaatcatgagtgcagaaaaagctaaagagcttggagtaaaaccacttgctaagatagtttcttatggttcagcaggagttgacccagcaataatgggatatggacctttctatgcaacaaaagcagctattgaaaaagcaggttggacagttgatgaattagatttaatagaatcaaatgaagcttttgcagctcaaagtttagcagtagcaaaagatttaaaatttgatatgaataaagtaaatgtaaatggaggagctattgcccttggtcatccaattggagcatcaggtgcaagaatactcgttactcttgtacacgcaatgcaaaaaagagatgcaaaaaaaggcttagcaactttatgtataggtggcggacaaggaacagcaatattgctagaaaagtgctag

SEQ ID NO: 39 - protein, 392 aa, Artificial Sequence
  Synthesized: thIA Protein P45359
MKEVVIASAVRTAIGSYGKSLKDVPAVDLGATAIKEAVKKAGIKPEDVNEVILGNVLQAGLGQNPARQASFKAGLPVEIPAMTINKVCGSGLRTVSLAAQIIKAGDADVIIAGGMENMSRAPYLANNARWGYRMGNAKFVDEMITDGLWDAFNDYHMGITAENIAERWNISREEQDEFALASQKKAEEAIKSGQFKDEIVPVVIKGRKGETVVDTDEHPRFGSTIEGLAKLKPAFKKDGTVTAGNASGLNDCAAVLVIMSAEKAKELGVKPLAKIVSYGSAGVDPAIMGYGPFYATKAAIEKAGWTVDELDLIESNEAFAAQSLAVAKDLKFDMNKVNVNGGAIALGHPIGASGARILVTLVHAMQKRDAKKGLATLCIGGGQGTAILLEKC

SEQ ID NO: 40 - DNA, 1179 bp, Artificial Sequence; identical to SEQ ID NO: 38
  Synthesized: Erg10 3309200

SEQ ID NO: 41 - protein, 392 aa, Artificial Sequence; identical to SEQ ID NO: 39
  Synthesized: Erg10 Protein P45359

SEQ ID NO: 42 - DNA, 651 bp, Artificial Sequence
  Synthesized: atoA 48994873
atggatgcgaaacaacgtattgcgcgccgtgtggcgcaagagcttcgtgatggtgacatcgttaacttagggatcggtttacccacaatggtcgccaattatttaccggagggtattcatatcactctgcaatcggaaaacggcttcctcggtttaggcccggtcacgacagcgcatccagatctggtgaacgctggcgggcaaccgtgcggtgttttacccggtgcagccatgtttgatagcgccatgtcatttgcgctaatccgtggcggtcatattgatgcctgcgtgctcggcggtttgcaagtagacgaagaagcaaacctcgcgaactgggtagtgcctgggaaaatggtgcccggtatgggtggcgcgatggatctggtgaccgggtcgcgcaaagtgatcatcgccatggaacattgcgccaaagatggttcagcaaaaattttgcgccgctgcaccatgccactcactgcgcaacatgcggtgcatatgctggttactgaactggctgtctttcgttttattgacggcaaaatgtggctcaccgaaattgccgacgggtgtgatttagccaccgtgcgtgccaaaacagaagctcggtttgaagtcgccgccgatctgaatacgcaacggggtgatttatga

SEQ ID NO: 43 - protein, 216 aa, Artificial Sequence
  Synthesized: atoA Protein P76459
MDAKQRIARRVAQELRDGDIVNLGIGLPTMVANYLPEGIHITLQSENGFLGLGPVTTAHPDLVNAGGQPCGVLPGAAMFDSAMSFALIRGGHIDACVLGGLQVDEEANLANWVVPGKMVPGMGGAMDLVTGSRKVIIAMEHCAKDGSAKILRRCTMPLTAQHAVHMLVTELAVFRFIDGKMWLTEIADGCDLATVRAKTEARFEVAADLNTQRGDL

SEQ ID NO: 44 - DNA, 663 bp, Artificial Sequence
  Synthesized: atoD 48994873
atgaaaacaaaattgatgacattacaagacgccaccggcttctttcgtgacggcatgaccatcatggtgggcggatttatggggattggcactccatcccgcctggttgaagcattactggaatctggtgttcgcgacctgacattgatagccaatgataccgcgtttgttgataccggcatcggtccgctcatcgtcaatggtcgagtccgcaaagtgattgcttcacatatcggcaccaacccggaaacaggtcggcgcatgatatctggtgagatggacgtcgttctggtgccgcaaggtacgctaatcgagcaaattcgctgtggtggagctggacttggtggttttctcaccccaacgggtgtcggcaccgtcgtagaggaaggcaaacagacactgacactcgacggtaaaacctggctgctcgaacgcccactgcgcgccgacctggcgctaattcgcgctcatcgttgcgacacacttggcaacctgacctatcaacttagcgcccgcaactttaaccccctgatagcccttgcggctgatatcacgctggtagagccagatgaactggtcgaaaccggcgagctgcaacctgaccatattgtcacccctggtgccgttatcgaccacatcatcgtttcacaggagagcaaataa

SEQ ID NO: 45 - protein, 220 aa, Artificial Sequence
  Synthesized: atoD Protein P76458
MKTKLMTLQDATGFFRDGMTIMVGGFMGIGTPSRLVEALLESGVRDLTLIANDTAFVDTGIGPLIVNGRVRKVIASHIGTNPETGRRMISGEMDVVLVPQGTLIEQIRCGGAGLGGFLTPTGVGTVVEEGKQTLTLDGKTWLLERPLRADLALIRAHRCDTLGNLTYQLSARNFNPLIALAADITLVEPDELVETGELQPDHIVTPGAVIDHIIVSQESK

SEQ ID NO: 46 - DNA, 735 bp, Artificial Sequence
  Synthesized: adc 6466901
atgttaaaggatgaagtaattaaacaaattagcacgccattaacttcgcctgcatttcctagaggaccctataaatttcataatcgtgagtattttaacattgtatatcgtacagatatggatgcacttcgtaaagttgtgccagagcctttagaaattgatgagcccttagtcaggtttgaaattatggcaatgcatgatacgagtggacttggttgttatacagaaagcggacaggctattcccgtaagctttaatggagttaagggagattatcttcatatgatgtatttagataatgagcctgcaattgcagtaggaagggaattaagtgcatatcctaaaaagctcgggtatccaaagctttttgtggattcagatactttagtaggaactttagactatggaaaacttagagttgcgacagctacaatggggtacaaacataaagccttagatgctaatgaagcaaaggatcaaatttgtcgccctaattatatgttgaaaataatacccaattatgatggaagccctagaatatgtgagcttataaatgcgaaaatcacagatgttaccgtacatgaagcttggacaggaccaactcgactgcagttatttgatcacgctatggcgccacttaatgatttgccagtaaaagagattgtttctagctctcacattcttgcagatataatattgcctagagctgaagttatatatgattatcttaagtaa

SEQ ID NO: 47 - protein, 244 aa, Artificial Sequence
  Synthesized: adc Protein P23670
MLKDEVIKQISTPLTSPAFPRGPYKFHNREYFNIVYRTDMDALRKVVPEPLEIDEPLVRFEIMAMHDTSGLGCYTESGQAIPVSFNGVKGDYLHMMYLDNEPAIAVGRELSAYPKKLGYPKLFVDSDTLVGTLDYGKLRVATATMGYKHKALDANEAKDQICRPNYMLKIIPNYDGSPRICELINAKITDVTVHEAWTGPTRLQLFDHAMAPLNDLPVKEIVSSSHILADIILPRAEVIYDYLK

SEQ ID NO: 48 - DNA, 741 bp, Artificial Sequence
  Synthesized: adc 149901357
atgttagaaagtgaagtatctaaacaaattacaactccacttgctgctccagcgtttcctagaggaccatataggtttcacaatagagaatatctaaacattatttatcgaactgatttagatgctcttcgaaaaatagtaccagagccacttgaattagatagagcatatgttagatttgaaatgatggctatgcctgatacaaccggactaggctcatatacagaatgtggtcaagctattccagtaaaatataatggtgttaagggtgactacttgcatatgatgtatctagataatgaacctgctattgctgttggaagagaaagtagcgcttatccaaaaaagcttggctatccaaagctatttgttgattcagatactttagttgggacacttaaatatggtacattaccagtagctactgcaacaatgggatataagcacgagcctctagatcttaaagaagcctatgctcaaattgcaagacccaattttatgctaaaaatcattcaaggttacgatggtaagccaagaatttgtgaactaatatgtgcagaaaatactgatataactattcacggtgcttggactggaagtgcacgtctacaattatttagccatgcactagctcctcttgctgatttacctgtattagagattgtatcagcatctcatatcctcacagatttaactcttggaacacctaaggttgtacatgattatctttcagtaaaataa

SEQ ID NO: 49 - protein, 246 aa, Artificial Sequence
  Synthesized: adc Protein A6M020
MLESEVSKQITTPLAAPAFPRGPYRFHNREYLNIIYRTDLDALRKIVPEPLELDRAYVRFEMMAMPDTTGLGSYTECGQAIPVKYNGVKGDYLHMMYLDNEPAIAVGRESSAYPKKLGYPKLFVDSDTLVGTLKYGTLPVATATMGYKHEPLDLKEAYAQIARPNFMLKIIQGYDGKPRICELICAENTDITIHGAWTGSARLQLFSHALAPLADLPVLEIVSASHILTDLTLGTPKVVHDYLSVK

SEQ ID NO: 50 - DNA, 1056 bp, Artificial Sequence
  Synthesized: adh 60592972
atgaaaggttttgcaatgctaggtattaataagttaggatggatcgaaaaagaaaggccagttgcgggttcatatgatgctattgtacgcccattagcagtatctccgtgtacatcagatatacatactgtttttgagggagctcttggagataggaagaatatgattttagggcatgaagctgtaggtgaagttgttgaagtaggaagtgaagtgaaggattttaaacctggtgacagagttatagttccttgtacaactccagattggagatctttggaagttcaagctggttttcaacagcactcaaacggtatgctcgcaggatggaaattttcaaatttcaaggatggagtttttggtgaatattttcatgtaaatgatgcggatatgaatcttgcgattctacctaaagacatgccattagaaaatgctgttatgataacagatatgatgactactggatttcatggagcagaacttgcagatattcaaatgggttcaagtgttgtggtaattggcattggagctgttggcttaatgggaatagcaggtgctaaattacgtggagcaggtagaataattggagtggggagcaggccgatttgtgttgaggctgcaaaattttatggagcaacagatattctaaattataaaaatggtcatatagttgatcaagttatgaaattaacgaatggaaaaggcgttgaccgcgtaattatggcaggcggtggttctgaaacattatcccaagcagtatctatggttaaaccaggaggaataatttctaatataaattatcatggaagtggagatgctttactaataccacgtgtagaatggggatgtggaatggctcacaagactataaaaggaggtctttgtcctgggggacgtttgagagcagaaatgttaagagatatggtagtatataatcgtgttgatctaagtaaattagttacacatgtatatcatggatttgatcacatagaagaagcactgttattaatgaaagacaagccaaaagacttaattaaagcagtagttatattataa

SEQ ID NO: 51 - protein, 351 aa, Artificial Sequence
  Synthesized: adh Protein P25984
MKGFAMLGINKLGWIEKERPVAGSYDAIVRPLAVSPCTSDIHTVFEGALGDRKNMILGHEAVGEVVEVGSEVKDFKPGDRVIVPCTTPDWRSLEVQAGFQQHSNGMLAGWKFSNFKDGVFGEYFHVNDADMNLAILPKDMPLENAVMITDMMTTGFHGAELADIQMGSSVVVIGIGAVGLMGIAGAKLRGAGRIIGVGSRPICVEAAKFYGATDILNYKNGHIVDQVMKLTNGKGVDRVIMAGGGSETLSQAVSMVKPGGIISNINYHGSGDALLIPRVEWGCGMAHKTIKGGLCPGGRLRAEMLRDMVVYNRVDLSKLVTHVYHGFDHIEEALLLMKDKPKDLIKAVVIL

SEQ ID NO: 52 - DNA, 414 bp, Artificial Sequence
  Synthesized: mgsA 1146223
atgaaaattgctttgatcgcgcatgacaagaaaaaacaggatatggttcaatttacgactgcctatcgggatattttaaagaatcatgatctatacgcaaccggaaccacagggttgaaaattcatgaggcgacaggtcttcaaattgaacgttttcaatccggccctttagggggagaccagcaaatcggtgcactgatcgctgccaatgcactcgatcttgtcatttttttgcgcgacccgctgaccgcgcagccgcatgaaccggatgtctcggcattaatccgtttatgtgatgtgtattccattccgctcgccacaaatatgggtactgcggaaattcttgtgcgcacacttgatgaaggtgttttcgaattccgtgaccttcttcggggagaagagccgaatgtataa

SEQ ID NO: 53 - protein, 137 aa, Artificial Sequence
  Synthesized: mgsA Protein P42980
MKIALIAHDKKKQDMVQFTTAYRDILKNHDLYATGTTGLKIHEATGLQIERFQSGPLGGDQQIGALIAANALDLVIFLRDPLTAQPHEPDVSALIRLCDVYSIPLATNMGTAEILVRTLDEGVFEFRDLLRGEEPNV

SEQ ID NO: 54 - DNA, 459 bp, Artificial Sequence
  Synthesized: mgsA 1845160
atggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaa

SEQ ID NO: 55 - protein, 152 aa, Artificial Sequence [sequence omitted]

SEQ ID NO: 56 - DNA, 459 bp, Artificial Sequence
  Synthesized: mgsA*
atggaactgacgactcgcactttacctgcgcggaaacatattgcgctggtggcacacgatcaatgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaa

SEQ ID NO: 57 - protein, 152 aa, Artificial Sequence
  Synthesized: msgA* Protein P0A731
MELTTRTLPARKHIALVAHDQCKQMLMSWVERHQPLLEQHVLYATGTTGNLISRATGMNVNAMLSGPMGGDQQVGALISEGKIDVLIFFWDPLNAVPHDPDVKALLRLATVWNIPVATNVATADFIIQSPHFNDAVDILIPDYQRYLADRLK

SEQ ID NO: 58 - DNA, 1104 bp, Artificial Sequence
  Synthesized: gldA 12933659
atggaccgcattattcaatcaccgggtaaatacatccagggcgctgatgtgattaatcgtctgggcgaatacctgaagccgctggcagaacgctggttagtggtgggtgacaaatttgttttaggttttgctcaatccactgtcgagaaaagctttaaagatgctggactggtagtagaaattgcgccgtttggcggtgaatgttcgcaaaatgagatcgaccgtctgcgtggcatcgcggagactgcgcagtgtggcgcaattctcggtatcggtggcggaaaaaccctcgatactgccaaagcactggcacatttcatgggtgttccggtagcgatcgcaccgactatcgcctctaccgatgcaccgtgcagcgcattgtctgttatctacaccgatgagggtgagtttgaccgctatctgctgttgccaaataacccgaatatggtcattgtcgacaccaaaatcgtcgctggcgcacctgcacgtctgttagcggcgggtatcggcgatgcgctggcaacctggtttgaagcgcgtgcctgctctcgtagcggcgcgaccaccatggcgggcggcaagtgcacccaggctgcgctggcactggctgaactgtgctacaacaccctgctggaagaaggcgaaaaagcgatgcttgctgccgaacagcatgtagtgactccggcgctggagcgcgtgattgaagcgaacacctatttgagcggtgttggttttgaaagtggtggtctggctgcggcgcacgcagtgcataacggcctgaccgctatcccggacgcgcatcactattatcacggtgaaaaagtggcattcggtacgctgacgcagctggttctggaaaatgcgccggtggaggaaatcgaaaccgtagctgcccttagccatgcggtaggtttgccaataactctcgctcaactggatattaaagaagatgtcccggcgaaaatgcgaattgtggcagaagcggcatgtgcagaaggtgaaaccattcacaacatgcctggcggcgcgacgccagatcaggtttacgccgctctgctggtagccgaccagtacggtcagcgtttcctgcaagagtgggaataa

SEQ ID NO: 59 - protein, 367 aa, Artificial Sequence
  Synthesized: gldA Protein P0A9S5
MDRIIQSPGKYIQGADVINRLGEYLKPLAERWLVVGDKFVLGFAQSTVEKSFKDAGLVVEIAPFGGECSQNEIDRLRGIAETAQCGAILGIGGGKTLDTAKALAHFMGVPVAIAPTIASTDAPCSALSVIYTDEGEFDRYLLLPNNPNMVIVDTKIVAGAPARLLAAGIGDALATWFEARACSRSGATTMAGGKCTQAALALAELCYNTLLEEGEKAMLAAEQHVVTPALERVIEANTYLSGVGFESGGLAAAHAVHNGLTAIPDAHHYYHGEKVAFGTLTQLVLENAPVEEIETVAALSHAVGLPITLAQLDIKEDVPAKMRIVAEAACAEGETIHNMPGGATPDQVYAALLVADQYGQRFLQEWE

SEQ ID NO: 60 - DNA, 777 bp, Artificial Sequence
  Synthesized: gre2  854014
atgagcaaagttgcaatggttaccggtggtgcacaaggcatcggtcgtggaatttcagagaagctggcagcagatggtttcgatattgccgtagccgacctgccacaacaggaagaacaagctgcagagaccatcaagttgattgaagctgcagatcaaaaggctgtattcgttggattagatgtcaccgataaggctaatttcgacagtgcaattgatgaggcagcagagaaacttggcggcttcgatgtgctagtaaacaacgccggcatcgcacaaattaagccacttctggaagtcaccgaagaagacctaaagcagatctactccgtgaacgtttttagcgtattttttggtattcaagcagcatcccgaaagttcgatgagcttggcgtaaaaggcaagatcatcaacgctgcatcaatcgctgctatccaaggtttcccaatcttgagcgcctactccaccaccaaattcgcggtgcgtggcctcacccaggctgctgctcaagaacttgctccgaagggccacaccgtcaacgcatacgcacctggcatcgtgggcaccggaatgtgggagcagatcgatgctgaactttccaagatcaacggcaaaccaattggtgagaacttcaaggagtactcctcctcaatcgcattgggccgaccatcagtacctgaggatgtagccggactggtttctttcttggcgtctgaaaactccaactatgtcaccgggcaggtcatgcttgtcgacggcggcatgctctacaactaa

SEQ ID NO: 61 - protein, 342 aa, Artificial Sequence
  Synthesized: gre2 Protein Q12068
MSVFVSGANGFIAQHIVDLLLKEDYKVIGSARSQEKAENLTEAFGNNPKFSMEVVPDISKLDAFDHVFQKHGKDIKIVLHTASPFCFDITDSERDLLIPAVNGVKGILHSIKKYAADSVERVVLTSSYAAVFDMAKENDKSLTFNEESWNPATWESCQSDPVNAYCGSKKFAEKAAWEFLEENRDSVKFELTAVNPVYVFGPQMFDKDVKKHLNTSCELVNSLMHLSPEDKIPELFGGYIDVRDVAKAHLVAFQKRETIGQRLIVSEARFTMQDVLDILNEDFPVLKGNIPVGKPGSGATHNTLGATLDNKKSKKLLGFKFRNLKETIDDTASQILKFEGRI

SEQ ID NO: 62 - DNA, 984 bp, Artificial Sequence
  Synthesized: gre3 856504
atgtcttcactggttactcttaataacggtctgaaaatgcccctagtcggcttagggtgctggaaaattgacaaaaaagtctgtgcgaatcaaatttatgaagctatcaaattaggctaccgtttattcgatggtgcttgcgactacggcaacgaaaaggaagttggtgaaggtatcaggaaagccatctccgaaggtcttgtttctagaaaggatatatttgttgtttcaaagttatggaacaattttcaccatcctgatcatgtaaaattagctttaaagaagaccttaagcgatatgggacttgattatttagacctgtattatattcacttcccaatcgccttcaaatatgttccatttgaagagaaataccctccaggattctatacgggcgcagatgacgagaagaaaggtcacatcaccgaagcacatgtaccaatcatagatacgtaccgggctctggaagaatgtgttgatgaaggcttgattaagtctattggtgtttccaactttcagggaagcttgattcaagatttattacgtggttgtagaatcaagcccgtggctttgcaaattgaacaccatccttatttgactcaagaacacctagttgagttttgtaaattacacgatatccaagtagttgcttactcctccttcggtcctcaatcattcattgagatggacttacagttggcaaaaaccacgccaactctgttcgagaatgatgtaatcaagaaggtctcacaaaaccatccaggcagtaccacttcccaagtattgcttagatgggcaactcagagaggcattgccgtcattccaaaatcttccaagaaggaaaggttacttggcaacctagaaatcgaaaaaaagttcactttaacggagcaagaattgaaggatatttctgcactaaatgccaacatcagatttaatgatccatggacctggttggatggtaaattccccacttttgcctga

SEQ ID NO: 63 - protein, 327 aa, Artificial Sequence
  Synthesized: gre3 Protein P38715
MSSLVTLNNGLKMPLVGLGCWKIDKKVCANQIYEAIKLGYRLFDGACDYGNEKEVGEGIRKAISEGLVSRKDIFVVSKLWNNFHHPDHVKLALKKTLSDMGLDYLDLYYIHFPIAFKYVPFEEKYPPGFYTGADDEKKGHITEAHVPIIDTYRALEECVDEGLIKSIGVSNFQGSLIQDLLRGCRIKPVALQIEHHPYLTQEHLVEFCKLHDIQVVAYSSFGPQSFIEMDLQLAKTTPTLFENDVIKKVSQNHPGSTTSQVLLRWATQRGIAVIPKSSKKERLLGNLEIEKKFTLTEQELKDISALNANIRFNDPWTWLDGKFPTFA

SEQ ID NO: 64 - DNA, 777 bp, Artificial Sequence; identical to SEQ ID NO: 60
  Synthesized: budC 4062841

SEQ ID NO: 65 - protein, 258 aa, Artificial Sequence
  Synthesized: budC Protein Q9ZNN8
MSKVAMVTGGAQGIGRGISEKLAADGFDIAVADLPQQEEQAAETIKLIEAADQKAVFVGLDVTDKANFDSAIDEAAEKLGGFDVLVNNAGIAQIKPLLEVTEEDLKQIYSVNVFSVFFGIQAASRKFDELGVKGKIINAASIAAIQGFPILSAYSTTKFAVRGLTQAAAQELAPKGHTVNAYAPGIVGTGMWEQIDAELSKINGKPIGENFKEYSSSIALGRPSVPEDVAGLVSFLASENSNYVTGQVMLVDGGMLYN

SEQ ID NO: 66 - DNA, 1149 bp, Artificial Sequence
  Synthesized: bdh1 851239
atgagagctttggcatatttcaagaagggtgatattcacttcactaatgatatccctaggccagaaatccaaaccgacgatgaggttattatcgacgtctcttggtgtgggatttgtggctcggatcttcacgagtacttggatggtccaatcttcatgcctaaagatggagagtgccataaattatccaacgctgctttacctctggcaatgggccatgagatgtcaggaattgtttccaaggttggtcctaaagtgacaaaggtgaaggttggcgaccacgtggtcgttgatgctgccagcagttgtgcggacctgcattgctggccacactccaaattttacaattccaaaccatgtgatgcttgtcagaggggcagtgaaaatctatgtacccacgccggttttgtaggactaggtgtgatcagtggtggctttgctgaacaagtcgtagtctctcaacatcacattatcccggttccaaaggaaattcctctagatgtggctgctttagttgagcctctttctgtcacctggcatgctgttaagatttctggtttcaaaaaaggcagttcagccttggttcttggtgcaggtcccattgggttgtgtaccattttggtacttaagggaatgggggctagtaaaattgtagtgtctgaaattgcagagagaagaatagaaatggccaagaaactgggcgttgaggtgttcaatccctccaagcacggtcataaatctatagagatactacgtggtttgaccaagagccatgatgggtttgattacagttatgattgttctggtattcaagttactttcgaaacctctttgaaggcattaacattcaaggggacagccaccaacattgcagtttggggtccaaaacctgtcccattccaaccaatggatgtgactctccaagagaaagttatgactggttcgatcggctatgttgtcgaagacttcgaagaagttgttcgtgccatccacaacggagacatcgccatggaagattgtaagcaactaatcactggtaagcaaaggattgaggacggttgggaaaagggattccaagagttgatggatcacaaggaatccaacgttaagattctattgacgcctaacaatcacggtgaaatgaagtaa

SEQ ID NO: 67 - protein, 382 aa, Artificial Sequence
  Synthesized: bdh1 Protein P39714
MRALAYFKKGDIHFTNDIPRPEIQTDDEVIIDVSWCGICGSDLHEYLDGPIFMPKDGECHKLSNAALPLAMGHEMSGIVSKVGPKVTKVKVGDHVVVDAASSCADLHCWPHSKFYNSKPCDACQRGSENLCTHAGFVGLGVISGGFAEQVVVSQHHIIPVPKEIPLDVAALVEPLSVTWHAVKISGFKKGSSALVLGAGPIGLCTILVLKGMGASKIVVSEIAERRIEMAKKLGVEVFNPSKHGHKSIEILRGLTKSHDGFDYSYDCSGIQVTFETSLKALTFKGTATNIAVWGPKPVPFQPMDVTLQEKVMTGSIGYVVEDFEEVVRAIHNGDIAMEDCKQLITGKQRIEDGWEKGFQELMDHKESNVKILLTPNNHGEMK

SEQ ID NO: 68 - DNA, 1167 bp, Artificial Sequence
  Synthesized: yqhD*
atgaacaattttaatttgcatactccaactagaatattatttggaaaaggtgcaattgcaggtttaagggaacaaataccacatgatgcaagggtattaatcacatacggtggtggttctgtcaagaaaactggtgtattggatcaagtattggatgctttaaagggtatggatgtcttggaatttggaggaatcgaaccaaaccctgcttacgagactttaatgaatgctgtcaaattggtcagagaacaaaaggtaacattcttattggctgttggaggtggatcagtattagatggtacaaagttcattgctgctgcagcaaattatccagaaaacattgatccatggcatatattgcaaactggtggtaaggaaataaagtcagctatcccaatgggatgtgttttgacattgcctgcaacaggatcagaatcaaacgctgaagcagtcatctcaagaaagactacaggtgacaaacaggcattccattctgcccatgtccaacctgtatttgctgttttagaccctgtatacacttacacattaccaccaaggcaagtcgcaaatggagttgtcgatgcctttgttcacactgtagaacagtacgtcaccaaaccagtcgatgcaaagatccaggacaggtttgcagaaggtattttattgacattaatcgaagatggaccaaaagcattgaaagagccagagaactatgacgttagggcaaatgttatgtgggctgctacccaggcattgaacggtttaattggtgcaggagttccacaagattgggctacacacatgttgggtcacgagttgaccgccatgcacggtttggaccatgcacagactttagccattgttttgcctgccttatggaacgagaaaagagatactaagagggctaagttattacaatacgctgaaagggtttggaatatcaccgagggatctgatgatgaaaggattgatgccgctattgcagccactagaaacttctttgaacaattaggtgttccaactcacttgtctgactatggtttagatggatcatctattccagctttgttgaagaaattggaagagcacggtatgacccagttgggtgagaatcatgatataaccttagatgtatctaggagaatctacgaggctgctagataatga

SEQ ID NO: 69 - protein, 387 aa, Artificial Sequence
  Synthesized: yqhD* Protein Q46856
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAEAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 70 - DNA, 981 bp, Artificial Sequence
  Synthesized: ydjg 12930149
atgaaaaagatacctttaggcacaacggatattacgctttcgcgaatggggttggggacatgggccattggcggcggtcctgcatggaatggcgatctcgatcggcaaatatgtattgatacgattcttgaagcccatcgttgtggcattaatctgattgatactgcgccaggatataactttggcaatagtgaagttatcgtcggtcaggcgttaaaaaaactgccccgtgaacaggttgtagtagaaaccaaatgcggcattgtctgggaacgaaaaggaagtttattcaacaaagttggcgatcggcagttgtataaaaacctttccccggaatctatccgcgaagaggtagcagcgagcttgcaacgtctgggtattgattacatcgatatctacatgacgcactggcagtcggtgccgccattttttacgccgatcgctgaaactgtcgcagtgcttaatgagttaaagtctgaagggaaaattcgcgctataggcgctgctaacgtcgatgctgaccatatccgcgagtatctgcaatatggtgaactggatattattcaggcgaaatacagtatcctcgaccgggcaatggaaaacgaactgctgccactatgtcgtgataatggcattgtggttcaggtttattccccgctagagcagggattgttgaccggcaccatcactcgtgattacgttccgggcggcgctcgggcaaataaagtctggttccagcgtgaaaacatgctgaaagtgattgatatgcttgaacagtggcagccactttgtgctcgttatcagtgcacaattcccactctggcactggcgtggatattaaaacagagtgatttaatctccattcttagtggggctactgcaccggaacaggtacgcgaaaatgtcgcggcactgaatatcaacttatcggatgcagacgcaacattgatgagggaaatggcagaggccctggagcgttaa

SEQ ID NO: 71 - protein, 326 aa, Artificial Sequence
  Synthesized: ydjg Protein P77256
MKKIPLGTTDITLSRMGLGTWAIGGGPAWNGDLDRQICIDTILEAHRCGINLIDTAPGYNFGNSEVIVGQALKKLPREQVVVETKCGIVWERKGSLFNKVGDRQLYKNLSPESIREEVAASLQRLGIDYIDIYMTHWQSVPPFFTPIAETVAVLNELKSEGKIRAIGAANVDADHIREYLQYGELDIIQAKYSILDRAMENELLPLCRDNGIVVQVYSPLEQGLLTGTITRDYVPGGARANKVWFQRENMLKVIDMLEQWQPLCARYQCTIPTLALAWILKQSDLISILSGATAPEQVRENVAALNINLSDADATLMREMAEALER

SEQ ID NO: 72 - DNA, 939 bp, Artificial Sequence
  Synthesized: ypr1 256273055
atgcctgctacgttaaagaattcttctgctacattaaaactaaatactggtgcctccattccagtgttgggtttcggcacttggcgttccgttgacaatagcggttaccattctgtaattgcagctttgaaagctggatacagacacattgatgctgcggctatctatttgaatgaagaagaagttggcagggctattaaagattccggagtccctcgtgaggaaatttttattactactaagctttggggtacggaacaacgtgatccggaagctgctctaaacaagtctttgaaaagactaggcttggattatgttgacctatatctgatgcattggccagtgcctttgaaaaccgacagagttactgatggtaacgttctgtgtattccaacattagaagatggcactgttgacatcgatactaaggaatggaattttatcaagacgtgggagttgatgcaagaattgccaaagacgggcaaaactaaagccgttggtgtctctaatttttctattaacaacattaaagaattattagaatctccaaataacaaggtggtaccagctactaatcaaattgaaattcatccattgctaccacaagacgaattgattgccttttgtaaagaaaagggtatcgttgttgaagcctactcaccatttgggagtgctaatgctcctttactaaaagagcaagcaattattgatatggctaaaaagcacggtgttgagccagcacagcttattatcagttggagtattcaaagaggctacgttgttctggccaaatcggttaatcctgaaagaattgtatccaattttaagattttcactctgcccgaggatgatttcaagactattagtaacctatccaaagtgcatggtacaaagagagtcgttgatatgaagtggggatccttcccaattttccaatga

SEQ ID NO: 73 - protein, 312 aa, Artificial Sequence
  Synthesized: ypr1 Protein C7GMG9
MPATLKNSSATLKLNTGASIPVLGFGTWRSVDNSGYHSVIAALKAGYRHIDAAAIYLNEEEVGRAIKDSGVPREEIFITTKLWGTEQRDPEAALNKSLKRLGLDYVDLYLMHWPVPLKTDRVTDGNVLCIPTLEDGTVDIDTKEWNFIKTWELMQELPKTGKTKAVGVSNFSINNIKELLESPNNKVVPATNQIEIHPLLPQDELIAFCKEKGIVVEAYSPFGSANAPLLKEQAIIDMAKKHGVEPAQLIISWSIQRGYVVLAKSVNPERIVSNFKIFTLPEDDFKTISNLSKVHGTKRVVDMKWGSFPIFQ

SEQ ID NO: 74 - DNA, 771 bp, Artificial Sequence
  Synthesized: budC 3907615
atgaaaaaagtcgcacttgttaccggcgccggccaggggattggtaaagctatcgcccttcgtctggtgaaggatggatttgccgtggccattgccgattataacgacgccaccgccaaagcggtcgcctccgaaatcaaccaggccggcggccgcgccatggcggtgaaagtggatgtttctgaccgcgaccaggtatttgccgccgtcgaacaggcgcgcaaaacgctgggcggcttcgacgtcatcgtcaacaacgccggcgtggcgccgtccacgccgatcgagtccattaccccggagattgtcgacaaagtctacaacatcaacgtcaaaggggtgatctggggcatccaggcggcggtcgaggcctttaagaaagagggtcacggcgggaaaatcatcaacgcctgttcccaggccggccacgtcggtaacccggagctggcggtgtatagctcgagtaaattcgccgtacgcggcttaacccagaccgccgctcgcgacctcgcgccgctgggcatcacggtcaacggctactgcccggggattgtcaaaacgccaatgtgggccgaaattgaccgccaggtgtccgaagccgccggtaaaccgctgggctacggtaccgccgagttcgccaaacgcatcactctcggtcgtctgtccgagccggaagatgtcgccgcctgcgtctcctatcttgccagcccggattctgattacatgaccggtcagtcgttgctgatcgacggcgggatggtatttaactaa

SEQ ID NO: 75 - protein, 256 aa, Artificial Sequence
  Synthesized: budC Protein Q48436
MKKVALVTGAGQGIGKAIALRLVKDGFAVAIADYNDATAKAVASEINQAGGRAMAVKVDVSDRDQVFAAVEQARKTLGGFDVIVNNAGVAPSTPIESITPEIVDKVYNINVKGVIWGIQAAVEAFKKEGHGGKIINACSQAGHVGNPELAVYSSSKFAVRGLTQTAARDLAPLGITVNGYCPGIVKTPMWAEIDRQVSEAAGKPLGYGTAEFAKRITLGRLSEPEDVAACVSYLASPDSDYMTGQSLLIDGGMVFN

SEQ ID NO: 76 - DNA, 1149 bp, Artificial Sequence
  Synthesized: fucO 146044
atggctaacagaatgattctgaacgaaacggcatggtttggtcggggtgctgttggggctttaaccgatgaggtgaaacgccgtggttatcagaaggcgctgatcgtcaccgataaaacgctggtgcaatgcggcgtggtggcgaaagtgaccgataagatggatgctgcagggctggcatgggcgatttacgacggcgtagtgcccaacccaacaattactgtcgtcaaagaagggctcggtgtattccagaatagcggcgcggattacctgatcgctattggtggtggttctccacaggatacttgtaaagcgattggcattatcagcaacaacccggagtttgccgatgtgcgtagcctggaagggctttccccgaccaataaacccagtgtaccgattctggcaattcctaccacagcaggtactgcggcagaagtgaccattaactacgtgatcactgacgaagagaaacggcgcaagtttgtttgcgttgatccgcatgatatcccgcaggtggcgtttattgacgctgacatgatggatggtatgcctccagcgctgaaagctgcgacgggtgtcgatgcgctcactcatgctattgaggggtatattacccgtggcgcgtgggcgctaaccgatgcactgcacattaaagcgattgaaatcattgctggggcgctgcgaggatcggttgctggtgataaggatgccggagaagaaatggcgctcgggcagtatgttgcgggtatgggcttctcgaatgttgggttagggttggtgcatggtatggcgcatccactgggcgcgttttataacactccacacggtgttgcgaacgccatcctgttaccgcatgtcatgcgttataacgctgactttaccggtgagaagtaccgcgatatcgcgcgcgttatgggcgtgaaagtggaaggtatgagcctggaagaggcgcgtaatgccgctgttgaagcggtgtttgctctcaaccgtgatgtcggtattccgccacatttgcgtgatgttggtgtacgcaaggaagacattccggcactggcgcaggcggcactggatgatgtttgtaccggtggcaacccgcgtgaagcaacgcttgaggatattgtagagctttaccataccgcctggtaa

SEQ ID NO: 77 - protein, 382 aa, Artificial Sequence
  Synthesized: fucO Protein P0A9S1
MANRMILNETAWFGRGAVGALTDEVKRRGYQKALIVTDKTLVQCGVVAKVTDKMDAAGLAWAIYDGVVPNPTITVVKEGLGVFQNSGADYLIAIGGGSPQDTCKAIGIISNNPEFADVRSLEGLSPTNKPSVPILAIPTTAGTAAEVTINYVITDEEKRRKFVCVDPHDIPQVAFIDADMMDGMPPALKAATGVDALTHAIEGYITRGAWALTDALHIKAIEIIAGALRGSVAGDKDAGEEMALGQYVAGMGFSNVGLGLVHGMAHPLGAFYNTPHGVANAILLPHVMRYNADFTGEKYRDIARVMGVKVEGMSLEEARNAAVEAVFALNRDVGIPPHLRDVGVRKEDIPALAQAALDDVCTGGNPREATLEDIVELYHTAW

SEQ ID NO: 78 - DNA, 804 bp, Artificial Sequence
  Synthesized: yafB 545778205
atggctatccctgcatttggtttaggtactttccgtctgaaagacgacgttgttatttcatctgtgataacggcgcttgaacttggttatcgcgcaattgataccgcacaaatctatgataacgaagccgcagtaggtcaggcgattgcagaaagtggcgtgccacgtcatgaactctacatcaccactaaaatctggattgaaaatctcagcaaagacaaattgatcccaagtctgaaagagagcctgcaaaaattgcgtaccgattatgttgatctgacgctaatccactggccgtcaccaaacgatgaagtctctgttgaagagtttatgcaggcgctgctggaagccaaaaaacaagggctgacgcgtgagatcggtatttccaacttcacgatcccgttgatggaaaaagcgattgctgctgttggtgctgaaaacatcgctactaaccagattgaactctctccttatctgcaaaaccgtaaagtggttgcctgggctaaacagcacggcatccatattacttcctatatgacgctggcgtatggtaaggccctgaaagatgaggttattgctcgtatcgcagctaaacacaatgcgactccggcacaagtgattctggcgtgggctatgggggaaggttactcagtaattccttcttctactaaacgtaaaaacctggaaagtaatcttaaggcacaaaatttacagcttgatgccgaagataaaaaagcgatcgccgcactggattgcaacgaccgcctggttagcccggaaggtctggctcctgaatgggattaa

SEQ ID NO: 79 - protein, 267 aa, Artificial Sequence
  Synthesized: yafB Protein P30863
MAIPAFGLGTFRLKDDVVISSVITALELGYRAIDTAQIYDNEAAVGQAIAESGVPRHELYITTKIWIENLSKDKLIPSLKESLQKLRTDYVDLTLIHWPSPNDEVSVEEFMQALLEAKKQGLTREIGISNFTIPLMEKAIAAVGAENIATNQIELSPYLQNRKVVAWAKQHGIHITSYMTLAYGKALKDEVIARIAAKHNATPAQVILAWAMGEGYSVIPSSTKRKNLESNLKAQNLQLDAEDKKAIAALDCNDRLVSPEGLAPEWD

SEQ ID NO: 80 - DNA, 2364 bp, Artificial Sequence
  Synthesized: dhaB1 384086948
atgataagtaaaggatttagtacccaaacagaaagaataaatattttaaaggctcaaatattaaatgctaaaccatgtgttgaatcagaaagagcaatattaataacagaatcatttaaacaaacagaaggccagccagcaattttaagaagagcattggcattgaaacacatacttgaaaatatccctataacaattagagatcaagaacttatagtgggaagtttaactaaagaaccaaggtcttcacaagtatttcctgagttttctaataagtggttacaagatgaattggatagattaaataagagaactggagatgcattccaaatttcagaagaaagtaaagaaaaattaaaagatgtctttgagtattggaatggaaagacaacaagtgagttagcaacttcatatatgacagaggaaacaagagaggcagtaaattgtgatgtatttactgtaggaaactactattataatggcgtaggacatgtatctgtagattatggaaaagtattaagggttggatttaatgggattataaatgaggctaaggaacaattagaaaaaaacaggagtatagatcctgattttataaagaaagaaaaattcctaaatagtgttattatctcatgcgaagctgcaataacatatgtaaatagatatgctaaaaaggctaaagagattgcagataatacaagtgatgcaaaaagaaaagctgaattaaatgaaatagcaaaaatttgttcaaaagtttcaggagagggagctaaatctttctatgaagcatgtcaattattttggtttattcatgcaataataaatatagaatctaatggacattctatttctccagctagatttgatcaatacatgtatccatattatgaaaatgataaaaatataacagataagtttgctcaagaattaatagattgtatctggattaaattaaatgatattaataaagtaagagatgagatttcaactaaacattttggtggttacccaatgtatcaaaacttaattgttgggggtcaaaattcagaaggaaaagatgcaactaataaagtatcatatatggcattagaagcagctgtccatgtaaagttgcctcagccatctttgtcagtaagaatatggaataagactccagatgaatttttgcttagagcagcagaattaactagagaagggttaggacttcctgcttattataatgatgaagttattattccagcattagtttctagaggtcttacattagaagatgcaagagactacggaataattggatgtgttgaaccacaaaagccaggaaaaacagaaggatggcatgattcagcattctttaatcttgcaagaatagtagagttaactataaattctggatttgataaaaataaacagattggacctaaaactcaaaattttgaagaaatgaaatcctttgatgaattcatgaaagcttataaagctcaaatggagtattttgtaaaacatatgtgctgtgctgataattgcatagatattgcacatgcagaaagagctccattacctttcttgtcatcaatggttgataattgtatcggaaaaggaaagagccttcaagatggtggtgcagaatataacttcagtggaccacaaggtgttggagtagctaatattggagattcattagttgcagttaaaaaaattgtgtttgatgaaaataagattactccttcagaattaaagaaaacattaaataatgattttaaaaattcagaagaaatacaagccttactaaaaaatgctcctaagtttggaaatgatattgatgaagttgataatttagctagagagggtgcattagtatactgtagagaagttaataaatatacaaatccaaggggaggaaattttcaaccaggattatatccatcttcaattaatgtatattttggaagcttaacaggtgctactccagatggaaggaaatccggacaaccattagctgatggggtttctccatcaagaggctgtgatgtatctggacctactgcagcttgtaactcagttagtaaattagatcattttatagcttcaaatggaactttatttaatcaaaaattccatccgtcagcattaaaaggtgataatggattaatgaatttatcatcattaataagaagttattttgatcaaaagggatttcatgttcaatttaatgtaatagataaaaaaatattacttgcagcacaaaaaaatcctgaaaaatatcaagatttaattgttagagttgcaggatatagtgcacagttcatttctttagataaatctattcaaaatgatattattgcaagaactgaacatgttatgtaa

SEQ ID NO: 81 - protein, 787 aa, Artificial Sequence
  Synthesized: dhaB1 Protein Q8GEZ8
MISKGFSTQTERINILKAQILNAKPCVESERAILITESFKQTEGQPAILRRALALKHILENIPITIRDQELIVGSLTKEPRSSQVFPEFSNKWLQDELDRLNKRTGDAFQISEESKEKLKDVFEYWNGKTTSELATSYMTEETREAVNCDVFTVGNYYYNGVGHVSVDYGKVLRVGFNGIINEAKEQLEKNRSIDPDFIKKEKFLNSVIISCEAAITYVNRYAKKAKEIADNTSDAKRKAELNEIAKICSKVSGEGAKSFYEACQLFWFIHAIINIESNGHSISPARFDQYMYPYYENDKNITDKFAQELIDCIWIKLNDINKVRDEISTKHFGGYPMYQNLIVGGQNSEGKDATNKVSYMALEAAVHVKLPQPSLSVRIWNKTPDEFLLRAAELTREGLGLPAYYNDEVIIPALVSRGLTLEDARDYGIIGCVEPQKPGKTEGWHDSAFFNLARIVELTINSGFDKNKQIGPKTQNFEEMKSFDEFMKAYKAQMEYFVKHMCCADNCIDIAHAERAPLPFLSSMVDNCIGKGKSLQDGGAEYNFSGPQGVGVANIGDSLVAVKKIVFDENKITPSELKKTLNNDFKNSEEIQALLKNAPKFGNDIDEVDNLAREGALVYCREVNKYTNPRGGNFQPGLYPSSINVYFGSLTGATPDGRKSGQPLADGVSPSRGCDVSGPTAACNSVSKLDHFIASNGTLFNQKFHPSALKGDNGLMNLSSLIRSYFDQKGFHVQFNVIDKKILLAAQKNPEKYQDLIVRVAGYSAQFISLDKSIQNDIIARTEHVM

SEQ ID NO: 82 - DNA, 915 bp, Artificial Sequence
  Synthesized: dhaB2 384086948
atgagtaaggagataaaaggcgttttatttaacatacaaaaattttcgttacatgatgggcctggaataagaactatagtattttttaagggatgttcaatgtcgtgcttatggtgcagtaatccagaatcccaagatattaaacctcaagtaatgtttaataaaaatttatgtacaaaatgtggaagatgtaaatctcaatgtaaaagtgcagctattgatatgaattcagaatataggatagataaaagcaaatgtacagagtgtacaaaatgtgttgataattgcttaagcggggcacttgttattgaaggaaggaattacagtgttgaagacgttataaaggaattgaaaaaagatagtgttcaatatagaagatcaaacggtggaattacactatctggaggggaagtattacttcaaccagattttgcagtggagcttttaaaagagtgtaaatcatatggctggcacactgccattgaaacagcaatgtatgttaatagtgaatctgtaaaaaaagtaattccatatatagatctggctatgattgatataaaaagtatgaatgatgaaatccataggaaatttacaggagtgagtaacgaaataatattacaaaacattaaattaagtgatgaattagctaaagaaataataatcagaattcctgtaatagaaggatttaatgcagatttacaaagtataggagcaatagctcaattttcaaaatcattaacaaatcttaaaagaatagatcttcttccataccataattatggagaaaataagtatcaagcaattggaagagagtattctttgaaagaactaaaatcacctagtaaagacaaaatggaaagattaaaagctttagttgaaatcatgggaataccgtgcacaattggagctgagtaa

SEQ ID NO: 83 - protein, 304 aa, Artificial Sequence
  Synthesized: dhaB2 Protein Q8GEZ7
MSKEIKGVLFNIQKFSLHDGPGIRTIVFFKGCSMSCLWCSNPESQDIKPQVMFNKNLCTKCGRCKSQCKSAAIDMNSEYRIDKSKCTECTKCVDNCLSGALVIEGRNYSVEDVIKELKKDSVQYRRSNGGITLSGGEVLLQPDFAVELLKECKSYGWHTAIETAMYVNSESVKKVIPYIDLAMIDIKSMNDEIHRKFTGVSNEIILQNIKLSDELAKEIIIRIPVIEGFNADLQSIGAIAQFSKSLTNLKRIDLLPYHNYGENKYQAIGREYSLKELKSPSKDKMERLKALVEIMGIPCTIGAE

SEQ ID NO: 84 - DNA, 2532 bp, Artificial Sequence
  Synthesized: b1 83596364
atgggaaattatgatagtactccaattgcgaagtcggatcgtataaaaagacttgtagatcatctgtatgcaaagatgcctgagattgaggcggcaagagcggaactgatcacagaatcatttaaggctacggaaggtcagccggtagtgatgcgcaaagcacgtgcttttgaacatattttaaagaatcttccgatcattatcagaccagaagaattaattgtcggaagtacaacgatcgcaccgagaggatgccagacatatccggaattttcatatgaatggttagaggcagaattcgaaacagtcgaaacaagaagtgctgatccattctatatttcagaggaaacaaaaaagagattattagctgcagatgcttactggaaaggaaaaacaaccagtgagctggcaacttcctatatggctccggagacactccgtgccatgaaacataatttctttacaccgggcaactatttttataatggtgtaggacatgtaacagttcagtatgaaaccgtattggcgatcggtctgaatggtgtaaaagaaaaagtcagaaaagagatggagaactgccattttggagatgcggattattctaccaagatgtgtttcttagaatccatcctgatttcctgtgatgcagtcatcacttatgcaaatcgttatgcgaaaatggcagaagagatggcagagaaagaaacagatgcagcaagaagacaggagcttctgacaattgcaagagtatgtaaaaatgtaccggaattccctgctgaaagcttccaggaggcgtgccagtccttctggttcatccagcaggtattacagattgaatccagtggacattctatttcaccgggacgttttgaccagtatatgtatccttattacgagaaggatttaaaagaaggcagtctcacccgtgagtacgcacaggaactgatcgactgtatctgggtaaaattaaatgatctgaataaatgtcgtgatgccgcaagtgcagaaggttttgcaggatattccttattccagaacctgatcgttggtggacagacagttcagggaagagacgctaccaatgatctttcgtttatgtgcatcactgccagtgagcatgtatttttaccaatgccatccttatcgatccgtgtgtggcatggatcatccaaggcattattaatgcgtgcggcagagctgacaagaaccggtatcggtttaccggcttattataatgacgaagttatcattcctgcattggttcatcgtggagcaaccatggacgaggcaaggaattacaacatcatcggatgtgtagaaccgcaggttccgggtaaaacagacggatggcacgatgcagcgttcttcaatatgtgccgcccattggagatggtattttccaatggttatgacaatggagagatcgcaagtatccagaccggtaatgtggagagcttccagtcatttgatgaatttatggaagcatacagaaaacagatgttatataacatcgaattgatggtaaatgcagataatgcaattgattatgctcatgcaaagcttgcaccattaccatttgagtcatgtctggtagatgactgcatcaagcggggaatgagtgcacaggaaggcggagcaatttataactttaccggtccgcagggctttggtatcgcaaatgtcgcagactctttatatacgatcaagaagctggtatttgaagaaaaacgcattaccatgggcgagttaaagaaagctcttgagatgaattacggtaaagggctggatgccacaactgccggagatattgcaatgcaggttgcaaaaggattaaaagatgcaggtcaggaagtgggacctgatgtgatagcgaatacgatcagacaggtattagagatggaattaccggaagatgtcaggaagcgttatgaagagatccatgaaatgatccttgaacttccgaaatacggaaatgatattgatgaagtagatgagcttgcccgcgaggcagcatatttctacacaagaccattagagacattcaaaaatccaagaggtggaatgtatcaggcaggtctctatccggtatcagccaatgttccattaggagctcagaccggtgctactccggacggaagattagcacatactccggtggcagatggagtcggaccgacatcaggattcgatatcagtggaccgacagcatcctgtaactcagttgcaaaattagatcatgcgatcgcaagtaacggaacactctttaatatgaaaatgcatccaacagctatggctggagagaaggggctggagagctttatttctctgattcgtggttactttgatcagcagggtatgcacatgcagtttaatgtcgtagaccgtgcaactcttttggacgcacaggctcatccagaaaaatacagtgggctgatcgtacgtgtagccggatattctgctttgtttactacgttatcgaaatccttacaggatgatatcattaagagaacagaacaggctgataatcgatag

SEQ ID NO: 85 - protein, 843 aa, Artificial Sequence
  Synthesized: b1 Protein Q1A666
MGNYDSTPIAKSDRIKRLVDHLYAKMPEIEAARAELITESFKATEGQPVVMRKARAFEHILKNLPIIIRPEELIVGSTTIAPRGCQTYPEFSYEWLEAEFETVETRSADPFYISEETKKRLLAADAYWKGKTTSELATSYMAPETLRAMKHNFFTPGNYFYNGVGHVTVQYETVLAIGLNGVKEKVRKEMENCHFGDADYSTKMCFLESILISCDAVITYANRYAKMAEEMAEKETDAARRQELLTIARVCKNVPEFPAESFQEACQSFWFIQQVLQIESSGHSISPGRFDQYMYPYYEKDLKEGSLTREYAQELIDCIWVKLNDLNKCRDAASAEGFAGYSLFQNLIVGGQTVQGRDATNDLSFMCITASEHVFLPMPSLSIRVWHGSSKALLMRAAELTRTGIGLPAYYNDEVIIPALVHRGATMDEARNYNIIGCVEPQVPGKTDGWHDAAFFNMCRPLEMVFSNGYDNGEIASIQTGNVESFQSFDEFMEAYRKQMLYNIELMVNADNAIDYAHAKLAPLPFESCLVDDCIKRGMSAQEGGAIYNFTGPQGFGIANVADSLYTIKKLVFEEKRITMGELKKALEMNYGKGLDATTAGDIAMQVAKGLKDAGQEVGPDVIANTIRQVLEMELPEDVRKRYEEIHEMILELPKYGNDIDEVDELAREAAYFYTRPLETFKNPRGGMYQAGLYPVSANVPLGAQTGATPDGRLAHTPVADGVGPTSGFDISGPTASCNSVAKLDHAIASNGTLFNMKMHPTAMAGEKGLESFISLIRGYFDQQGMHMQFNVVDRATLLDAQAHPEKYSGLIVRVAGYSALFTTLSKSLQDDIIKRTEQADNR

SEQ ID NO: 86 - DNA, 795 bp, Artificial Sequence
  Synthesized: b2 83596364
atgaaagaatatttgaatacatccggcaggatttttgatattcaaagatattccatacatgatggtccgggagtccgaaccatagtcttcttaaaaggatgtgcgttacgatgcagatggtgctgtaatccggaatcacagtcttttgaagtggaaacaatgacgatcaacggaaaaccaaaggttatgggcaaagatgtaactgtagcggaggttatgaagacagtagaaagagacatgccttattatttacagtccggtggaggaatcactctttccggtggtgaatgtacgcttcaaccggagttctcattagggcttttaagagcagcaaaagatttgggaatatcaacagccattgaaagtatggcttatgcaaaatacgaagtgatcgaaacactgcttccgtatctggatacttacttaatggatattaagcatatgaatccggaaaagcataaagaatatacaggtcatgataatctcagaatgttggagaatgcactcagggtagcccacagtgggcagacagaactgatcatccgtgttcctgttattcctggatttaatgctacagagcaggaattgcttgatatagcgaagtttgcagataccttaccgggcgtcagacagatccacatattgccttatcataactttggtcagggaaaatacgaaggattgaacagagactatccaatgggagatacagagaagccttccaatgagcagatgaaggcatttcaggaaatgatccaaaaaaatacgtcattacactgccagattggtggttaa

SEQ ID NO: 87 - protein, 264 aa, Artificial Sequence
  Synthesized: b2  Protein Q1A665
MKEYLNTSGRIFDIQRYSIHDGPGVRTIVFLKGCALRCRWCCNPESQSFEVETMTINGKPKVMGKDVTVAEVMKTVERDMPYYLQSGGGITLSGGECTLQPEFSLGLLRAAKDLGISTAIESMAYAKYEVIETLLPYLDTYLMDIKHMNPEKHKEYTGHDNLRMLENALRVAHSGQTELIIRVPVIPGFNATEQELLDIAKFADTLPGVRQIHILPYHNFGQGKYEGLNRDYPMGDTEKPSNEQMKAFQEMIQKNTSLHCQIGG

SEQ ID NO: 88 - DNA, 2580 bp, Artificial Sequence
  Synthesized: adh 308066805
atgaaggtaactaatgttgaagaactgatgaaaaaaatgcaggaagtgcaaaatgctcaaaaaaaatttgggagttttactcaggaacaagtagatgaaattttcaggcaagcagcactagcagctaacagtgccagaatagatctagctaaaatggcagtggaagaaactaaaatgggaattgtagaggataaggttataaaaaatcattttgttgcagaatacatatataataagtataaaaatgaaaaaacttgtgggattttggaagaagatgaaggctttggaatggttaaaattgcagaacctgtaggtgtgattgcagcagtaattccaacaacaaatccaacatctacagcaatatttaaagcattattagctttgaaaacaagaaatggtataattttttcaccacatccaagagcaaaaaagtgtactattgcagcagctaagttagttcttgatgctgcagttaaagcaggtgctcctaaaggaattataggttggatagatgaaccttctattgaactttcacagatagtaatgaaagaagctgatataatccttgcaacaggtggtccaggtatggttaaagcagcttattcttcaggtaaacctgctataggggttggtcctggtaacacacctgctttaattgatgaaagtgctgatattaaaatggcagtaaattcaatacttctttccaaaacttttgataatggtatgatttgtgcttcagagcagtcggtagtagttgtagattcaatatatgaagaagttaagaaagaatttgctcatagaggagcttatattttaagtaaggatgaaacaactaaagttggaaaaatactcttagttaatggtacattaaatgctggtatcgttggtcagagtgcttataaaatagcagaaatggcaggagttaaagttccagaagatgctaaagttcttataggagaagtaaaatcagtggagcattcagaagagccattttcacatgaaaagttatctccagttttagctatgtatagagctaaaaattttgatgaagctcttttaaaagctggaagattagttgaactcggtggaatgggtcatacatctgtattatatgtaaatgcaataactgaaaaagtaaaagtagaaaaatttagagaaactatgaagactggtagaacattaataaatatgccttcagcacaaggtgctataggagacatatataactttaaactagctccttcattaacattaggttgtggttcatggggaggaaactccgtatcagaaaatgttggacctaaacacttattaaatataaaaagtgttgctgagaggagagaaaatatgctttggtttagagttcctgaaaaggtttattttaaatatggtagtcttggagttgcattaaaagaattagatattttggataagaaaaaagtatttatagtaacagataaagttctttatcaattaggttatatagatagagttacaaagattcttgaagaattgaaaatttcatataaaatatttacagatgtagaaccagatccaaccctagctacagctaaaaaaggtgcagaagaattgttatcatttaatccagatactattatagcagttggtggtggttcagcaatggatgctgctaagattatgtgggtaatgtatgaacatccggaagtaagatttgaagatttagctatgagatttatggatataagaaagagagtatatacttttcctaagatgggtgaaaaagcaatgatgatttctgttgcaacatcagcaggaacaggatcagaagtaacaccttttgcagtaattactgatgaaaaaacaggagctaaatatccattagctgattatgaattaactccaaatatggctataattgatgctgaacttatgatgggtatgccaaaaggattaacagcagcttcaggaatagatgcactaactcatgcaatagaagcttatgtatcaataatggcttcagaatatactaatggattagcgttagaagcaataagattgatatttaagtatttaccaatagcttacagtgaaggaacaacaagtataaaggcaagagaaaaaatggcgcatgcttcaacaatagctggtatggcatttgctaatgcatttttaggagtatgtcattcaatggcacataaattaggatcaactcatcacgtaccacatggcattgccaatgcactacttataaatgaagttataaaatttaatgcagtagaaaatccaagaaaacaagctgcatttccacaatataagtatccaaatataaaaaagagatatgctagaatagcagattaccttaacttaggtgggtcaacagacgatgaaaaagtacaattattaataaatgctatagatgaattaaaagctaagataaatattccagaaagtattaaagaagcaggagtaacagaagaaaaattttatgctactttagataaaatgtcagaattagcttttgatgatcaatgtacaggtgcaaaccctagatatccattaataagtgaaataaaacaaatgtatgtaaatgcattttaa

SEQ ID NO: 89 - protein, 859 aa, Artificial Sequence
  Synthesized: adh Protein C6PZV5
MKVTNVEELMKKMQEVQNAQKKFGSFTQEQVDEIFRQAALAANSARIDLAKMAVEETKMGIVEDKVIKNHFVAEYIYNKYKNEKTCGILEEDEGFGMVKIAEPVGVIAAVIPTTNPTSTAIFKALLALKTRNGIIFSPHPRAKKCTIAAAKLVLDAAVKAGAPKGIIGWIDEPSIELSQIVMKEADIILATGGPGMVKAAYSSGKPAIGVGPGNTPALIDESADIKMAVNSILLSKTFDNGMICASEQSVVVVDSIYEEVKKEFAHRGAYILSKDETTKVGKILLVNGTLNAGIVGQSAYKIAEMAGVKVPEDAKVLIGEVKSVEHSEEPFSHEKLSPVLAMYRAKNFDEALLKAGRLVELGGMGHTSVLYVNAITEKVKVEKFRETMKTGRTLINMPSAQGAIGDIYNFKLAPSLTLGCGSWGGNSVSENVGPKHLLNIKSVAERRENMLWFRVPEKVYFKYGSLGVALKELDILDKKKVFIVTDKVLYQLGYIDRVTKILEELKISYKIFTDVEPDPTLATAKKGAEELLSFNPDTIIAVGGGSAMDAAKIMWVMYEHPEVRFEDLAMRFMDIRKRVYTFPKMGEKAMMISVATSAGTGSEVTPFAVITDEKTGAKYPLADYELTPNMAIIDAELMMGMPKGLTAASGIDALTHAIEAYVSIMASEYTNGLALEAIRLIFKYLPIAYSEGTTSIKAREKMAHASTIAGMAFANAFLGVCHSMAHKLGSTHHVPHGIANALLINEVIKFNAVENPRKQAAFPQYKYPNIKKRYARIADYLNLGGSTDDEKVQLLINAIDELKAKINIPESIKEAGVTEEKFYATLDKMSELAFDDQCTGANPRYPLISEIKQMYVNAF

SEQ ID NO: 90 - DNA, 990 bp, Artificial Sequence
  Synthesized: IdhA 946315
atgaaactcgccgtttatagcacaaaacagtacgacaagaagtacctgcaacaggtgaacgagtcctttggctttgagctggaattttttgactttctgctgacggaaaaaaccgctaaaactgccaatggctgcgaagcggtatgtattttcgtaaacgatgacggcagccgcccggtgctggaagagctgaaaaagcacggcgttaaatatatcgccctgcgctgtgccggtttcaataacgtcgaccttgacgcggcaaaagaactggggctgaaagtagtccgtgttccagcctatgatccagaggccgttgctgaacacgccatcggtatgatgatgacgctgaaccgccgtattcaccgcgcgtatcagcgtacccgtgatgctaacttctctctggaaggtctgaccggctttactatgtatggcaaaacggcaggcgttatcggtaccggtaaaatcggtgtggcgatgctgcgcattctgaaaggttttggtatgcgtctgctggcgttcgatccgtatccaagtgcagcggcgctggaactcggtgtggagtatgtcgatctgccaaccctgttctctgaatcagacgttatctctctgcactgcccgctgacaccggaaaactatcatctgttgaacgaagccgccttcgaacagatgaaaaatggcgtgatgatcgtcaataccagtcgcggtgcattgattgattctcaggcagcaattgaagcgctgaaaaatcagaaaattggttcgttgggtatggacgtgtatgagaacgaacgcgatctattctttgaagataaatccaacgacgtgatccaggatgacgtattccgtcgcctgtctgcctgccacaacgtgctgtttaccgggcaccaggcattcctgacagcagaagcactgaccagtatttctcagactacgctgcaaaacttaagcaatctggaaaaaggcgaaacctgcccgaacgaactggtttaa

SEQ ID NO: 91 - protein, 329 aa, Artificial Sequence
  Synthesized: IdhA Protein P52643
MKLAVYSTKQYDKKYLQQVNESFGFELEFFDFLLTEKTAKTANGCEAVCIFVNDDGSRPVLEELKKHGVKYIALRCAGFNNVDLDAAKELGLKVVRVPAYDPEAVAEHAIGMMMTLNRRIHRAYQRTRDANFSLEGLTGFTMYGKTAGVIGTGKIGVAMLRILKGFGMRLLAFDPYPSAAALELGVEYVDLPTLFSESDVISLHCPLTPENYHLLNEAAFEQMKNGVMIVNTSRGALIDSQAAIEALKNQKIGSLGMDVYENERDLFFEDKSNDVIQDDVFRRLSACHNVLFTGHQAFLTAEALTSISQTTLQNLSNLEKGETCPNELV

SEQ ID NO: 92 - DNA, 930 bp, Artificial Sequence
  Synthesized: IdhL2 1063343
atggataagaagcaacgcaaagtcgtaattgttggtgatggctcggtgggttcatcatttgccttttcattggtccaaaattgcgccctagatgaactcgttatcgttgacttggttaaaacgcacgcagagggggacgttaaggatttggaagatgttgccgcctttacgaatgcgaccaacattcataccggtgaatatgcggatgcgcgtgatgctgacatcgttgtcattacggctggtgtgcctcgtaagcctggtgagagtcgtttagatttgattaaccgcaatacgaagattctggaatccatcgtcaaaccagtggttgcgagtggttttaatggttgcttcgttatctcaagtaatcccgtcgatattttgacttcgatgacgcaacgtttatccggttttccacggcatcgggtcattggtaccgggacttccttggatacggcgcggttacgggtcgccttggctcagaagttgaatgttgccaccactgcagttgatgctgcggtacttggagaacatggtgatagttccatcgttaattttgatgaaattatgatcaatgctcagcccttaaagacggtcacaacggtcgatgatcagttcaaagctgaaatcgagcaagctgttcgtggtaaaggtggtcaaatcattagtcagaagggggccacgttctatggggtcgccgttagtttgatgcaaatctgccgagcaattttgaacgatgaaaatgctgagttgattgtctccgccgctttgtctggtcaatatggcattaacgatttgtacttggggtcacccgccattattaaccgcaacgggctccaaaaagtgatcgaagctgagctatcagatgatgagcgtgcccggatgcaacatttcgcagccaagatgctgaccatgatgaatgtggcatcataa

SEQ ID NO: 93 - protein, 309 aa, Artificial Sequence
  Synthesized: IdhL2 Protein P59390
MDKKQRKVVIVGDGSVGSSFAFSLVQNCALDELVIVDLVKTHAEGDVKDLEDVAAFTNATNIHTGEYADARDADIVVITAGVPRKPGESRLDLINRNTKILESIVKPVVASGFNGCFVISSNPVDILTSMTQRLSGFPRHRVIGTGTSLDTARLRVALAQKLNVATTAVDAAVLGEHGDSSIVNFDEIMINAQPLKTVTTVDDQFKAEIEQAVRGKGGQIISQKGATFYGVAVSLMQICRAILNDENAELIVSAALSGQYGINDLYLGSPAIINRNGLQKVIEAELSDDERARMQHFAAKMLTMMNVAS

SEQ ID NO: 94 - DNA, 999 bp, Artificial Sequence
  Synthesized: Idh2 217591
atggcaactctcaaggatcagctgattcagaatcttcttaaggaagaacatgtcccccagaataagattacaattgttggggttggtgctgttggcatggcctgtgccatcagtatcttaatgaaggacttggcagatgaagttgctcttgttgatgtcatggaagataaactgaagggagagatgatggatctccaacatggcagccttttccttagaacaccaaaaattgtctctggcaaagactataatgtgacagcaaactccaggctggttattatcacagctggggcacgtcagcaagagggagagagccgtctgaatttggtccagcgtaacgtgaacatctttaaattcatcattcctaatattgtaaaatacagcccaaattgcaagttgcttgttgtttccaatccagtcgatattttgacctatgtggcttggaagataagtggctttcccaaaaaccgtgttattggaagtggttgcaatctggattcagctcgcttccgttatctcatgggggagaggctgggagttcacccattaagctgccatgggtggatccttggggagcatggtgactctagtgtgcctgtatggagtggagtgaatgttgctggtgtctccctgaagaatttacaccctgaattaggcactgatgcagataaggaacagtggaaagcggttcacaaacaagtggttgacagtgcttatgaggtgatcaaactgaaaggctacacatcctgggccattggactgtcagtggccgatttggcagaaagtataatgaagaatcttaggcgggtgcatccgatttccaccatgattaagggtctctatggaataaaagaggatgtcttccttagtgttccttgcatcttgggacagaatggaatctcagacgttgtgaaagtgactctgactcatgaagaagaggcctgtttgaagaagagtgcagatacactttgggggatccagaaagaactgcagttttaa

SEQ ID NO: 95 - protein, 332 aa, Artificial Sequence
  Synthesized: Idh2 Protein P19858
MATLKDQLIQNLLKEEHVPQNKITIVGVGAVGMACAISILMKDLADEVALVDVMEDKLKGEMMDLQHGSLFLRTPKIVSGKDYNVTANSRLVIITAGARQQEGESRLNLVQRNVNIFKFIIPNIVKYSPNCKLLVVSNPVDILTYVAWKISGFPKNRVIGSGCNLDSARFRYLMGERLGVHPLSCHGWILGEHGDSSVPVWSGVNVAGVSLKNLHPELGTDADKEQWKAVHKQVVDSAYEVIKLKGYTSWAIGLSVADLAESIMKNLRRVHPISTMIKGLYGIKEDVFLSVPCILGQNGISDVVKVTLTHEEEACLKKSADTLWGIQKELQF

SEQ ID NO: 96 - DNA, 1575 bp, Artificial Sequence
  Synthesized: pct 7242549
atgagaaaggttcccattattaccgcagatgaggctgcaaagcttattaaagacggtgatacagttacaacaagtggtttcgttggaaatgcaatccctgaggctcttgatagagctgtagaaaaaagattcttagaaacaggcgaacccaaaaacattacatatgtttattgtggttctcaaggtaacagagacggaagaggtgctgagcactttgctcatgaaggccttttaaaacgttacatcgctggtcactgggctacagttcctgctttgggtaaaatggctatggaaaataaaatggaagcatataatgtatctcagggtgcattgtgtcatttgttccgtgatatagcttctcataagccaggcgtatttacaaaggtaggtatcggtactttcattgaccccagaaatggcggcggtaaagtaaatgatattaccaaagaagatattgttgaattggtagagattaagggtcaggaatatttattctaccctgcttttcctattcatgtagctcttattcgtggtacttacgctgatgaaagcggaaatatcacatttgagaaagaagttgctcctctggaaggaacttcagtatgccaggctgttaaaaacagtggcggtatcgttgtagttcaggttgaaagagtagtaaaagctggtactcttgaccctcgtcatgtaaaagttccaggaatttatgttgactatgttgttgttgctgacccagaagatcatcagcaatctttagattgtgaatatgatcctgcattatcaggcgagcatagaagacctgaagttgttggagaaccacttcctttgagtgcaaagaaagttattggtcgtcgtggtgccattgaattagaaaaagatgttgctgtaaatttaggtgttggtgcgcctgaatatgtagcaagtgttgctgatgaagaaggtatcgttgattttatgactttaactgctgaaagtggtgctattggtggtgttcctgctggtggcgttcgctttggtgcttcttataatgcggatgcattgatcgatcaaggttatcaattcgattactatgatggcggcggcttagacctttgctatttaggcttagctgaatgcgatgaaaaaggcaatatcaacgtttcaagatttggccctcgtatcgctggttgtggtggtttcatcaacattacacagaatacacctaaggtattcttctgtggtactttcacagcaggtggcttaaaggttaaaattgaagatggcaaggttattattgttcaagaaggcaagcagaaaaaattcttgaaagctgttgagcagattacattcaatggtgacgttgcacttgctaataagcaacaagtaacttatattacagaaagatgcgtattccttttgaaggaagatggtttgcacttatctgaaattgcacctggtattgatttgcagacacagattcttgacgttatggattttgcacctattattgacagagatgcaaacggccaaatcaaattgatggacgctgctttgtttgcagaaggcttaatgggtctgaaggaaatgaagtcctga

SEQ ID NO: 97 - protein, 524 aa, Artificial Sequence
  Synthesized: pct Protein Q9L3F7
MRKVPIITADEAAKLIKDGDTVTTSGFVGNAIPEALDRAVEKRFLETGEPKNITYVYCGSQGNRDGRGAEHFAHEGLLKRYIAGHWATVPALGKMAMENKMEAYNVSQGALCHLFRDIASHKPGVFTKVGIGTFIDPRNGGGKVNDITKEDIVELVEIKGQEYLFYPAFPIHVALIRGTYADESGNITFEKEVAPLEGTSVCQAVKNSGGIVVVQVERVVKAGTLDPRHVKVPGIYVDYVVVADPEDHQQSLDCEYDPALSGEHRRPEVVGEPLPLSAKKVIGRRGAIELEKDVAVNLGVGAPEYVASVADEEGIVDFMTLTAESGAIGGVPAGGVRFGASYNADALIDQGYQFDYYDGGGLDLCYLGLAECDEKGNINVSRFGPRIAGCGGFINITQNTPKVFFCGTFTAGGLKVKIEDGKVIIVQEGKQKKFLKAVEQITFNGDVALANKQQVTYITERCVFLLKEDGLHLSEIAPGIDLQTQILDVMDFAPIIDRDANGQIKLMDAALFAEGLMGLKEMKS

SEQ ID NO: 98 - DNA, 2142 bp, Artificial Sequence
  Synthesized: ACS1 296142482
atgtcgccctctgccgtacaatcatcaaaactagaagaacagtcaagtgaaattgacaagttgaaagcaaaaatgtcccagtctgccgccactgcgcagcagaagaaggaacatgagtatgaacatttgacttcggtcaagatcgtgccacaacggcccatctcagatagactgcagcccgcaattgctacccactattctccacacttggacgggttgcaggactatcagcgcttgcacaaggagtctattgaagaccctgctaagttcttcggttctaaagctacccaatttttaaactggtctaagccattcgataaggtgttcatcccagaccctaaaacgggcaggccctccttccagaacaatgcatggttcctcaacggccaattaaacgcctgttacaactgtgttgacagacatgccttgaagactcctaacaagaaagccattattttcgaaggtgacgagcctggccaaggctattccattacctacaaggaactacttgaagaagtttgtcaagtggcacaagtgctgacttactctatgggcgttcgcaagggcgatactgttgccgtgtacatgcctatggtcccagaagcaatcataaccttgttggccatttcccgtatcggtgccattcactccgtagtctttgccgggttttcttccaactccttgagagatcgtatcaacgatggggactctaaagttgtcatcactacagatgaatccaacagaggtggtaaagtcattgagactaaaagaattgttgatgacgcgctaagagagaccccaggcgtgagacacgtcttggtttatagaaagaccaacaatccatctgttgctttccatgcccccagagatttggattgggcaacagaaaagaagaaatacaagacctactatccatgcacacccgttgattctgaggatccattattcttgttgtatacgtctggttctactggtgcccccaagggtgttcaacattctaccgcaggttacttgctgggagctttgttgaccatgcgctacacttttgacactcaccaagaagacgttttcttcacagctggagacattggctggattacaggccacacttatgtggtttatggtcccttactatatggttgtgccactttggtctttgaagggactcctgcgtacccaaattactcccgttattgggatattattgatgaacacaaagtcacccaattttatgttgcgccaactgctttgcgtttgttgaaaagagctggtgattcctacatcgaaaatcattccttaaaatctttgcgttgcttgggttcggtcggtgagccaattgctgctgaagtttgggagtggtactctgaaaaaataggtaaaaatgaaatccccattgtagacacctactggcaaacagaatctggttcgcatctggtcaccccgctggctggtggtgttacaccaatgaaaccgggttctgcctcattccccttcttcggtattgatgcagttgttcttgaccctaacactggtgaagaacttaacaccagccacgcagagggtgtccttgccgtcaaagctgcatggccatcatttgcaagaactatttggaaaaatcatgataggtatctagacacttatttgaacccttaccctggctactatttcactggtgatggtgctgcaaaggataaggatggttatatctggattttgggtcgtgtagacgatgtggtgaacgtctctggtcaccgtctgtctaccgctgaaattgaggctgctattatcgaagatccaattgtggccgagtgtgctgttgtcggattcaacgatgacttgactggtcaagcagttgctgcatttgtggtgttgaaaaacaaatctagttggtccaccgcaacagatgatgaattacaagatatcaagaagcatttggtctttactgttagaaaagacatcgggccatttgccgcaccaaaattgatcattttagtggatgacttgcccaagacaagatccggcaaaattatgagacgtattttaagaaaaatcctagcaggagaaagtgaccaactaggcgacgtttctacattgtcaaaccctggcattgttagacatctaattgattcggtcaagttgtaa

SEQ ID NO: 99 - protein, 713 aa, Artificial Sequence
  Synthesized: ACS1 Protein Q01574
MSPSAVQSSKLEEQSSEIDKLKAKMSQSAATAQQKKEHEYEHLTSVKIVPQRPISDRLQPAIATHYSPHLDGLQDYQRLHKESIEDPAKFFGSKATQFLNWSKPFDKVFIPDPKTGRPSFQNNAWFLNGQLNACYNCVDRHALKTPNKKAIIFEGDEPGQGYSITYKELLEEVCQVAQVLTYSMGVRKGDTVAVYMPMVPEAIITLLAISRIGAIHSVVFAGFSSNSLRDRINDGDSKVVITTDESNRGGKVIETKRIVDDALRETPGVRHVLVYRKTNNPSVAFHAPRDLDWATEKKKYKTYYPCTPVDSEDPLFLLYTSGSTGAPKGVQHSTAGYLLGALLTMRYTFDTHQEDVFFTAGDIGWITGHTYVVYGPLLYGCATLVFEGTPAYPNYSRYWDIIDEHKVTQFYVAPTALRLLKRAGDSYIENHSLKSLRCLGSVGEPIAAEVWEWYSEKIGKNEIPIVDTYWQTESGSHLVTPLAGGVTPMKPGSASFPFFGIDAVVLDPNTGEELNTSHAEGVLAVKAAWPSFARTIWKNHDRYLDTYLNPYPGYYFTGDGAAKDKDGYIWILGRVDDVVNVSGHRLSTAEIEAAIIEDPIVAECAVVGFNDDLTGQAVAAFVVLKNKSSWSTATDDELQDIKKHLVFTVRKDIGPFAAPKLIILVDDLPKTRSGKIMRRILRKILAGESDQLGDVSTLSNPGIVRHLIDSVKL

SEQ ID NO: 100 - DNA, 1395 bp, Artificial Sequence
  Synthesized: pduP 1253572
atgaatacttctgaactcgaaaccctgattcgcaccattcttagcgagcaattaaccacgccggcgcaaacgccggtccagcctcagggcaaagggattttccagtccgtgagcgaggccatcgacgccgcgcaccaggcgttcttacgttatcagcagtgcccgctaaaaacccgcagcgccattatcagcgcgatgcgtcaggagctgacgccgctgctggcgcccctggcggaagagagcgccaatgaaacggggatgggcaacaaagaagataaatttctcaaaaacaaggctgcgctggacaacacgccgggcgtagaagatctcaccaccaccgcgctgaccggcgacggcggcatggtgctgtttgaatactcaccgtttggcgttatcggttcggtcgccccaagcaccaacccgacggaaaccatcatcaacaacagtatcagcatgctggcggcgggcaacagtatctactttagcccgcatccgggagcgaaaaaggtctctctgaagctgattagcctgattgaagagattgccttccgctgctgcggcatccgcaatctggtggtgaccgtggcggaacccaccttcgaagcgacccagcagatgatggcccacccgcgaatcgcagtactggccattaccggcggcccgggcattgtggcaatgggcatgaagagcggtaagaaggtgattggcgctggcgcgggtaacccgccctgcatcgttgatgaaacggcggacctggtgaaagcggcggaagatatcatcaacggcgcgtcattcgattacaacctgccctgcattgccgagaagagcctgatcgtagtggagagtgtcgccgaacgtctggtgcagcaaatgcaaaccttcggcgcgctgctgttaagccctgccgataccgacaaactccgcgccgtctgcctgcctgaaggccaggcgaataaaaaactggtcggcaagagcccatcggccatgctggaagccgccgggatcgctgtccctgcaaaagcgccgcgtctgctgattgcgctggttaacgctgacgatccgtgggtcaccagcgaacagttgatgccgatgctgccagtggtaaaagtcagcgatttcgatagcgcgctggcgctggccctgaaggttgaagaggggctgcatcataccgccattatgcactcgcagaacgtgtcacgcctgaacctcgcggcccgcacgctgcaaacctcgatattcgtcaaaaacggcccctcttatgccgggatcggcgtcggcggcgaaggctttaccaccttcactatcgccacaccaaccggtgaagggaccacgtcagcgcgtacttttgcccgttcccggcgctgcgtactgaccaacggcttttctattcgctaa

SEQ ID NO: 101 - protein, 464 aa, Artificial Sequence
  Synthesized: pduP Protein Q9XDN1
MNTSELETLIRTILSEQLTTPAQTPVQPQGKGIFQSVSEAIDAAHQAFLRYQQCPLKTRSAIISAMRQELTPLLAPLAEESANETGMGNKEDKFLKNKAALDNTPGVEDLTTTALTGDGGMVLFEYSPFGVIGSVAPSTNPTETIINNSISMLAAGNSIYFSPHPGAKKVSLKLISLIEEIAFRCCGIRNLVVTVAEPTFEATQQMMAHPRIAVLAITGGPGIVAMGMKSGKKVIGAGAGNPPCIVDETADLVKAAEDIINGASFDYNLPCIAEKSLIVVESVAERLVQQMQTFGALLLSPADTDKLRAVCLPEGQANKKLVGKSPSAMLEAAGIAVPAKAPRLLIALVNADDPWVTSEQLMPMLPVVKVSDFDSALALALKVEEGLHHTAIMHSQNVSRLNLAARTLQTSIFVKNGPSYAGIGVGGEGFTTFTIATPTGEGTTSARTFARSRRCVLTNGFSIR

SEQ ID NO: 102 - DNA, 1149 bp, Artificial Sequence; identical to SEQ ID NO: 76
  Synthesized: fucO 947273

SEQ ID NO: 103 - protein, 382 aa, Artificial Sequence; identical to SEQ ID NO: 77
  Synthesized: fucO Protein P0A9S1

SEQ ID NO: 104 - DNA, 1401 bp, Artificial Sequence
  Synthesized: udhA 948461
atgccacattcctacgattacgatgccatagtaataggttccggccccggcggcgaaggcgctgcaatgggcctggttaagcaaggtgcgcgcgtcgcagttatcgagcgttatcaaaatgttggcggcggttgcacccactggggcaccatcccgtcgaaagctctccgtcacgccgtcagccgcattatagaattcaatcaaaacccactttacagcgaccattcccgactgctccgctcttcttttgccgatatccttaaccatgccgataacgtgattaatcaacaaacgcgcatgcgtcagggattttacgaacgtaatcactgtgaaatattgcagggaaacgctcgctttgttgacgagcatacgttggcgctggattgcccggacggcagcgttgaaacactaaccgctgaaaaatttgttattgcctgcggctctcgtccatatcatccaacagatgttgatttcacccatccacgcatttacgacagcgactcaattctcagcatgcaccacgaaccgcgccatgtacttatctatggtgctggagtgatcggctgtgaatatgcgtcgatcttccgcggtatggatgtaaaagtggatctgatcaacacccgcgatcgcctgctggcatttctcgatcaagagatgtcagattctctctcctatcacttctggaacagtggcgtagtgattcgtcacaacgaagagtacgagaagatcgaaggctgtgacgatggtgtgatcatgcatctgaagtcgggtaaaaaactgaaagctgactgcctgctctatgccaacggtcgcaccggtaataccgattcgctggcgttacagaacattgggctagaaactgacagccgcggacagctgaaggtcaacagcatgtatcagaccgcacagccacacgtttacgcggtgggcgacgtgattggttatccgagcctggcgtcggcggcctatgaccaggggcgcattgccgcgcaggcgctggtaaaaggcgaagccaccgcacatctgattgaagatatccctaccggtatttacaccatcccggaaatcagctctgtgggcaaaaccgaacagcagctgaccgcaatgaaagtgccatatgaagtgggccgcgcccagtttaaacatctggcacgcgcacaaatcgtcggcatgaacgtgggcacgctgaaaattttgttccatcgggaaacaaaagagattctgggtattcactgctttggcgagcgcgctgccgaaattattcatatcggtcaggcgattatggaacagaaaggtggcggcaacactattgagtacttcgtcaacaccacctttaactacccgacgatggcggaagcctatcgggtagctgcgttaaacggtttaaaccgcctgttttaa

SEQ ID NO: 105 - protein, 466 aa, Artificial Sequence
  Synthesized: udhA Protein P27306
MPHSYDYDAIVIGSGPGGEGAAMGLVKQGARVAVIERYQNVGGGCTHWGTIPSKALRHAVSRIIEFNQNPLYSDHSRLLRSSFADILNHADNVINQQTRMRQGFYERNHCEILQGNARFVDEHTLALDCPDGSVETLTAEKFVIACGSRPYHPTDVDFTHPRIYDSDSILSMHHEPRHVLIYGAGVIGCEYASIFRGMDVKVDLINTRDRLLAFLDQEMSDSLSYHFWNSGVVIRHNEEYEKIEGCDDGVIMHLKSGKKLKADCLLYANGRTGNTDSLALQNIGLETDSRGQLKVNSMYQTAQPHVYAVGDVIGYPSLASAAYDQGRIAAQALVKGEATAHLIEDIPTGIYTIPEISSVGKTEQQLTAMKVPYEVGRAQFKHLARAQIVGMNVGTLKILFHRETKEILGIHCFGERAAEIIHIGQAIMEQKGGGNTIEYFVNTTFNYPTMAEAYRVAALNGLNRLF

SEQ ID NO: 106 - DNA, 1017 bp, Artificial Sequence
  Synthesized: mdh 28865823
atggaagcacttgtgttaactggtacaaaaaaattagaggttgaaaacattgaacaacctgaggtaaagccgaatgaagtgttgattcatacagcattcgctggtatttgcggtactgatcacgctttgtatgccggtcttcctggctcagccgatgctgtgccaccaatcgttttggggcatgaaaattctggtgttgtagctgaaattggttctgatgttacaaacgttgcggtgggtgatcgtgtcacaattgatcccaatatttactgtggtcaatgcaagtattgccgtacagcacgtccagagctttgcgaaaacttgtctgcagttggtgtaacacgcaatggtggctttgaagaatactttactgcgcccgcatcagttgtttaccaaattccagataatgtttcacttaagtcagctgccgtggttgagccgatttcatgtgctgttcacggtattcaacttcttaaagtgacaccataccaaaaggcattagttattggtgacggcttcatgggtgaactctttgttcaaattctgcaagcttatggcattcaccaagtcgacttggctggtattgttcctgaaaagcttgctatgaacaaagaaaagttcggcgtgaaaaatacgtacaatacaaaagatggcgacaaaattcccgaaggcacttacgatgttgttgttgaagcagttggcctaccacagacacaagaagccgcaattgaagcctcagctcgtggcgctcaggttttgatgtttggtgttggcggtcccgacgcaaagttccaaatgaacacttacgaagtcttccaaaagcaattgacgattcaaggatcatttatcaatccaaacgcatttgaagactcattggcattgttatcatcaggcaagttagacgtcgaatcgctaatgtcacacgaattagattaccagactgttgatgactttgtgaatggcaagttaggtgtcgtttcaaaggcagtcgttaaggttggtggcgaagaggcataa

SEQ ID NO: 107 - protein, 338 aa, Artificial Sequence
  Synthesized: mdh Protein Q83V15
MEALVLTGTKKLEVENIEQPEVKPNEVLIHTAFAGICGTDHALYAGLPGSADAVPPIVLGHENSGVVAEIGSDVTNVAVGDRVTIDPNIYCGQCKYCRTARPELCENLSAVGVTRNGGFEEYFTAPASVVYQIPDNVSLKSAAVVEPISCAVHGIQLLKVTPYQKALVIGDGFMGELFVQILQAYGIHQVDLAGIVPEKLAMNKEKFGVKNTYNTKDGDKIPEGTYDVVVEAVGLPQTQEAAIEASARGAQVLMFGVGGPDAKFQMNTYEVFQKQLTIQGSFINPNAFEDSLALLSSGKLDVESLMSHELDYQTVDDFVNGKLGVVSKAVVKVGGEEA

SEQ ID NO: 108 - DNA, 63 bp, Artificial Sequence
  Synthesized: BKO592
actcataacctcacgcaaaataacacagtcaaatcaatcaaaagcttttcaattcaattcatc

SEQ ID NO: 109 - DNA, 61 bp, Artificial Sequence
  Synthesized: BKO593
aatgcttataaaactttaactaataattagagattaaatcgcgggtaataactgatataat

SEQ ID NO: 110 - DNA, 200 bp, Artificial Sequence
  Synthesized: BKO678
cctagatcgatttgattacaggataagggatatggtgcgattcgcgagcttttaccaatatgtataaaaggcggctgtttgaagccattctatcttaatcttgtgctattgcagtcctcttttatatacagtataaataaaaaacccacgtaatatagcaaaaacatattgccaacaaaagcttttcaattcaattcatc

SEQ ID NO: 111 - DNA, 199 bp, Artificial Sequence
  Synthesized: BKO679
gggtaataactgatataatgccattagtagtgtactcaaacgaattattgttgcaaataaataaacttacacagtttgaatacataaatcaatcagacaaataaatacatcggttcaaattatactaaatctaaatactacgttatcgccgtgaattacgcaattcgcatgttacgtactgcgcgtctcttgttgaata

SEQ ID NO: 112 - DNA, 198 bp, Artificial Sequence
  Synthesized: BKO909
tcaagttcctcgatactagttatttgtaatacgtatacgaattccttcaacaaaggccaaggaaataaagcaaataacaataacaccattattttaattttttttctattactgtcgctaacacctgtatggttgcaaccaggtgagaatccttctgatgcatactttatgcgtttatgctacgctgcaggtcgacaa

SEQ ID NO: 113 - DNA, 200 bp, Artificial Sequence
  Synthesized: BKO910
gctaaaggtacaaaaccgaatacgaaagtaaataaattaatcagcataaaattaaataataaaccacctaaaatattagaagctaatctttaacctggaagacaggacagaaaagtaattacaagaacatatgtgaaaaaaaatagttgatattttaaaccaaatcagaaatttattatacagagcgcccaatacgcaaa

(19) United States
(12) Patent Application Publication
(10) Pub. No.: US 2015/0064759 A1
(43) Pub. Date: Mar. 5, 2015

(54) MODIFIED MICROORGANISM AND METHODS OF USING SAME FOR PRODUCING 2-PROPANOL AND1-PROPANOL AND/OR 1,2-PROPANEDIOL

(71) Applicant: Braskem S.A., Camacari (BR)

(72) Inventors: Johana Rincones Perez, Camacari (BR); Juan Diego Rojas Rojas, Camacari (BR); Ane Fernanda Beraldi Zeidler, Camacari (BR); Aline Silva Romao Dumaresq, Camacari (BR); Marilene Elizabete Pavan Rodrigues, Camacari (BR); Iuri Estrada Gouvea, Camacari (BR); Felipe Galzerani, Camacari (BR); Daniel Johannes Koch, Camacari (BR); Lucas Pedersen Parizzi, Camacari (BR); Mateus Schreiner Garcez Lopes, Camacari (BR); Thomas Martin Halder, Camacari (BR); Antonio Luis Ribeiro De Castro Morschbacker, Camacari (BR); Avram Michael Slovic, Camacari (BR)

(21) Appl. No.: 14/478,737

(22) Filed: Sep. 5, 2014

Related U.S. Application Data
(60) Provisional application No. 61/874,200, filed on Sep. 5, 2013.

(51) Int. Cl. C12P 7/18; C12P 7/04

(52) U.S. Cl. CPC C12P 7/18; C12P 7/04
USPC 435/158; 435/252.3; 435/157; 435/252.31; 435/252.33; 435/254.2; 435/254.11; 435/257.2; 435/258.1; 435/254.21; 435/254.23

(57) ABSTRACT

The present disclosure provides a non-naturally occurring microorganism comprising: one or more polynucleotides encoding one or more enzymes in a pathway that produces acetyl-CoA; one or more polynucleotides encoding one or more enzymes in a pathway that catalyze a conversion of cytosolic acetyl-CoA to 2-propanol; one or more polynucleotides encoding one or more enzymes in a pathway that catalyze a conversion of dihydroxyacetone-phosphate to 1-propanol and/or 1,2-propanediol, wherein the microorganism has reduced levels of pyruvate decarboxylase enzymatic activity (e.g., the microorganism comprises a disruption of one or more enzymes that decarboxylate pyruvate and/or a disruption of one or more transcription factors of one or more enzymes that decarboxylate pyruvate), and wherein the microorganism is capable of growing on a C6 sugar as a sole carbon source under anaerobic conditions. Also provided are methods of using the disclosed non-naturally occurring microorganisms in methods for the coproduction of 2-propanol and 1-propanol and/or 1,2-propanediol.

### BACKGROUND

1-propanol (n-propanol, CH₃CH₂CH₂OH, CAS 71-23-8) is a primary alcohol typically manufactured by catalytic hydrogenation of propionaldehyde, which is generally synthesized in large scale from ethylene in an energy-intensive multi-step industrial process. This process involves use of toxic chemicals such as carbon monoxide and hydrogen at high pressure (e.g., 10-100 ATM) and high temperature (up to 200° C.). 1-propanol can be used as an intermediate for further organic reactions or as a building block for polymers such as propylene. Propylene is a chemical compound that is widely used to synthesize a wide range of petrochemical products. For instance, this olefin is the raw material used for the production of polypropylene, its copolymers and other chemicals such as acrylonitrile, acrylic acid, epichloridrine and acetone. Propylene is typically obtained in large quantity scales as a byproduct of catalytical or thermal oil cracking, or as a co-product of ethylene production from natural gas. (Propylene, Jamie G. Lacson, CEH Marketing Research Report-2004, Chemical Economics Handbook-SRI International). Propylene is polymerized to produce thermoplastics resins for innumerous applications such as rigid or flexible packaging materials, blow molding and injection molding.

2-propanol (isopropyl alcohol, CH₃CH₃CHOH, CAS 67-63-0) is a secondary alcohol and is a structural isomer of 1-propanol. 2-propanol is typically produced by the weak acid process in which propene gas is absorbed in, and reacted with, 60% sulfuric acid and the resulting sulfates hydrolyzed in a single step process. Another major current manufacturing process is catalytic hydration of propylene with water. Hydration can be gas phase with a phosphoric acid catalyst, mixed phase with a cation-exchange resin catalyst or liquid phase using a tungsten catalyst. 2-propanol is used as an industrial solvent, a component of industrial and consumer products and as a disinfectant. Most 2-propanol goes into the solvent market either directly or via conversion to acetone or one of acetone's derivatives—methyl isobutyl ketone, methyl isobutyl carbinol, diacetone alcohol, or isophorone. 2-propanol's major solvent uses include inks, coatings, cosmetics and pharmaceuticals.

1,2-propanediol (propylene glycol, HO—CH₂—CHOH—CH₃, CAS 57-55-6) is an organic compound with formula C₃H₈O₂. Industrially, propylene glycol is produced from propylene oxide. Propylene glycol may be manufactured using either a non-catalytic high-temperature process at 200° C. (392° F.) to 220° C. (428 F), or a catalytic method, which proceeds at 150° C. (302° F.) to 180° C. (356° F.) in the presencefdon exchange resin or a small amount of sulfuric acid or alkali. Propylene glycol can be used as a solvent, nontoxic antifreeze and to produce polyesteres compounds.

Given the world-wide demand for 2-propanol, 1-propanol, and 1,2-propanediol, there exits a need in the art for improved methods for their production that overcome their current production drawbacks including the use of toxic and/or expensive catalysts, and highly flammable and/or gaseous carbon sources.

### SUMMARY

The present disclosure provides a non-naturally occurring microorganism comprising: one or more exogenous polynucleotides encoding one or more enzymes in a pathway that produces cytosolic acetyl-CoA (i.e., acetyl-CoA is produced in the cytosol of the microorganism), wherein the microorganism has reduced levels of pyruvate decarboxylase enzymatic activity, and wherein the microorganism is capable of growing on a C6 sugar as a sole carbon source and under anaerobic conditions.

The present disclosure covers the co-production of 1,2-propanol or 1-propanol and 2-propanol in a eukaryote cell, such as a yeast, with reduced levels of pyruvate decarboxylase enzymatic activity, wherein the microorganism has its native ethanol production shut-off, and wherein the microorganism is capable of growing on a C6 sugar as a sole carbon source under anaerobic or microaerobic conditions.

In order to eliminate the ethanol production in yeast it is necessary to knock out the activity of pyruvate decarboxylase, the enzyme that decarboxylates pyruvate making acetaldehyde and carbon dioxide. In yeast, this enzyme comes in three isoforms, and its activity can be completely knocked out by deleting the genes PDC1, PDC5 and PDC6. As a consequence, the microorganism can not grow on C6 sugars as a sole carbon source such as glucose and consequently it is necessary to alter the ability of the microorganism to import glucose, for example, by truncating a transcription factor of the glucose importer MTH1. Also, the elimination of the pyruvate decarboxylase activity in the cell's cytoplasm renders the microorganism unable to grow under anaerobic conditions due to two factors: (1) the lack of an alternative route for cytoplasmic acetyl-CoA production, due to the lack of acetaldehyde that would be converted to acetate and acetyl-CoA; and (2) a redox imbalance due to excess NADH because the NADH is no longer oxidized in the conversion of acetaldehyde to ethanol. Accordingly, the present disclosure discloses a series of complex deletions/truncations and gene integrations that enables a new acetil-CoA overproducing yeast chassis for the co-production of 1,2-propanol or 1-propanol and 2-propanol.

The present disclosure also provides a non-naturally occurring microorganism comprising: a disruption of one or more enzymes that decarboxylate pyruvate and/or a disruption of one or more transcription factors of one or more enzymes that decarboxylate pyruvate; a genetic modification that substantially decreases glucose import into the microorganism; one or more polynucleotides encoding one or more enzymes in a pathway that produces cytosolic acetyl-CoA; one or more polynucleotides encoding one or more enzymes in a pathway that catalyze a conversion of cytosolic acetyl-CoA to 2-propanol; and one or more polynucleotides encoding one or more enzymes in a pathway that catalyze a conversion of dihydroxyacetone-phosphate to 1-propanol and/or 1,2-propanediol.

In some embodiments of each or any of the above or below mentioned embodiments, the disruption in the one or more enzymes that decarboxylate pyruvate is a deletion or a mutation.

In some embodiments of each or any of the above or below mentioned embodiments, the one or more enzymes that decarboxylate pyruvate include pdc1, pdc 5, and/or pdc6, and wherein the one or more transcription factors of the one or more enzymes that decarboxylate pyruvate include pdc2.

In some embodiments of each or any of the above or below mentioned embodiments, the microorganism comprises an exogenous polynucleotide that encodes a transcription factor involved in glucose import.

In some embodiments of each or any of the above or below mentioned embodiments, the microorganism comprises a genetic modification in an endogenous polynucleotide that encodes a transcription factor involved in glucose import.

In some embodiments of each or any of the above or below mentioned embodiments, the genetic modification is a truncation of the MTH1 transcription factor.

In some embodiments of each or any of the above or below mentioned embodiments, the one or more exogenous polynucleotides encoding one or more enzymes in a pathway that produces acetyl-CoA encode i.) pyruvate formate lyase and pyruvate formate lyase activating enzyme, ii) pyruvate dehydrogenase, dihydrolipoyl transacetylase and dihydrolipoamide dehydrogenase, iii) pyruvate dehydrogenase, dihydrolipoyl transacetylase, dihydrolipoamide dehydrogenase, and pyruvate dehydrogenase complex protein X, or any combination thereof.

In some embodiments of each or any of the above or below mentioned embodiments, the microorganism is a eukaryote selected from the group consisting of: yeast, filamentous fungi, protozoa, and algae.

In some embodiments of each or any of the above or below mentioned embodiments, the one or more polynucleotides coding for enzymes in a pathway that catalyzes a conversion of acetyl-CoA to 2-propanol include: one or more polynucleotides coding for enzymes in a pathway that catalyze a conversion of acetyl-CoA to acetoacetyl-CoA, one or more polynucleotides coding for enzymes in a pathway that catalyze a conversion of acetoacetyl-CoA to acetoacetate, one or more polynucleotides coding for enzymes in a pathway that catalyze a conversion of acetoacetate to acetone, and/or one or more polynucleotides coding for enzymes in a pathway that catalyze a conversion of acetone to 2-propanol.

In some embodiments of each or any of the above or below mentioned embodiments, the one or more polynucleotides coding for enzymes in a pathway that catalyzes a conversion of dihydroxyacetone-phosphate to 1-propanol and/or 1,2-propanediol include: one or more polynucleotides coding for enzymes in a pathway that catalyze a conversion of dihydroxyacetone-phosphate to methylglyoxal, one or more polynucleotides coding for enzymes in a pathway that catalyze a conversion of methylglyoxal to hydroxyacetone, one or more polynucleotides coding for enzymes in a pathway that catalyze a conversion of hydroxyacetone to 1,2-propanediol, one or more polynucleotides coding for enzymes in a pathway that catalyze a conversion of methylglyoxal to lactaldehyde, one or more polynucleotides coding for enzymes in a pathway that catalyze a conversion of lactaldehyde to 1,2-propanediol, one or more polynucleotides coding for enzymes in a pathway that catalyze a conversion of 1,2-propanediol to propionaldehyde, and/or one or more polynucleotides coding for enzymes in a pathway that catalyze a conversion of propionaldehyde to 1-propanol.

The present disclosure also provides a non-naturally occurring microorganism comprising: one or more exogenous polynucleotides encoding one or more enzymes in a pathway that produces cytosolic acetyl-CoA; one or more polynucleotides coding for enzymes that catalyze a conversion of cytosolic acetyl-CoA to 2-propanol; and one or more polynucleotides coding for enzymes in a pathway that catalyze a conversion of dihydroxyacetone-phosphate to 1-propanol and/or 1,2-propanediol, wherein the microorganism has reduced levels of pyruvate decarboxylase enzymatic activity, and wherein the microorganism is capable of growing on a C6 sugar as a sole carbon source under anaerobic conditions.

In some embodiments of each or any of the above or below mentioned embodiments, the microorganism has a disruption in one or more polynucleotides that code for one or more enzymes that decarboxylate pyruvate or a disruption in one or more polynucleotides that code for a transcription factor of an enzyme that decarboxylates pyruvate.

In some embodiments of each or any of the above or below mentioned embodiments, the disruption in the one or more enzymes that decarboxylate pyruvate is a deletion or a mutation.

In some embodiments of each or any of the above or below mentioned embodiments, the one or more enzymes that decarboxylate pyruvate include pdc1, pdc 5, and/or pdc6, and wherein the one or more transcription factors of the one or more enzymes that decarboxylate pyruvate include pdc2.

In some embodiments of each or any of the above or below mentioned embodiments, the one or more polynucleotides coding for enzymes in a pathway that catalyzes a conversion of acetyl-CoA to 2-propanol include: one or more polynucleotides coding for enzymes in a pathway that catalyze a conversion of acetyl-CoA to acetoacetyl-CoA, one or more polynucleotides coding for enzymes in a pathway that catalyze a conversion of acetoacetyl-CoA to acetoacetate, one or more polynucleotides coding for enzymes in a pathway that catalyze a conversion of acetoacetate to acetone, and/or one or more polynucleotides coding for enzymes in a pathway that catalyze a conversion of acetone to 2-propanol.

In some embodiments of each or any of the above or below mentioned embodiments, the one or more polynucleotides coding for enzymes in a pathway that catalyzes a conversion of dihydroxyacetone-phosphate to 1-propanol include and/or 1,2-propanediol: one or more polynucleotides coding for enzymes in a pathway that catalyze a conversion of dihydroxyacetone-phosphate to methylglyoxal, one or more polynucleotides coding for enzymes in a pathway that catalyze a conversion of methylglyoxal to hydroxyacetone, one or more polynucleotides coding for enzymes in a pathway that catalyze a conversion of hydroxyacetone to 1,2-propanediol, one or more polynucleotides coding for enzymes in a pathway that catalyze a conversion of methylglyoxal to lactaldehyde, one or more polynucleotides coding for enzymes in a pathway that catalyze a conversion of lactaldehyde to 1,2-propanediol, one or more polynucleotides coding for enzymes in a pathway that catalyze a conversion of 1,2-propanediol to propionaldehyde, and/or one or more polynucleotides coding for enzymes in a pathway that catalyze a conversion of propionaldehyde to 1-propanol.

The present disclsoure also provides methods for co-producing 2-propanol with 1-propanol and/or 1,2-propanediol from a fermentable carbon source under anaerobic conditions, the method comprising: providing a fermentable carbon source; contacting the fermentable carbon source with the non-naturally occurring microorganism as disclosed herein in a fermentation media under substantially anaerobic conditions, and expressing the polynucleotides in the microorganism for the co-production of 2-propanol with 1-propanol and/or 1,2-propanediol, wherein the microorganism co-produces 2-propanol with 1-propanol and/or 1,2-propanediol.

In some embodiments of each or any of the above or below mentioned embodiments, the fermentable carbon source is sugarcane juice, sugarcane molasses, hydrolyzed starch, hydrolyzed lignocellulosic materials, glucose, sucrose, fructose, lactate, lactose, xylose, pyruvate, or glycerol in any form or mixture thereof.

In some embodiments of each or any of the above or below mentioned embodiments, the fermentable carbon source is a monosaccharide, oligosaccharide, or polysaccharide.

In some embodiments of each or any of the above or below mentioned embodiments, the produced 2-propanol with 1-propanol and/or 1,2-propanediol are secreted by the microorganism into the fermentation media.

In some embodiments of each or any of the above or below mentioned embodiments, the methods further comprise recovering the produced 2-propanol with 1-propanol and/or 1,2-propanediol from the fermentation media.

In some embodiments of each or any of the above or below mentioned embodiments, the microorganism has no detectable pyruvate decarboxylase enzymatic activity.

In some embodiments of each or any of the above or below mentioned embodiments, the microorganism has a disruption in one or more polynucleotides that code for one or more enzymes that decarboxylate pyruvate (e.g., a pyruvate decarboxylase) or a disruption in one or more polynucleotides that code for a transcription factor of an enzyme that decarboxylates pyruvate.

In some embodiments of each or any of the above or below mentioned embodiments, the microorganism has a disruption in each of the one or more polynucleotides that code for enzymes that decarboxylate pyruvate or a disruption in each of the polynucleotides that code for a transcription factor of an enzyme that decarboxylates pyruvate.

In some embodiments of each or any of the above or below mentioned embodiments, the disruption in the one or more polynucleotides is a deletion or a mutation.

In some embodiments of each or any of the above or below mentioned embodiments, the one or more polynucleotides that code for enzymes that decarboxylate pyruvate code for pdc1, pdc5, and/or pdc6. In some embodiments of each or any of the above or below mentioned embodiments, the one or more polynucleotides that code for a transcription factor of one or more enzymes that decarboxylates pyruvate code for pdc2.

In some embodiments of each or any of the above or below mentioned embodiments, the microorganism comprises an exogenous polynucleotide that encodes a transcription factor involved in glucose import.

In some embodiments of each or any of the above or below mentioned embodiments, the microorganism comprises a genetic modification in an endogenous polynucleotide that encodes a transcription factor involved in glucose import.

In some embodiments of each or any of the above or below mentioned embodiments, the genetic modification is a truncation of the MTH1 transcription factor. In an embodiment, the MTH1 transcription factor may have the amino acid sequence as set forth in SEQ ID NO: 1 and the truncated MTH1 transcription factor may have the amino acid sequence set forth in SEQ ID NO: 2.

In some embodiments of each or any of the above or below mentioned embodiments, the truncated MTH1 transcription factor has a longer half-life than an untruncated MTH1 transcription factor.

In some embodiments of each or any of the above or below mentioned embodiments, the one or more exogenous polynucleotides encoding one or more enzymes in a pathway that produces cytosolic acetyl-CoA encode i.) pyruvate formate lyase and pyruvate formate lyase activating enzyme, ii) pyruvate dehydrogenase, dihydrolipoyl transacetylase and dihydrolipoamide dehydrogenase, iii) pyruvate dehydrogenase, dihydrolipoyl transacetylase, dihydrolipoamide dehydrogenase, and pyruvate dehydrogenase complex protein X, or any combination thereof.

In some embodiments of each or any of the above or below mentioned embodiments, the microorganism is a eukaryote.

In some embodiments of each or any of the above or below mentioned embodiments, the eukaryote is a yeast, filamentous fungi, protozoa, or algae.

In some embodiments of each or any of the above or below mentioned embodiments, the microorganism further comprises one or more polynucleotides coding for an acetoacetyl-CoA hydrolase.

In some embodiments of each or any of the above or below mentioned embodiments, the acetoacetyl-CoA hydrolase is produced by introducing a mutation into the polynucleotide that encodes acetoacetyl-CoA:acetate transferase. In some embodiments of each or any of the above or below mentioned embodiments, the mutation is a E51D Glu-Asp mutation corresponding to the numbering of SEQ ID NO: 3.

In some embodiments of each or any of the above or below mentioned embodiments, the microorganism comprises one or more exogenous polynucleotides encoding one or more enzymes in pathways for the co-production of 1,2-propanediol and 2-propanol from a fermentable carbon source under anaerobic conditions.

In some embodiments of each or any of the above or below mentioned embodiments, the microorganism further comprises one or more polynucleotides coding for enzymes in a pathway that catalyzes a conversion of 1,2-propanediol to propanaldehyde.

In some embodiments of each or any of the above or below mentioned embodiments, the enzyme is a B12-independent dehydratase.

In some embodiments of each or any of the above or below mentioned embodiments, the B12-independent dehydratase is from Clostridium acetobutylicum, Clostridium glycolicum, Clostridium butyricum or Roseburia inulinivorans.

In some embodiments of each or any of the above or below mentioned embodiments, the microorganism comprises one or more exogenous polynucleotides encoding one or more enzymes in pathways for the co-production of 2-propanol, 1, propanol and/or 1,2-propanediol from a fermentable carbon source under anaerobic conditions.

In some embodiments of each or any of the above or below mentioned embodiments, the microorganism comprises one or more polynucleotides coding for enzymes in a pathway that catalyzes a conversion of acetyl-CoA to 2-propanol.

In some embodiments of each or any of the above or below mentioned embodiments, the one or more polynucleotides coding for enzymes in a pathway that catalyzes a conversion of acetyl-CoA to 2-propanol include: one or more polynucleotides coding for enzymes in a pathway that catalyze a conversion of acetyl-CoA to acetoacetyl-CoA, one or more polynucleotides coding for enzymes in a pathway that catalyze a conversion of acetoacetyl-CoA to acetoacetate, one or more polynucleotides coding for enzymes in a pathway that catalyze a conversion of acetoacetate to acetone, and/or one or more polynucleotides coding for enzymes in a pathway that catalyze a conversion of acetone to 2-propanol.

In some embodiments of each or any of the above or below mentioned embodiments, the microorganism comprises one or more polynucleotides coding for enzymes in a pathway that catalyzes a conversion of dihydroxyacetone-phosphate to 1,2-propanediol

In some embodiments of each or any of the above or below mentioned embodiments, the one or more polynucleotides coding for enzymes in a pathway that catalyzes a conversion of dihydroxyacetone-phosphate to 1,2-propanediol include: one or more polynucleotides coding for enzymes in a pathway that catalyze a conversion of dihydroxyacetone-phosphate to methylglyoxal, one or more polynucleotides coding for enzymes in a pathway that catalyze a conversion of methylglyoxal to hydroxyacetone, one or more polynucleotides coding for enzymes in a pathway that catalyze a conversion of hydroxyacetone to 1,2-propanediol, one or more polynucleotides coding for enzymes in a pathway that catalyze a conversion of methylglyoxal to lactaldehyde and/or one or more polynucleotides coding for enzymes in a pathway that catalyze a conversion of lactaldehyde to 1,2-propanediol.

In some embodiments of each or any of the above or below mentioned embodiments, the microorganism comprises one or more polynucleotides coding for enzymes in a pathway that catalyzes a conversion of dihydroxyacetone-phosphate to 1-propanol.

In some embodiments of each or any of the above or below mentioned embodiments, the one or more polynucleotides coding for enzymes in a pathway that catalyzes a conversion of dihydroxyacetone-phosphate to 1-propanol include: one or more polynucleotides coding for enzymes in a pathway that catalyze a conversion of dihydroxyacetone-phosphate to methylglyoxal, one or more polynucleotides coding for enzymes in a pathway that catalyze a conversion of methylglyoxal to hydroxyacetone, one or more polynucleotides coding for enzymes in a pathway that catalyze a conversion of hydroxyacetone to 1,2-propanediol, one or more polynucleotides coding for enzymes in a pathway that catalyze a conversion of methylglyoxal to lactaldehyde, one or more polynucleotides coding for enzymes in a pathway that catalyze a conversion of lactaldehyde to 1,2-propanediol, one or more polynucleotides coding for enzymes in a pathway that catalyze a conversion of 1,2-propanediol to propionaldehyde, and/or one or more polynucleotides coding for enzymes in a pathway that catalyze a conversion of propionaldehyde to 1-propanol.

In some embodiments of each or any of the above or below mentioned embodiments, the microorganism comprises one or more polynucleotides coding for enzymes in a pathway that catalyzes a conversion of pyruvate to 1,2-propanediol.

In some embodiments of each or any of the above or below mentioned embodiments, the one or more polynucleotides coding for enzymes in a pathway that catalyzes a conversion of pyruvate to 1,2-propanediol include: one or more polynucleotides coding for enzymes in a pathway that catalyze a conversion of pyruvate to lactate, one or more polynucleotides coding for enzymes in a pathway that catalyze a conversion of lactate to lactoyl-CoA, one or more polynucleotides coding for enzymes in a pathway that catalyze a conversion of lactoyl-CoA to lactaldehyde and/or one or more polynucleotides coding for enzymes in a pathway that catalyze a conversion of lactaldehyde to 1,2-propanediol.

In some embodiments of each or any of the above or below mentioned embodiments, the microorganism comprises one or more polynucleotides coding for enzymes in a pathway that catalyzes a conversion of pyruvate to 1-propanol.

In some embodiments of each or any of the above or below mentioned embodiments, the one or more polynucleotides coding for enzymes in a pathway that catalyzes a conversion of pyruvate to 1-propanol include: one or more polynucleotides coding for enzymes in a pathway that catalyze a conversion of pyruvate to lactate, one or more polynucleotides coding for enzymes in a pathway that catalyze a conversion of lactate to lactoyl-CoA, one or more polynucleotides coding for enzymes in a pathway that catalyze a conversion of lactoyl-CoA to lactaldehyde, one or more polynucleotides coding for enzymes in a pathway that catalyze a conversion of lactaldehyde to 1,2-propanediol, one or more polynucleotides coding for enzymes in a pathway that catalyze a conversion of 1,2-propanediol to propionaldehyde, and/or one or more polynucleotides coding for enzymes in a pathway that catalyze a conversion of propionaldehyde to 1-propanol.

The present disclosure also provides a non-naturally occurring microorganism comprising: a disruption of one or more enzymes that decarboxylate pyruvate and/or a transcription factor of an enzyme that decarboxylates pyruvate; a genetic modification that decreases glucose import into the microorganism; and one or more exogenous polynucleotides encoding one or more enzymes in a pathway that produces cytosolic acetyl-CoA.

In some embodiments of each or any of the above or below mentioned embodiments, the disruption in one or more enzymes that decarboxylate pyruvate and/or a transcription factor of an enzyme that decarboxylates pyruvate results in reduced levels of pyruvate decarboxylase enzymatic activity or no detectable pyruvate decarboxylase enzymatic activity.

In some embodiments of each or any of the above or below mentioned embodiments, the disruption in the one or more enzymes that decarboxylate pyruvate is a deletion or a mutation.

In some embodiments of each or any of the above or below mentioned embodiments, the one or more enzymes that decarboxylate pyruvate include pdc 1, pdc 5, and/or pdc 6. In some embodiments of each or any of the above or below mentioned embodiments, the transcription factor of an enzyme that decarboxylates pyruvate includes pdc2.

In some embodiments of each or any of the above or below mentioned embodiments, the microorganism comprises an exogenous polynucleotide that encodes a transcription factor involved in glucose import.

In some embodiments of each or any of the above or below mentioned embodiments, the microorganism comprises a genetic modification in an endogenous polynucleotide that encodes a transcription factor involved in glucose import.

In some embodiments of each or any of the above or below mentioned embodiments, the genetic modification is a truncation of the MTH1 transcription factor. In an embodiment, the MTH1 transcription factor may have the amino acid sequence as set forth in SEQ ID NO: 1 and the truncated MTH1 transcription factor may have the amino acid sequence set forth in SEQ ID NO: 2.

In some embodiments of each or any of the above or below mentioned embodiments, the truncated MTH1 transcription factor has a longer half-life than an untruncated MTH1 transcription factor.

In some embodiments of each or any of the above or below mentioned embodiments, the one or more exogenous polynucleotides encoding one or more enzymes in a pathway that produces cytosolic acetyl-CoA encode i.) pyruvate formate lyase and pyruvate formate lyase activating enzyme, ii) pyruvate dehydrogenase, dihydrolipoyl transacetylase and dihydrolipoamide dehydrogenase, iii) pyruvate dehydrogenase, dihydrolipoyl transacetylase, dihydrolipoamide dehydrogenase, and pyruvate dehydrogenase complex protein X, or any combination thereof.

In some embodiments of each or any of the above or below mentioned embodiments, the microorganism is a eukaryote.

In some embodiments of each or any of the above or below mentioned embodiments, the eukaryote is a yeast, filamentous fungi, protozoa, or algae.

In some embodiments of each or any of the above or below mentioned embodiments, the microorganism further comprises one or more polynucleotides coding for an acetoacetyl-CoA hydrolase.

In some embodiments of each or any of the above or below mentioned embodiments, the acetoacetyl-CoA hydrolase is produced by introducing a mutation into the polynucleotide that encodes acetoacetyl-CoA:acetate transferase. In some embodiments of each or any of the above or below mentioned embodiments, the mutation is a E51D Glu-Asp mutation corresponding to the numbering of SEQ ID NO: 3.

In some embodiments of each or any of the above or below mentioned embodiments, the microorganism comprises one or more exogenous polynucleotides encoding one or more enzymes in pathways for the co-production of 1,2-propanediol or 1-propanol and 2-propanol from a fermentable carbon source under anaerobic conditions.

In some embodiments of each or any of the above or below mentioned embodiments, the microorganism comprises one or more polynucleotides coding for enzymes in a pathway that catalyzes a conversion of acetyl-CoA to 2-propanol.

In some embodiments of each or any of the above or below mentioned embodiments, the one or more polynucleotides coding for enzymes in a pathway that catalyzes a conversion of acetyl-CoA to 2-propanol include: one or more polynucleotides coding for enzymes in a pathway that catalyze a conversion of acetyl-CoA to acetoacetyl-CoA, one or more polynucleotides coding for enzymes in a pathway that catalyze a conversion of acetoacetyl-CoA to acetoacetate, one or more polynucleotides coding for enzymes in a pathway that catalyze a conversion of acetoacetate to acetone, and/or one or more polynucleotides coding for enzymes in a pathway that catalyze a conversion of acetone to 2-propanol.

In some embodiments of each or any of the above or below mentioned embodiments, the microorganism comprises one or more polynucleotides coding for enzymes in a pathway that catalyzes a conversion of dihydroxyacetone-phosphate to 1,2-propanediol.

In some embodiments of each or any of the above or below mentioned embodiments, the one or more polynucleotides coding for enzymes in a pathway that catalyzes a conversion of dihydroxyacetone-phosphate to 1,2-propanediol include: one or more polynucleotides coding for enzymes in a pathway that catalyze a conversion of dihydroxyacetone-phosphate to methylglyoxal, one or more polynucleotides coding for enzymes in a pathway that catalyze a conversion of methylglyoxal to hydroxyacetone, one or more polynucleotides coding for enzymes in a pathway that catalyze a conversion of hydroxyacetone to 1,2-propanediol, one or more polynucleotides coding for enzymes in a pathway that catalyze a conversion of methylglyoxal to lactaldehyde and/or one or more polynucleotides coding for enzymes in a pathway that catalyze a conversion of lactaldehyde to 1,2-propanediol.

In some embodiments of each or any of the above or below mentioned embodiments, the microorganism comprises one or more polynucleotides coding for enzymes in a pathway that catalyzes a conversion of pyruvate to 1,2-propanediol.

In some embodiments of each or any of the above or below mentioned embodiments, the one or more polynucleotides coding for enzymes in a pathway that catalyzes a conversion of pyruvate to 1,2-propanediol include: one or more polynucleotides coding for enzymes in a pathway that catalyze a conversion of pyruvate to lactate, one or more polynucleotides coding for enzymes in a pathway that catalyze a conversion of lactate to lactoyl-CoA, one or more polynucleotides coding for enzymes in a pathway that catalyze a conversion of lactoyl-CoA to lactaldehyde ando/or one or more polynucleotides coding for enzymes in a pathway that catalyze a conversion of lactaldehyde to 1,2-propanediol.

In some embodiments of each or any of the above or below mentioned embodiments, the microorganism further comprises one or more polynucleotides coding for enzymes in a pathway that catalyzes a conversion of 1,2-propanediol to propanaldehyde.

In some embodiments of each or any of the above or below mentioned embodiments, the enzyme is a B12-independent dehydratase.

In some embodiments of each or any of the above or below mentioned embodiments, the B12-independent dehydratase is from Clostridium butyricum, or Roseburia inulinivorans.

In some embodiments of each or any of the above or below mentioned embodiments, the microorganism comprises one or more polynucleotides coding for enzymes in a pathway that catalyzes a conversion of dihydroxyacetone-phosphate to 1-propanol.

In some embodiments of each or any of the above or below mentioned embodiments, the one or more polynucleotides coding for enzymes in a pathway that catalyzes a conversion of dihydroxyacetone-phosphate to 1-propanol include: one or more polynucleotides coding for enzymes in a pathway that catalyze a conversion of dihydroxyacetone-phosphate to methylglyoxal, one or more polynucleotides coding for enzymes in a pathway that catalyze a conversion of methylglyoxal to hydroxyacetone, one or more polynucleotides coding for enzymes in a pathway that catalyze a conversion of hydroxyacetone to 1,2-propanediol, one or more polynucleotides coding for enzymes in a pathway that catalyze a conversion of methylglyoxal to lactaldehyde, one or more polynucleotides coding for enzymes in a pathway that catalyze a conversion of lactaldehyde to 1,2-propanediol, one or more polynucleotides coding for enzymes in a pathway that catalyze a conversion of 1,2-propanediol to propionaldehyde, and/or one or more polynucleotides coding for enzymes in a pathway that catalyze a conversion of propionaldehyde to 1-propanol.

In some embodiments of each or any of the above or below mentioned embodiments, the microorganism comprises one or more polynucleotides coding for enzymes in a pathway that catalyzes a conversion of pyruvate to 1-propanol.

In some embodiments of each or any of the above or below mentioned embodiments, the one or more polynucleotides coding for enzymes in a pathway that catalyzes a conversion of pyruvate to 1-propanol include: one or more polynucleotides coding for enzymes in a pathway that catalyze a conversion of pyruvate to lactate, one or more polynucleotides coding for enzymes in a pathway that catalyze a conversion of lactate to lactoyl-CoA, one or more polynucleotides coding for enzymes in a pathway that catalyze a conversion of lactoyl-CoA to lactaldehyde, one or more polynucleotides coding for enzymes in a pathway that catalyze a conversion of lactaldehyde to 1,2-propanediol, one or more polynucleotides coding for enzymes in a pathway that catalyze a conversion of 1,2-propanediol to propionaldehyde, and/or one or more polynucleotides coding for enzymes in a pathway that catalyze a conversion of propionaldehyde to 1-propanol.

The present disclosure also provides a non-naturally occurring microorganism comprising: one or more exogenous polynucleotides encoding one or more enzymes in a pathway that produces cytosolic acetyl-CoA; one or more polynucleotides coding for enzymes that produce 1,2-propanediol, and wherein the microorganism has reduced levels of pyruvate decarboxylase enzymatic activity, and wherein the microorganism is capable of growing on a C6 sugar as a sole carbon source and under anaerobic conditions.

In some embodiments of each or any of the above or below mentioned embodiments, the microorganism further comprises one or more polynucleotides encoding one or more enzymes in a pathway that produces acetate.

In some embodiments of each or any of the above or below mentioned embodiments, the microorganism further comprises one or more polynucleotides encoding an acetyl-CoA hydrolase.

In some embodiments of each or any of the above or below mentioned embodiments, the microorganism further comprises one or more polynucleotides encoding a phosphate acetyltransferase and acetyl-phosphate kinase.

In some embodiments of each or any of the above or below mentioned embodiments, the microorganism further comprises one or more polynucleotides encoding a lactate CoA-transferase.

In some embodiments of each or any of the above or below mentioned embodiments, the microorganism comprises one or more polynucleotides coding for enzymes in a pathway that catalyzes a conversion of 1,2-propanediol to 1-propanol.

In some embodiments of each or any of the above or below mentioned embodiments, the one or more polynucleotides coding for enzymes in a pathway that catalyzes a conversion of 1,2-propanediol to 1-propanol include: one or more polynucleotides coding for enzymes in a pathway that catalyze a conversion of 1,2-propanediol to propionaldehyde, and/or one or more polynucleotides coding for enzymes in a pathway that catalyze a conversion of propionaldehyde to 1-propanol.

In some embodiments of each or any of the above or below mentioned embodiments, the microorganism has no detectable pyruvate decarboxylase enzymatic activity.

In some embodiments of each or any of the above or below mentioned embodiments, the microorganism has a disruption in one or more polynucleotides that code for one or more enzymes that decarboxylate pyruvate.

In some embodiments of each or any of the above or below mentioned embodiments, the microorganism has a disruption in one or more polynucleotides that code for one or more enzymes that decarboxylate pyruvate (e.g., a pyruvate decarboxylase) or a disruption in one or more polynucleotides that code for a transcription factor of an enzyme that decarboxylates pyruvate.

In some embodiments of each or any of the above or below mentioned embodiments, the disruption in the one or more polynucleotides is a deletion or a mutation.

In some embodiments of each or any of the above or below mentioned embodiments, the one or more polynucleotides that code for enzymes that decarboxylate pyruvate code for pdc1, pdc5, and/or pdc6. In some embodiments of each or any of the above or below mentioned embodiments, the one or more polynucleotides that code for a transcription factor of one or more enzymes that decarboxylates pyruvate code for pdc2.

In some embodiments of each or any of the above or below mentioned embodiments, the microorganism comprises an exogenous polynucleotide that encodes a transcription factor involved in glucose import.

In some embodiments of each or any of the above or below mentioned embodiments, the microorganism comprises a genetic modification in an endogenous polynucleotide that encodes a transcription factor involved in glucose import.

In some embodiments of each or any of the above or below mentioned embodiments, the genetic modification is a truncation of the MTH1 transcription factor. In an embodiment, the MTH1 transcription factor may have the amino acid sequence as set forth in SEQ ID NO: 1 and the truncated MTH1 transcription factor may have the amino acid sequence set forth in SEQ ID NO: 2.

In some embodiments of each or any of the above or below mentioned embodiments, the truncated MTH1 transcription factor has a longer half-life than an untruncated MTH1 transcription factor.

The present disclosure also provides a non-naturally occurring microorganism comprising: one or more exogenous polynucleotides encoding one or more enzymes in a pathway that produces cytosolic acetyl-CoA; one or more polynucleotides coding for an acetyl-CoA acetyltransferase; one or more polynucleotides coding for enzymes that produce 1,2-propanediol, wherein the microorganism has reduced levels of pyruvate decarboxylase enzymatic activity, and wherein the microorganism is capable of growing on a C6 sugar as a sole carbon source and under anaerobic conditions.

In some embodiments of each or any of the above or below mentioned embodiments, the microorganism further comprises one or more polynucleotides encoding one or more enzymes in a pathway that produces 1-propanol.

In some embodiments of each or any of the above or below mentioned embodiments, the microorganism further comprises one or more polynucleotides coding for an acetoacetyl-CoA hydrolase.

In some embodiments of each or any of the above or below mentioned embodiments, the microorganism further comprises one or more polynucleotides encoding one or more enzymes in a pathway that produces 2-propanol.

In some embodiments of each or any of the above or below mentioned embodiments, the microorganism further comprises one or more polynucleotides coding for a HMG-CoA synthase and HMG-CoA lyase (see, e.g., WO2014076232).

In some embodiments of each or any of the above or below mentioned embodiments, the microorganism has no detectable pyruvate decarboxylase enzymatic activity.

In some embodiments of each or any of the above or below mentioned embodiments, the microorganism has a disruption in one or more polynucleotides that code for one or more enzymes that decarboxylate pyruvate.

In some embodiments of each or any of the above or below mentioned embodiments, the microorganism has a disruption in each of the one or more polynucleotides that code for enzymes that decarboxylate pyruvate.

In some embodiments of each or any of the above or below mentioned embodiments, the disruption in the one or more polynucleotides is a deletion or a mutation.

In some embodiments of each or any of the above or below mentioned embodiments, the one or more polynucleotides code for pyruvate decarboxylase 1, 5, and/or 6.

In some embodiments of each or any of the above or below mentioned embodiments, the microorganism comprises an exogenous polynucleotide that encodes a transcription factor involved in glucose import.

In some embodiments of each or any of the above or below mentioned embodiments, the microorganism comprises a genetic modification in an endogenous polynucleotide that encodes a transcription factor involved in glucose import.

In some embodiments of each or any of the above or below mentioned embodiments, the genetic modification is a truncation of the MTH1 transcription factor. In an embodiment, the MTH1 transcription factor may have the amino acid sequence as set forth in SEQ ID NO: 1 and the truncated MTH1 transcription factor may have the amino acid sequence set forth in SEQ ID NO: 2.

In some embodiments of each or any of the above or below mentioned embodiments, the truncated MTH1 transcription factor has a longer half-life than an untruncated MTH1 transcription factor.

In some embodiments of each or any of the above or below mentioned embodiments, the microorganism comprises one or more polynucleotides coding for enzymes in a pathway that catalyzes a conversion of acetyl-CoA to 2-propanol.

In some embodiments of each or any of the above or below mentioned embodiments, the one or more polynucleotides coding for enzymes in a pathway that catalyzes a conversion of acetyl-CoA to 2-propanol include: one or more polynucleotides coding for enzymes in a pathway that catalyze a conversion of acetyl-CoA to acetoacetyl-CoA, one or more polynucleotides coding for enzymes in a pathway that catalyze a conversion of acetoacetyl-CoA to acetoacetate, one or more polynucleotides coding for enzymes in a pathway that catalyze a conversion of acetoacetate to acetone, and/or one or more polynucleotides coding for enzymes in a pathway that catalyze a conversion of acetone to 2-propanol.

In some embodiments of each or any of the above or below mentioned embodiments, the microorganism comprises one or more polynucleotides coding for enzymes in a pathway that catalyzes a conversion of 1,2-propanediol to 1-propanol.

In some embodiments of each or any of the above or below mentioned embodiments, the one or more polynucleotides coding for enzymes in a pathway that catalyzes a conversion of 1,2-propanediol to 1-propanol include: one or more polynucleotides coding for enzymes in a pathway that catalyze a conversion of 1,2-propanediol to propionaldehyde, and/or one or more polynucleotides coding for enzymes in a pathway that catalyze a conversion of propionaldehyde to 1-propanol.

The present disclosure also provides a non-naturally occurring microorganism comprising: one or more exogenous polynucleotides encoding one or more enzymes in a pathway that produces cytosolic acetyl-CoA; one or more polynucleotides coding for an acetoacetyl-CoA hydrolase; one or more polynucleotides coding for enzymes in a pathway that catalyzes a conversion of dihydroxyacetone phosphate to 1,2-propanediol or 1-propanol or one or more polynucleotides coding for enzymes in a pathway that catalyzes a conversion of lactate to 1,2-propanediol or 1-propanol, and one or more polynucleotides coding for enzymes in a pathway that catalyzes a conversion of acetyl-CoA to 2-propanol, wherein the microorganism has reduced levels of pyruvate decarboxylase enzymatic activity, and wherein the microorganism is capable of growing on a C6 sugar as a sole carbon source and under anaerobic conditions.

In some embodiments of each or any of the above or below mentioned embodiments, the microorganism has no detectable pyruvate decarboxylase enzymatic activity.

In some embodiments of each or any of the above or below mentioned embodiments, the microorganism has a disruption in one or more polynucleotides that code for one or more enzymes that decarboxylate pyruvate (e.g., a pyruvate decarboxylase) or a disruption in one or more polynucleotides that code for a transcription factor of an enzyme that decarboxylates pyruvate.

In some embodiments of each or any of the above or below mentioned embodiments, the microorganism has a disruption in each of the one or more polynucleotides that code for enzymes that decarboxylate pyruvate or a disruption in each of the polynucleotides that code for a transcription factor of an enzyme that decarboxylates pyruvate.

In some embodiments of each or any of the above or below mentioned embodiments, the disruption in the one or more polynucleotides is a deletion or a mutation.

In some embodiments of each or any of the above or below mentioned embodiments, the one or more polynucleotides that code for enzymes that decarboxylate pyruvate code for pdc1, pdc5, and/or pdc6. In some embodiments of each or any of the above or below mentioned embodiments, the one or more polynucleotides that code for a transcription factor of one or more enzymes that decarboxylates pyruvate code for pdc2.

In some embodiments of each or any of the above or below mentioned embodiments, the microorganism comprises an exogenous polynucleotide that encodes a transcription factor involved in glucose import.

In some embodiments of each or any of the above or below mentioned embodiments, the microorganism comprises a genetic modification in an endogenous polynucleotide that encodes a transcription factor involved in glucose import.

In some embodiments of each or any of the above or below mentioned embodiments, the genetic modification is a truncation of the MTH1 transcription factor. In an embodiment, the MTH1 transcription factor may have the amino acid sequence as set forth in SEQ ID NO: 1 and the truncated MTH1 transcription factor may have the amino acid sequence set forth in SEQ ID NO: 2.

In some embodiments of each or any of the above or below mentioned embodiments, the truncated MTH1 transcription factor has a longer half-life than an untruncated MTH1 transcription factor.

In some embodiments of each or any of the above or below mentioned embodiments, the one or more polynucleotides coding for enzymes in a pathway that catalyzes a conversion of lactate to 1,2-propanediol include: one or more polynucleotides coding for enzymes in a pathway that catalyze a conversion of lactate to lactoyl-CoA, one or more polynucleotides coding for enzymes in a pathway that catalyze a conversion of lactoyl-CoA to lactaldehyde and/or one or more polynucleotides coding for enzymes in a pathway that catalyze a conversion of lactaldehyde to 1,2-propanediol.

In some embodiments of each or any of the above or below mentioned embodiments, the one or more polynucleotides coding for enzymes in a pathway that catalyzes a conversion of dihydroxyacetone-phosphate to 1,2-propanodiol include: one or more polynucleotides coding for enzymes in a pathway that catalyze a conversion of dihydroxyacetone-phosphate to methylglyoxal, one or more polynucleotides coding for enzymes in a pathway that catalyze a conversion of methylglyoxal to hydroxyacetone, one or more polynucleotides coding for enzymes in a pathway that catalyze a conversion of hydroxyacetone to 1,2-propanediol and/or one or more polynucleotides coding for enzymes in a pathway that catalyze a conversion of methylglyoxal to lactaldehyde, one or more polynucleotides coding for enzymes in a pathway that catalyze a conversion of lactaldehyde to 1,2-propanediol.

In some embodiments of each or any of the above or below mentioned embodiments, the one or more polynucleotides coding for enzymes in a pathway that catalyzes a conversion of lactate to 1-propanol include: one or more polynucleotides coding for enzymes in a pathway that catalyze a conversion of lactate to lactoyl-CoA, one or more polynucleotides coding for enzymes in a pathway that catalyze a conversion of lactoyl-CoA to lactaldehyde, one or more polynucleotides coding for enzymes in a pathway that catalyze a conversion of lactaldehyde to 1,2-propanediol, one or more polynucleotides coding for enzymes in a pathway that catalyze a conversion of 1,2-propanediol to propionaldehyde, and/or one or more polynucleotides coding for enzymes in a pathway that catalyze a conversion of propionaldehyde to 1-propanol.

In some embodiments of each or any of the above or below mentioned embodiments, the one or more polynucleotides coding for enzymes in a pathway that catalyzes a conversion of dihydroxyacetone-phosphate to 1-propanol include: one or more polynucleotides coding for enzymes in a pathway that catalyze a conversion of dihydroxyacetone-phosphate to methylglyoxal, one or more polynucleotides coding for enzymes in a pathway that catalyze a conversion of methylglyoxal to hydroxyacetone, one or more polynucleotides coding for enzymes in a pathway that catalyze a conversion of hydroxyacetone to 1,2-propanediol, one or more polynucleotides coding for enzymes in a pathway that catalyze a conversion of methylglyoxal to lactaldehyde, one or more polynucleotides coding for enzymes in a pathway that catalyze a conversion of lactaldehyde to 1,2-propanediol, one or more polynucleotides coding for enzymes in a pathway that catalyze a conversion of 1,2-propanediol to propionaldehyde, and/or one or more polynucleotides coding for enzymes in a pathway that catalyze a conversion of propionaldehyde to 1-propanol.

In some embodiments of each or any of the above or below mentioned embodiments, the one or more polynucleotides coding for enzymes in a pathway that catalyzes a conversion of acetyl-CoA to 2-propanol include: one or more polynucleotides coding for enzymes in a pathway that catalyze a conversion of acetyl-CoA to acetoacetyl-CoA, one or more polynucleotides coding for enzymes in a pathway that catalyze a conversion of acetoacetyl-CoA to acetoacetate, one or more polynucleotides coding for enzymes in a pathway that catalyze a conversion of acetoacetate to acetone, and/or one or more polynucleotides coding for enzymes in a pathway that catalyze a conversion of acetone to 2-propanol.

C6 sugar as a sole carbon source

The present disclosure also provides a non-naturally occurring microorganism comprising: a disruption of one or more enzymes that decarboxylate pyruvate; a genetic modification that permits growth of the microorganism on a C6 molecule as a sole carbon source; one or more exogenous polynucleotides encoding one or more enzymes in a pathway that produces cytosolic acetyl-CoA, one or more polynucleotides coding for an acetoacetyl-CoA hydrolase, one or more polynucleotides coding for enzymes in a pathway that catalyzes a conversion of dihydroxyacetone phosphate to 1,2-propanediol or 1-propanol, and one or more polynucleotides coding for enzymes in a pathway that catalyzes a conversion of acetyl-CoA to 2-propanol, and optionally one or more polynucleotides coding for enzymes in a pathway that catalyzes a conversion of lactate to 1,2-propanediol or 1-propanol.

In some embodiments of each or any of the above or below mentioned embodiments, the microorganism has no detectable pyruvate decarboxylase enzymatic activity.

In some embodiments of each or any of the above or below mentioned embodiments, the microorganism has a disruption in one or more polynucleotides that code for one or more enzymes that decarboxylate pyruvate (e.g., a pyruvate decarboxylase) or a disruption in one or more polynucleotides that code for a transcription factor of an enzyme that decarboxylates pyruvate.

In some embodiments of each or any of the above or below mentioned embodiments, the microorganism has a disruption in each of the one or more polynucleotides that code for enzymes that decarboxylate pyruvate or a disruption in each of the polynucleotides that code for a transcription factor of an enzyme that decarboxylates pyruvate.

In some embodiments of each or any of the above or below mentioned embodiments, the disruption in the one or more polynucleotides is a deletion or a mutation.

In some embodiments of each or any of the above or below mentioned embodiments, the one or more polynucleotides that code for enzymes that decarboxylate pyruvate code for pdc1, pdc5, and/or pdc6. In some embodiments of each or any of the above or below mentioned embodiments, the one or more polynucleotides that code for a transcription factor of one or more enzymes that decarboxylates pyruvate code for pdc2.

In some embodiments of each or any of the above or below mentioned embodiments, the microorganism comprises an exogenous polynucleotide that encodes a transcription factor involved in glucose import.

In some embodiments of each or any of the above or below mentioned embodiments, the microorganism comprises a genetic modification in an endogenous polynucleotide that encodes a transcription factor involved in glucose import.

In some embodiments of each or any of the above or below mentioned embodiments, the genetic modification is a truncation of the MTH1 transcription factor. In an embodiment, the MTH1 transcription factor may have the amino acid sequence as set forth in SEQ ID NO: 1 and the truncated MTH1 transcription factor may have the amino acid sequence set forth in SEQ ID NO: 2.

In some embodiments of each or any of the above or below mentioned embodiments, the truncated MTH1 transcription factor has a longer half-life than an untruncated MTH1 transcription factor.

In some embodiments of each or any of the above or below mentioned embodiments, the one or more polynucleotides coding for enzymes in a pathway that catalyzes a conversion of dihydroxyacetone-phosphate to 1,2-propanediol include: one or more polynucleotides coding for enzymes in a pathway that catalyze a conversion of dihydroxyacetone-phosphate to methylglyoxal, one or more polynucleotides coding for enzymes in a pathway that catalyze a conversion of methylglyoxal to hydroxyacetone, one or more polynucleotides coding for enzymes in a pathway that catalyze a conversion of hydroxyacetone to 1,2-propanediol, one or more polynucleotides coding for enzymes in a pathway that catalyze a conversion of methylglyoxal to lactaldehyde and/or one or more polynucleotides coding for enzymes in a pathway that catalyze a conversion of lactaldehyde to 1,2-propanediol.

In some embodiments of each or any of the above or below mentioned embodiments, the one or more polynucleotides coding for enzymes in a pathway that catalyzes a conversion of dihydroxyacetone-phosphate to 1-propanol include: one or more polynucleotides coding for enzymes in a pathway that catalyze a conversion of dihydroxyacetone-phosphate to methylglyoxal, one or more polynucleotides coding for enzymes in a pathway that catalyze a conversion of methylglyoxal to hydroxyacetone, one or more polynucleotides coding for enzymes in a pathway that catalyze a conversion of hydroxyacetone to 1,2-propanediol, one or more polynucleotides coding for enzymes in a pathway that catalyze a conversion of methylglyoxal to lactaldehyde, one or more polynucleotides coding for enzymes in a pathway that catalyze a conversion of lactaldehyde to 1,2-propanediol, one or more polynucleotides coding for enzymes in a pathway that catalyze a conversion of 1,2-propanediol to propionaldehyde, and/or one or more polynucleotides coding for enzymes in a pathway that catalyze a conversion of propionaldehyde to 1-propanol.

In some embodiments of each or any of the above or below mentioned embodiments, the one or more polynucleotides coding for enzymes in a pathway that catalyzes a conversion of acetyl-CoA to 2-propanol include: one or more polynucleotides coding for enzymes in a pathway that catalyze a conversion of acetyl-CoA to acetoacetyl-CoA, one or more polynucleotides coding for enzymes in a pathway that catalyze a conversion of acetoacetyl-CoA to acetoacetate, one or more polynucleotides coding for enzymes in a pathway that catalyze a conversion of acetoacetate to acetone, and/or one or more polynucleotides coding for enzymes in a pathway that catalyze a conversion of acetone to 2-propanol.

The present disclosure also provides methods for co-producing 1,2-propanediol or 1-propanol and 2-propanol from a fermentable carbon source under anaerobic conditions, the method comprising: a.) providing a fermentable carbon source in substantially anaerobic culture media; and b.) contacting the fermentable carbon source with any of the non-naturally occurring microorganisms disclosed herein in a fermentation media, wherein the microorganism co-produces 1,2-propanediol or 1-propanol and 2-propanol from the fermentable carbon source.

In some embodiments of each or any of the above or below mentioned embodiments, the fermentable carbon source is sugarcane juice, sugarcane molasses, hydrolyzed starch, hydrolyzed lignocellulosic materials, glucose, sucrose, fructose, lactate, lactose, xylose, pyruvate, or glycerol in any form or mixture thereof.

In some embodiments of each or any of the above or below mentioned embodiments, the fermentable carbon source is a monosaccharide, oligosaccharide, or polysaccharide.

The present disclosure also provides methods of making a non-naturally occurring microorganism that lacks pyruvate decarboxylase enzymatic activity, that is capable of growth on a C6 molecule as a sole carbon source, and that is capable of producing 1,2-propanediol or 1-propanol and 2-propanol from a fermentable carbon source under anaerobic conditions, the method comprising: introducing a disruption in one or more polynucleotides in the microorganism that encode enzymes that decarboxylate pyruvate; introducing a genetic modification in the microorganism that decreases import of glucose into the microorganism; introducing into the microorganism one or more exogenous polynucleotides encoding one or more enzymes in a pathway that produces cytosolic acetyl-CoA; introducing into the microorganism one or more polynucleotides coding for an acetoacetyl-CoA hydrolase or acetoacetyl-Coa transferase; introducing into the microorganism one or more polynucleotides coding for enzymes in a pathway that catalyzes a conversion of dihydroxyacetone phosphate or pyruvate to 1,2-propanediol or 1-propanol, and introducing into the microorganism one or more polynucleotides coding for enzymes in a pathway that catalyzes a conversion of acetyl-CoA to 2-propanol, and optionally introducing into the microorganism one or more polynucleotides coding for enzymes in a pathway that catalyzes a conversion of lactate to 1,2-propanediol or 1-propanol.

### BRIEF DESCRIPTION OF THE DRAWINGS

The foregoing summary, as well as the following detailed description of the disclosure, will be better understood when read in conjunction with the appended figures. For the purpose of illustrating the disclosure, one preferred embodiment is shown in the following figure. It should be understood, however, that the disclosure is not limited to the precise arrangements, examples and instrumentalities shown.

FIG. 1 depicts an exemplary pathway for the co-production of 2-propanol and 1,2-propanediol, where 1,2-propanediol is produced via a dihydroxyacetone-phosphate intermediate.

FIG. 2 depicts an exemplary pathway for the co-production of 1,2-propanediol and 2-propanol, where 1,2-propanediol is produced via a glyceraldehyde 3-phosphate intermediate.

FIG. 3 depicts an exemplary pathway for the co-production of 2-propanol and 1-propanol, where 1-propanol is produced via a dihydroxyacetone-phosphate intermediate.

FIG. 4 depicts an exemplary pathway for the co-production of 2-propanol and 1-propanol, where 1-propanol is produced via a glyceraldehyde-3-phosphate intermediate.

FIG. 5 depicts a schematic representation of the anaerobic flask system used in Example 1.

FIG. 6A-C summarizes the results of Example 1 showing restored anaerobic growth of the BRKY-272 strain compared to the control (BRKY-397), carbon source consumption and metabolite profile. FIG. 6A shows increased growth of the BRKY-272 strain compared to the control (BRKY-397) in the anaerobic flask system shown in FIG. 5. FIGS. 6B and FIG. 6C show the kynetics of glucose consumption and metabolite production in strains BRKY-397 (control) and BRKY-272, respectively.

FIG. 7 summarizes the results of Example 3 showing co-production of 2-propanol and 1,2-propanediol in an ethanol-null strain BRKY-399.

### DETAILED DESCRIPTION

The present disclosure generally relates to microorganisms (e.g., non-naturally occurring microorganisms) that comprise a genetically modified pathway and uses of the microorganisms for the conversion of a fermentable carbon source to 2-propanol and 1-propanol and/or 1,2-propanediol. Such microorganisms may comprise one or more polynucleotides coding for enzymes that catalyze a conversion of a fermentable carbon source to 2-propanol, one or more polynucleotides coding for enzymes that catalyze a conversion of a fermentable carbon source to 1,2-propanediol, one or more polynucleotides coding for enzymes that catalyse a conversion of 1,2-propanediol to 1-propanol.

This disclosure provides, in part, the discovery of novel anaerobic enzymatic pathways including, for example, novel combinations of enzymatic pathways, for the production of 2-propanol and 1-propanol and/or 1,2-propanediol from a carbon source (e.g., a fermentable carbon source).

The present disclosure provides microorganisms (e.g., S. cerevisiae) for the co-production of 2-propanol and 1-propanol and/or 1,2-propanediol. Microorganisms may be modified so that they may co-produce 2-propanol and 1-propanol and/or 1,2-propanediol. In an embodiment, a microorganism may have its native ethanol production reduced or elimiated (i.e., shut off). In an embodiment, to eliminate ethanol production in the microorganism the activity of pyruvate decarboxylase (i.e., the enzyme which decarboxylates pyruvate and in the process makes acetaldehyde and CO₂) may be disrupted including, for example, knocked-out. Pyruvate decarboxylase comes in three isoforms in yeast and its activity can be mostly knocked out by deleting the genes PDC1, PDC5, and PDC6. Without wishing to be bound by a theory of the invention, the elimination of the pyruvate decarboxylase activity in the cell's cytoplasm renders the yeast cell unable to grow under anaerobic conditions due to two factors: (1) the lack of an alternative route for cytoplasmic acetyl-CoA production, due to the lack of acetaldehyde that would be converted to acetate and acetyl-coA; and (2) a redox imbalance due to excess NADH because the NADH is no longer oxidized in the conversion of acetaldehyde to ethanol. Thus, it is necessary to also alter the ability of the microorgansim to import glucose by truncating a transcription factor of the glucose importer called MTH1. This truncation then restores the ability of the ΔPDC1,5,6 mutant microorganism to survive on C6 sugars. In an embodiment, one or more polynucleotides coding for a bacterial pyruvate formate lyase or cytosolic pyruvate dehydrogenase complex may be inserted into the microorganism to convert pyruvate into Acetyl CoA in the cytosol. In an embodiment, the microorganism may be modified to comprise one or more polynucleotides that code for enzymes in a pathway for the coproduction of 2-propanol and 1-propanol and/or 1,2-propanediol. In a further embodiment, the microorganism may be modified to comprise an acetoacetylCoA hydrolase. Such an acetoacetylCoA hydrolase may be engineered from an acetoacetylCoA:acetate transferase by making a single Glu-Asp mutation in the acetoacetylCoA:acetate transferase (e.g., a E51 D Glu-Asp mutation corresponding to the numbering of SEQ ID NO: 3). In an additional embodiment, a microorganism may be modified to comprise one or more polynucleotides coding for a B12-independent dehydratase from the organism Roseburia inuvolurans to convert 1,2-propanediol to propanaldehyde. Microorganims that comprise one or more of the modifications set forth above are termed a non-naturally occuring microroganism or a modified microorganism.

WO2004099425 discloses the overproduction of pyruvate in S. cerevisiae by knocking out pyruvate decarboxylase activity and a directed evolution process that allowed this triple mutant to grow on glucose due to a truncation of the MTH1 transcription factor. However, the scope stopped at the overproduction of pyruvate in aerobic fermentation systems. The use of oxygen, in this context, was essential as there is a huge buildup of NADH in the cell due to the fact that NADH is no longer oxidized in the conversion of acetaldehyde to ethanol.

The present disclosure further comprises a pyruvate overproducing cell able to produce cytosolic Acetyl-CoA inserting for example, bacterial pyruvate formate lyase or cytosolic pyruvate dehydrogenase complex to convert pyruvate into Acetyl-CoA in the cytosol of the eukaryote cell. The insertion of pyruvate formate lyase in to a PDC-negative yeast strain was disclosed by Waks and Silver in Engineering a Synthetic Dual-Organism System for Hydrogen Production (Applied and Environmental Microbiology, vol. 75, n. 7, 2009, p. 1867-1875) without success in anaerobic growth or metabolism. Furthermore, the present disclosure further comprises a pyruvate overproducing cell able to produce cytosolic Acetyl-CoA and to grow under anaerobic conditions by providing a temporary redox sink that allows reoxidation of NADH by introducing a gene coding for a bacterial soluble NAD(P)+ transhydrogenase (Si-specific) (udhA gene from E. coli, E.C. number 1.6.1.1.) that catalyzes the interconversion of NADP++NADH=NADPH+NAD+. The concomitant expression of the PFL and udhA enzymes to restore anaerobic growth to the PDC-null yeast strain expressing the truncated MTH1 constitutes the first report of anaerobic growth of a PDC-null yeast strain and serves as a new eukaryotic chassis for the production of commodity chemicals.

Moreover, the present disclosure teaches how to make the 1,2-propanol or 1-propanol and 2-propanol pathways work in the new eukaryote chassis. Since the cell had the production of acetaldehyde knocked out, acetate is no longer formed and a new CoA receptor is necessary for the 2-propanol metabolic pathway to work. To solve this matter, the present disclosure proposes, for example, to engineer an acetoacetyl-CoA hydrolase from an acetoacetyl-CoA:acetate transferase (EC number 2.8.3.8.) by applying a mutation to it that was reported by Mack and Buckel in Conversion of glutaconate CoA-transferase from Acidaminococcus fermentans into an acyl-CoA hydrolase by site-directed mutagenesis (FEBS Letters, v. 405, n. 2, 1997, p. 209-212) but applied to another transferase. In that case, the “glucatonate CoA transferase” was transformed into a hydrolase by a single Glu-Asp mutation. The main advantage of this strategy is that the specificity of the enzyme for acetoacetyl-CoA is maintained since the transferase activity of a protein that already has high specificity for acetoacetyl-CoA is knocked out. The methods provided herein may also provide end-results similar to those of sterilization without the high capital expenditure and continuing higher management costs required to establish and maintain sterility throughout a production process. In this regard, most industrial-scale isoprene production processes are operated in the presence of measurable numbers of bacterial contaminants. Such drawbacks of prior methods are avoided by the presently disclosed methods as the toxic nature of the produced 2-propanol and/or 1-propanol reduce contaminants in the production process.

Additionally, the non-naturally occurring eukaryotic microorganism disclosed herein is capable of anaerobic growth and concomitant production of 2-propanol and 1-propanol and/or 1,2-propanediol. The supplementation of oxygen and nitrogen in a fermenter requires an additional investment for aerobic process. Additionally, aerobic fermentation processes for the production of 2-propanol and 1-propanol and/or 1,2-propanediol present several drawbacks at industrial scale (where it is technically challenging to maintain aseptic conditions) such as the fact that: (i) greater biomass is obtained reducing overall yields on carbon; (ii) the presence of oxygen favors the growth of contaminants (Weusthuis et al., 2011, Trends in Biotechnology, 2011, Vol. 29, No. 4, 153-158) and (iii) the mixture of oxygen and gaseous compounds poses serious risks of explosion, (iv) the oxygen can catalyze the unwanted reaction of polymerization of the olefinic compounds and, finally, (v) higher costs of fermentation and purification in aerobic conditions. Each of the drawbacks associated with aerobic fermentation including, for example, the risk of an explosion during the manufacture of 2-propanol and 1-propanol and/or 1,2-propanediol including dilution by oxygen and nitrogen are overcome by the anaerobic fermentation methods provided herein.

The present disclosure provides microorganisms comprising one or more polynucleotides coding for enzymes in a pathway that catalyzes a conversion of the fermentable carbon source to one or more intermediates in a pathway for the co-production of 2-propanol and 1-propanol and/or 1,2-propanediol, and one or more polynucleotides coding for enzymes in a pathway that catalyze a conversion of the one or more intermediates to 2-propanol and 1-propanol and/or 1,2-propanediol in a fermentation media, wherein 1,2-propanediol and 1-propanol are produced via a dihydroxyacetone phosphate intermediate or a pyruvate intermediate. In some embodiments, 2-propanol is produced via an acetyl-CoA intermediate.

The present disclosure also provides methods of co-producing 2-propanol and 1-propanol and/or 1,2-propanediol from a fermentable carbon source by providing a fermentable carbon source; contacting the fermentable carbon source with a microorganism comprising one or more polynucleotides coding for enzymes in a pathway that catalyzes a conversion of the fermentable carbon source to one or more intermediates in a pathway for the co-production of 2-propanol and 1-propanol and/or 1,2-propanediol, and one or more polynucleotides coding for enzymes in a pathway that catalyze a conversion of the one or more intermediates to 2-propanol and 1-propanol and/or 1,2-propanediol in a fermentation media; and expressing the one or more polynucleotides coding for the enzymes in the pathway that catalyzes a conversion of the fermentable carbon source to one or more intermediates in a pathway for the co-production of 2-propanol and 1-propanol and/or 1,2-propanediol and one or more polynucleotides coding for enzymes in a pathway that catalyze a conversion of the one or more intermediates to 2-propanol and 1-propanol and/or 1,2-propanediol in the microorganism to produce 12-propanol and 1-propanol and/or 1,2-propanediol, wherein 2-propanol and 1-propanol and/or 1,2-propanediol are produced via a dihydroxyacetone phosphate intermediate and/or a pyruvate intermediate, and wherein the co-production method is anaerobic.

It will be understood that the steps involved in any and all of the methods described herein may be performed in any order and are not to be limited or restricted to the order in which they are particularly recited. For example, the present disclosure provides methods of co-producing 2-propanol and 1-propanol and/or 1,2-propanediol from a fermentable carbon source, comprising: providing a fermentable carbon source; contacting the fermentable carbon source with a microorganism comprising one or more polynucleotides coding for enzymes in a pathway that catalyzes a conversion of the fermentable carbon source to one or more intermediates in a pathway for the co-production of 2-propanol and 1-propanol and/or 1,2-propanediol, and one or more polynucleotides coding for enzymes in a pathway that catalyze a conversion of the one or more intermediates to 2-propanol and 1-propanol and/or 1,2-propanediol in a fermentation media; and expressing the one or more polynucleotides coding for the enzymes in the pathway that catalyzes a conversion of the fermentable carbon source to one or more intermediates in a pathway for the co-production of 2-propanol and 1-propanol and/or 1,2-propanediol and one or more polynucleotides coding for enzymes in a pathway that catalyze a conversion of the one or more intermediates to 2-propanol and 1-propanol and/or 1,2-propanediol in the microorganism to produce 2-propanol and 1-propanol and/or 1,2-propanediol. As such, expression of the one or more polynucleotides coding for the enzymes in the pathway that catalyzes a conversion of the fermentable carbon source to one or more intermediates in a pathway for the co-production of 2-propanol and 1-propanol and/or 1,2-propanediol and one or more polynucleotides coding for enzymes in a pathway that catalyze a conversion of the one or more intermediates to 2-propanol and 1-propanol and/or 1,2-propanediol in the microorganism to produce 2-propanol and 1-propanol and/or 1,2-propanediol may be preformed prior to or after contacting the fermentable carbon source with a microorganism comprising one or more polynucleotides coding for enzymes in a pathway that catalyzes a conversion of the fermentable carbon source to one or more intermediates in a pathway for the co-production of 2-propanol and 1-propanol and/or 1,2-propanediol, and one or more polynucleotides coding for enzymes in a pathway that catalyze a conversion of the one or more intermediates to 2-propanol and 1-propanol and/or 1,2-propanediol in a fermentation media.

Any of the intermediates produced in any of the enzymatic pathways disclosed herein may be an intermediate in the classical sense of the word in that they may be enzymatically converted to another intermediate or an end product. Alternatively, the intermediates themselves may be considered an end product.

As used herein, the term “biological activity” or “functional activity,” when referring to a protein, polypeptide or peptide, may mean that the protein, polypeptide or peptide exhibits a functionality or property that is useful as relating to some biological process, pathway or reaction. Biological or functional activity can refer to, for example, an ability to interact or associate with (e.g., bind to) another polypeptide or molecule, or it can refer to an ability to catalyze or regulate the interaction of other proteins or molecules (e.g., enzymatic reactions).

As used herein, the term “culturing” may refer to growing a population of cells, e.g., microbial cells, under suitable conditions for growth, in a liquid or on solid medium.

As used herein, the term “derived from” may encompass the terms originated from, obtained from, obtainable from, isolated from, and created from, and generally indicates that one specified material finds its origin in another specified material or has features that can be described with reference to the another specified material.

As used herein, “exogenous polynucleotide” refers to any deoxyribonucleic acid that originates outside of the microorganism.

As used herein, the term “an expression vector” may refer to a DNA construct containing a polynucleotide or nucleic acid sequence encoding a polypeptide or protein, such as a DNA coding sequence (e.g. gene sequence) that is operably linked to one or more suitable control sequence(s) capable of affecting expression of the coding sequence in a host. Such control sequences include a promoter to affect transcription, an optional operator sequence to control such transcription, a sequence encoding suitable mRNA ribosome binding sites, and sequences which control termination of transcription and translation. The vector may be a plasmid, cosmid, phage particle, bacterial artificial chromosome, or simply a potential genomic insert. Once transformed into a suitable host, the vector may replicate and function independently of the host genome (e.g., independent vector or plasmid), or may, in some instances, integrate into the genome itself (e.g., integrated vector). The plasmid is the most commonly used form of expression vector. However, the disclosure is intended to include such other forms of expression vectors that serve equivalent functions and which are, or become, known in the art.

As used herein, the term “expression” may refer to the process by which a polypeptide is produced based on a nucleic acid sequence encoding the polypeptides (e.g., a gene). The process includes both transcription and translation.

As used herein, the term “gene” may refer to a DNA segment that is involved in producing a polypeptide or protein (e.g., fusion protein) and includes regions preceding and following the coding regions as well as intervening sequences (introns) between individual coding segments (exons).

As used herein, the term “heterologous,” with reference to a nucleic acid, polynucleotide, protein or peptide, may refer to a nucleic acid, polynucleotide, protein or peptide that does not naturally occur in a specified cell, e.g., a host cell. It is intended that the term encompass proteins that are encoded by naturally occurring genes, mutated genes, and/or synthetic genes. In contrast, the term homologous, with reference to a nucleic acid, polynucleotide, protein or peptide, refers to a nucleic acid, polynucleotide, protein or peptide that occurs naturally in the cell.

As used herein, the term a “host cell” may refer to a cell or cell line, including a cell such as a microorganism which a recombinant expression vector may be transfected for expression of a polypeptide or protein (e.g., fusion protein). Host cells include progeny of a single host cell, and the progeny may not necessarily be completely identical (in morphology or in total genomic DNA complement) to the original parent cell due to natural, accidental, or deliberate mutation. A host cell may include cells transfected or transformed in vivo with an expression vector.

As used herein, the term “introduced,” in the context of inserting a nucleic acid sequence or a polynucleotide sequence into a cell, may include transfection, transformation, or transduction and refers to the incorporation of a nucleic acid sequence or polynucleotide sequence into a eukaryotic or prokaryotic cell wherein the nucleic acid sequence or polynucleotide sequence may be incorporated into the genome of the cell (e.g., chromosome, plasmid, plastid, or mitochondrial DNA), converted into an autonomous replicon, or transiently expressed.

As used herein, the term “non-naturally occurring” when used in reference to a microbial organism or microorganism of the invention is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Non-naturally occurring microbial organisms of the disclosure can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely. Those skilled in the art will understand that the genetic alterations, including metabolic modifications exemplified herein, are described with reference to a suitable host organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

As used herein, the term “operably linked” may refer to a juxtaposition or arrangement of specified elements that allows them to perform in concert to bring about an effect. For example, a promoter may be operably linked to a coding sequence if it controls the transcription of the coding sequence.

As used herein, “1,2-propanediol” is intended to mean propylene glycol with general formula CH₃CH(OH)CH₂OH (CAS number—57-55-6).

As used herein, “1-propanol” is intended to mean n-propanol with a general formula CH₃CH₂CH₂OH (CAS number—71-23-8).

As used herein, “2-propanol” is intended to mean isopropyl alcohol with a general formula CH₃CH₃CHOH (CAS number—67-63-0).

As used herein, the term “a promoter” may refer to a regulatory sequence that is involved in binding RNA polymerase to initiate transcription of a gene. A promoter may be an inducible promoter or a constitutive promoter. An inducible promoter is a promoter that is active under environmental or developmental regulatory conditions.

As used herein, the term “a polynucleotide” or “nucleic acid sequence” may refer to a polymeric form of nucleotides of any length and any three-dimensional structure and single- or multi-stranded (e.g., single-stranded, double-stranded, triple-helical, etc.), which contain deoxyribonucleotides, ribonucleotides, and/or analogs or modified forms of deoxyribonucleotides or ribonucleotides, including modified nucleotides or bases or their analogs. Such polynucleiotides or nucleic acid sequences may encode amino acids (e.g., polypeptides or proteins such as fusion proteins). Because the genetic code is degenerate, more than one codon may be used to encode a particular amino acid, and the present disclosure encompasses polynucleotides which encode a particular amino acid sequence. Any type of modified nucleotide or nucleotide analog may be used, so long as the polynucleotide retains the desired functionality under conditions of use, including modifications that increase nuclease resistance (e.g., deoxy, 2′-O-Me, phosphorothioates, etc.). Labels may also be incorporated for purposes of detection or capture, for example, radioactive or nonradioactive labels or anchors, e.g., biotin. The term polynucleotide also includes peptide nucleic acids (PNA). Polynucleotides may be naturally occurring or non-naturally occurring. The terms polynucleotide, nucleic acid, and oligonucleotide are used herein interchangeably. Polynucleotides may contain RNA, DNA, or both, and/or modified forms and/or analogs thereof. A sequence of nucleotides may be interrupted by non-nucleotide components. One or more phosphodiester linkages may be replaced by alternative linking groups. These alternative linking groups include, but are not limited to, embodiments wherein phosphate is replaced by P(O)S (thioate), P(S)S (dithioate), (O)NR₂ (amidate), P(O)R, P(O)OR′, COCH₂ (formacetal), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. Polynucleotides may be linear or circular or comprise a combination of linear and circular portions.

As used herein, the term a “protein” or “polypeptide” may refer to a composition comprised of amino acids and recognized as a protein by those of skill in the art. The conventional one-letter or three-letter code for amino acid residues is used herein. The terms protein and polypeptide are used interchangeably herein to refer to polymers of amino acids of any length, including those comprising linked (e.g., fused) peptides/polypeptides (e.g., fusion proteins). The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, related proteins, polypeptides or peptides may encompass variant proteins, polypeptides or peptides. Variant proteins, polypeptides or peptides differ from a parent protein, polypeptide or peptide and/or from one another by a small number of amino acid residues. In some embodiments, the number of different amino acid residues is any of about 1, 2, 3, 4, 5, 10, 20, 25, 30, 35, 40, 45, or 50. In some embodiments, variants differ by about 1 to about 10 amino acids. Alternatively or additionally, variants may have a specified degree of sequence identity with a reference protein or nucleic acid, e.g., as determined using a sequence alignment tool, such as BLAST, ALIGN, and CLUSTAL (see, infra). For example, variant proteins or nucleic acid may have at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% amino acid sequence identity with a reference sequence.

As used herein, the term “recovered,” “isolated,” “purified,” and “separated” may refer to a material (e.g., a protein, peptide, nucleic acid, polynucleotide or cell) that is removed from at least one component with which it is naturally associated. For example, these terms may refer to a material which is substantially or essentially free from components which normally accompany it as found in its native state, such as, for example, an intact biological system.

As used herein, the term “recombinant” may refer to nucleic acid sequences or polynucleotides, polypeptides or proteins, and cells based thereon, that have been manipulated by man such that they are not the same as nucleic acids, polypeptides, and cells as found in nature. Recombinant may also refer to genetic material (e.g., nucleic acid sequences or polynucleotides, the polypeptides or proteins they encode, and vectors and cells comprising such nucleic acid sequences or polynucleotides) that has been modified to alter its sequence or expression characteristics, such as by mutating the coding sequence to produce an altered polypeptide, fusing the coding sequence to that of another coding sequence or gene, placing a gene under the control of a different promoter, expressing a gene in a heterologous organism, expressing a gene at decreased or elevated levels, expressing a gene conditionally or constitutively in manners different from its natural expression profile, and the like.

As used herein, the term “selective marker” or “selectable marker” may refer to a gene capable of expression in a host cell that allows for ease of selection of those hosts containing an introduced nucleic acid sequence, polynucleotide or vector. Examples of selectable markers include but are not limited to antimicrobial substances (e.g., hygromycin, bleomycin, or chloramphenicol) and/or genes that confer a metabolic advantage, such as a nutritional advantage, on the host cell.

As used herein, the term “substantially anaerobic” means that growth of the modified micororganism takes place in culture media that comprises a dissolved oxygen concentration of less than 5 ppm.

As used herein, the term “substantially similar” and “substantially identical” in the context of at least two nucleic acids, polynucleotides, proteins or polypeptides may mean that a nucleic acid, polynucleotide, protein or polypeptide comprises a sequence that has at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% sequence identity, in comparison with a reference (e.g., wild-type) nucleic acid, polynucleotide, protein or polypeptide. Sequence identity may be determined using known programs such as BLAST, ALIGN, and CLUSTAL using standard parameters. (See, e.g., Altshul et al. (1990) J. Mol. Biol. 215:403-410; Henikoff et al. (1989) Proc. Natl. Acad. Sci. 89:10915; Karin et al. (1993) Proc. Natl. Acad. Sci. 90:5873; and Higgins et al. (1988) Gene 73:237). Software for performing BLAST analyses is publicly available through the National Center for Biotechnology Information. Also, databases may be searched using FASTA (Person et al. (1988) Proc. Natl. Acad. Sci. 85:2444-2448.) In some embodiments, substantially identical polypeptides differ only by one or more conservative amino acid substitutions. In some embodiments, substantially identical polypeptides are immunologically cross-reactive. In some embodiments, substantially identical nucleic acid molecules hybridize to each other under stringent conditions (e.g., within a range of medium to high stringency).

As used herein, the term “transfection” or “transformation” may refer to the insertion of an exogenous nucleic acid or polynucleotide into a host cell. The exogenous nucleic acid or polynucleotide may be maintained as a non-integrated vector, for example, a plasmid, or alternatively, may be integrated into the host cell genome. The term transfecting or transfection is intended to encompass all conventional techniques for introducing nucleic acid or polynucleotide into host cells. Examples of transfection techniques include, but are not limited to, calcium phosphate precipitation, DEAE-dextran-mediated transfection, lipofection, electroporation, and microinjection.

As used herein, the term “transformed,” “stably transformed,” and “transgenic” may refer to a cell that has a non-native (e.g., heterologous) nucleic acid sequence or polynucleotide sequence integrated into its genome or as an episomal plasmid that is maintained through multiple generations.

As used herein, the term “vector” may refer to a polynucleotide sequence designed to introduce nucleic acids into one or more cell types. Vectors include cloning vectors, expression vectors, shuttle vectors, plasmids, phage particles, single and double stranded cassettes and the like.

As used herein, the term “wild-type,” “native,” or “naturally-occurring” proteins may refer to those proteins found in nature. The terms wild-type sequence refers to an amino acid or nucleic acid sequence that is found in nature or naturally occurring. In some embodiments, a wild-type sequence is the starting point of a protein engineering project, for example, production of variant proteins.

Unless defined otherwise herein, all technical and scientific terms used herein have the same meaning as commonly understood by one of ordinary skill in the art to which this disclosure belongs. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this disclosure. Further, it will be understood that any of the substrates disclosed in any of the pathways herein may alternatively include the anion or the cation of the substrate.

Numeric ranges provided herein are inclusive of the numbers defining the range.

Unless otherwise indicated, nucleic acids sequences are written left to right in 5′ to 3′ orientation; amino acid sequences are written left to right in amino to carboxy orientation, respectively.

While the present disclosure is capable of being embodied in various forms, the description below of several embodiments is made with the understanding that the present disclosure is to be considered as an exemplification of the disclosure, and is not intended to limit the disclosure to the specific embodiments illustrated. Headings are provided for convenience only and are not to be construed to limit the disclosure in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present disclosure.

### Modification of Microorganism

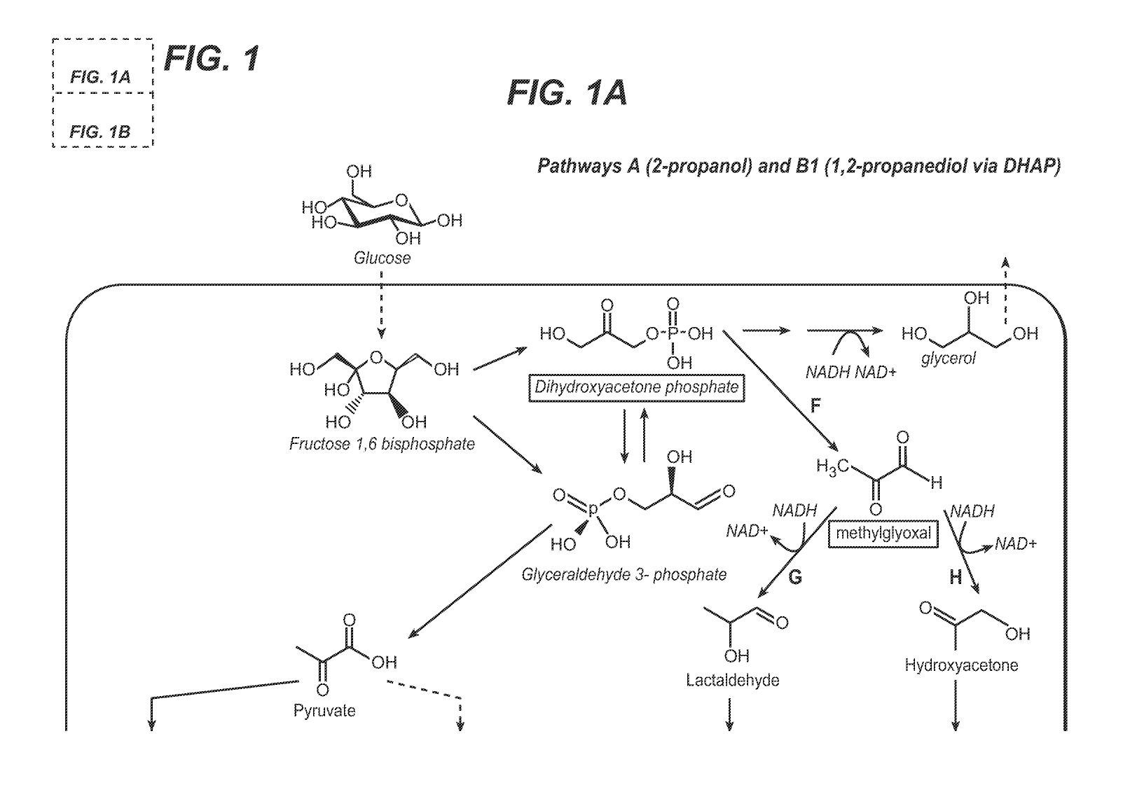

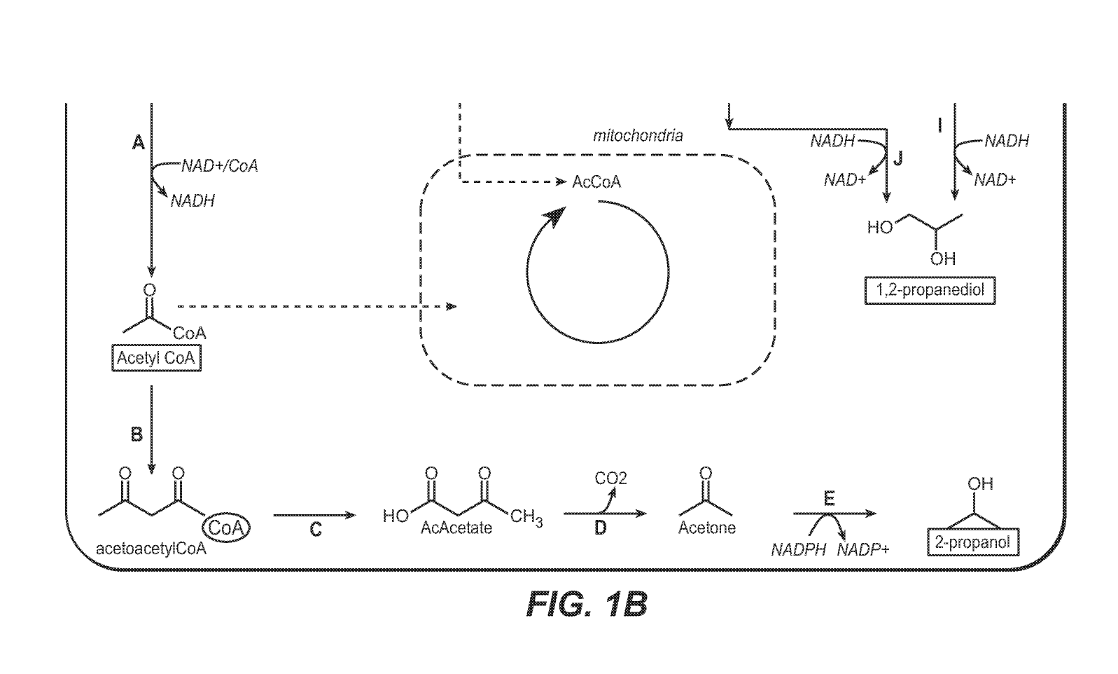

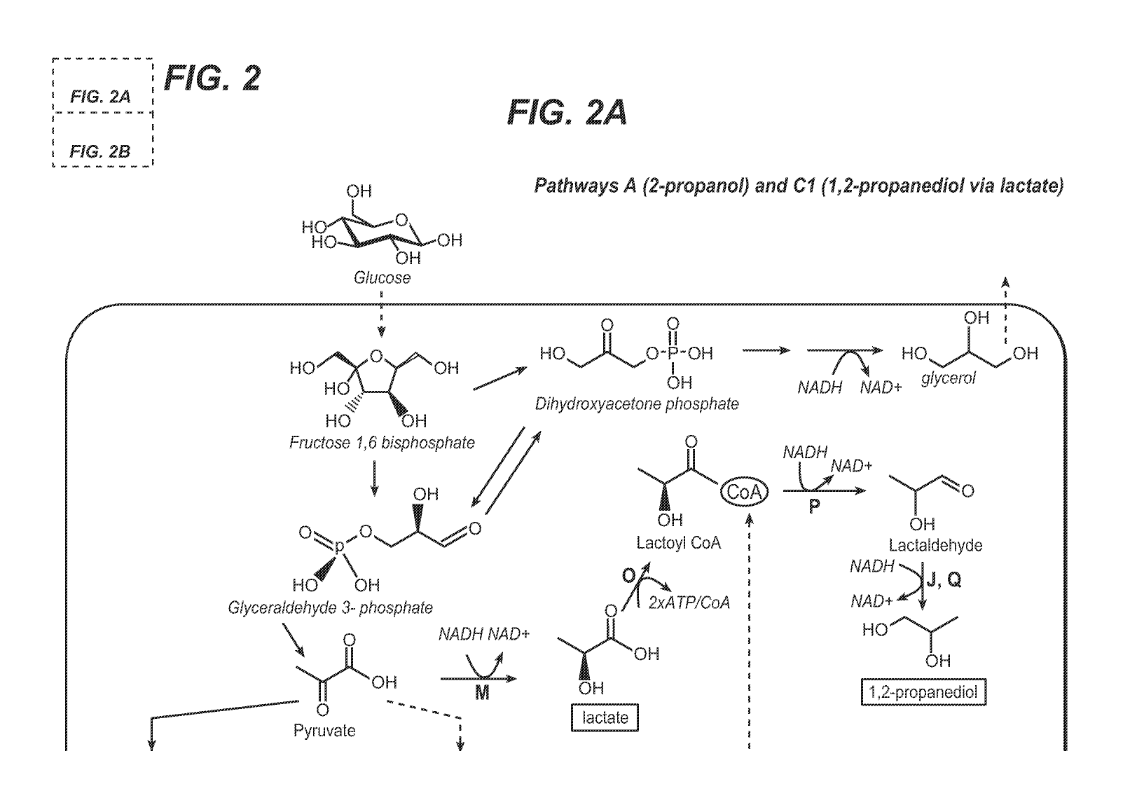

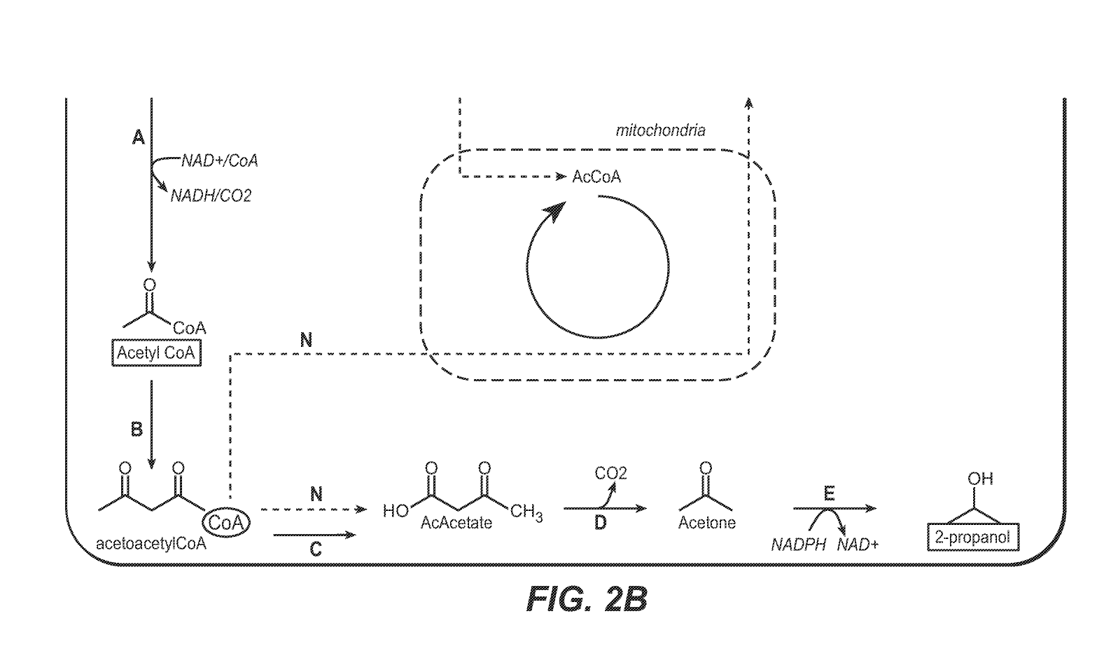

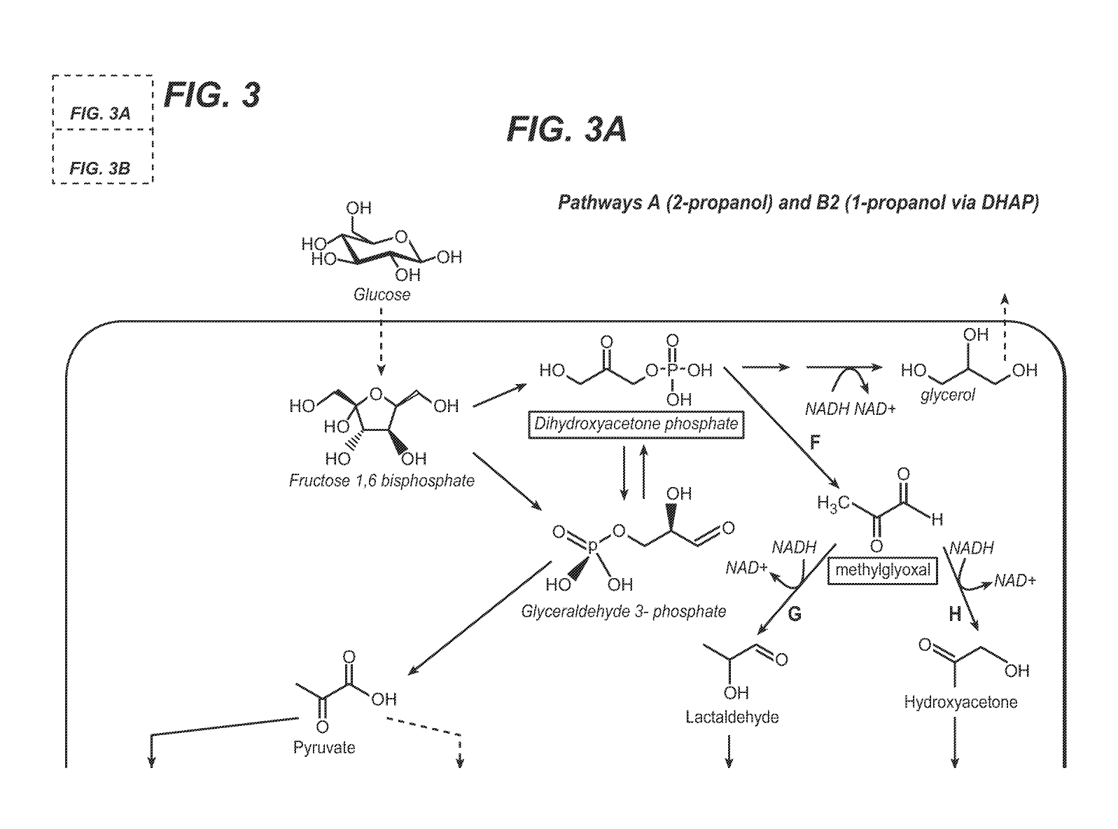

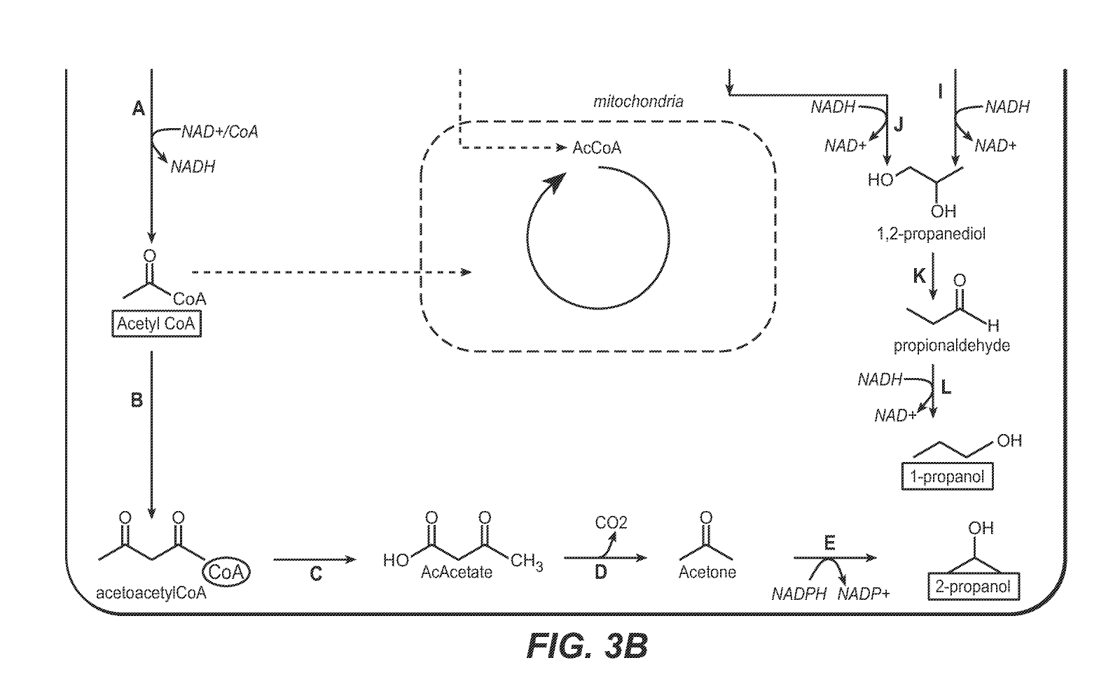

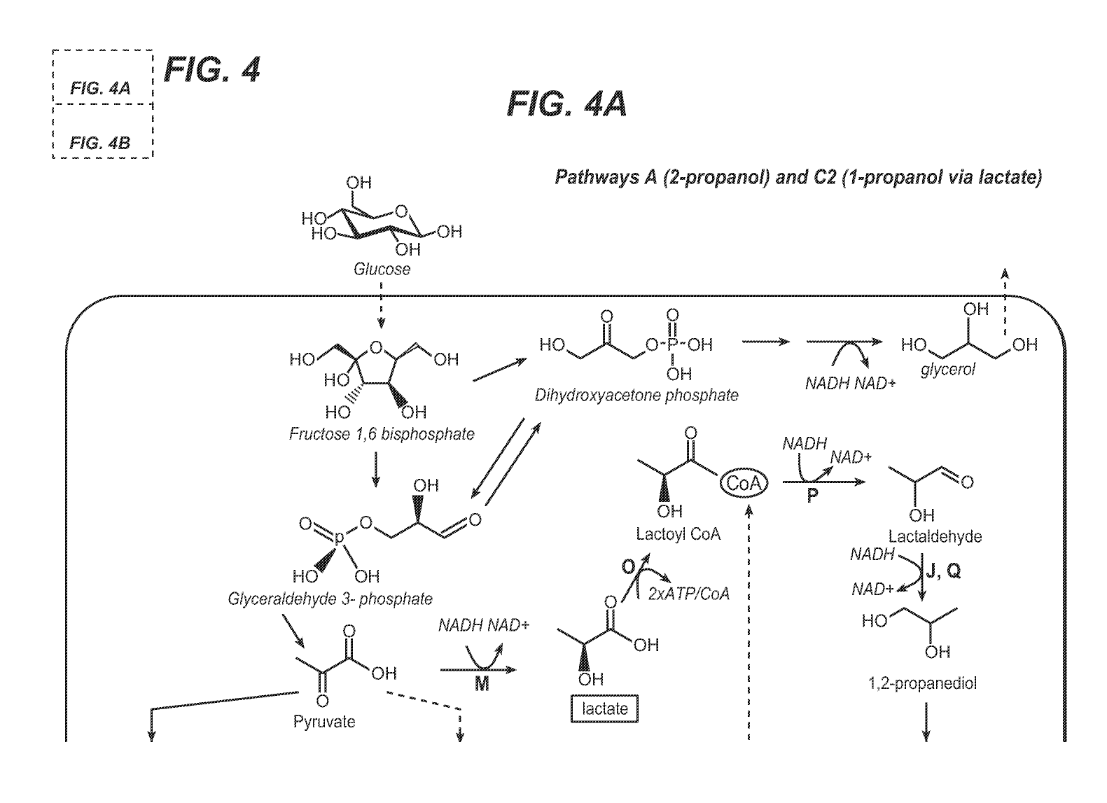

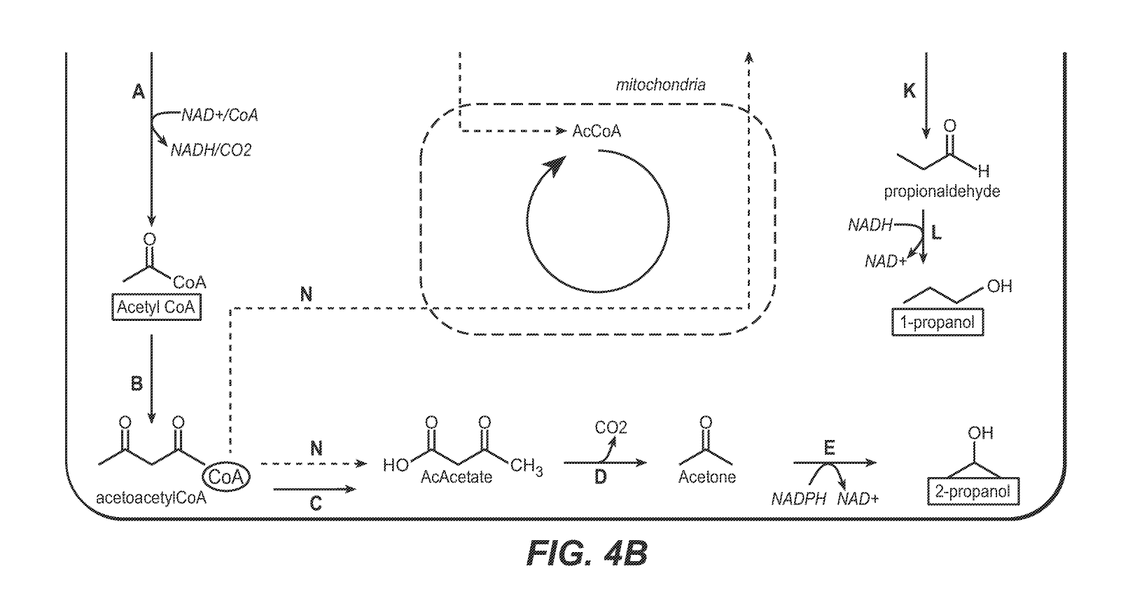

A microorganism may be modified (e.g., genetically engineered) by any method known in the art to comprise and/or express one or more polynucleotides coding for enzymes in a pathway that catalyze a conversion of a fermentable carbon source to one or more intermediates in a pathway for the co-production of 2-propanol and 1-propanol and/or 1,2-propanediol. Such enzymes may include any of those enzymes as set forth in FIGS. 1-4. For example, the microorganism may be modified to comprise one or more polynucleotides coding for enzymes that catalyze a conversion of dihydroxyacetone phosphate and/or pyruvate to 1,2-propanodiol or 1-propanol. Modified microorganisms may be referred to herein as non-naturally occuring microorganisms.

In some embodiments, the non-naturally microorganism may comprise one or more exogenous polynucleotides encoding one or more enzymes in pathways for the co-production of 2-propanol and 1-propanol and/or 1,2-propanediol from a fermentable carbon source under anaerobic or micro-anaerobic conditions.

In some embodiments, the non-naturally microorganism may comprise one or more polynucleotides coding for enzymes in a pathway that catalyzes a conversion of pyruvate to 2-propanol including, for example, one or more polynucleotides coding for enzymes in a pathway that catalyze a conversion of pyruvate to acetyl-CoA, one or more polynucleotides coding for enzymes in a pathway that catalyze a conversion of acetyl-CoA to acetoacetyl-CoA, one or more polynucleotides coding for enzymes in a pathway that catalyze a conversion of acetoacetyl-CoA to acetoacetate, one or more polynucleotides coding for enzymes in a pathway that catalyze a conversion of acetoacetate to acetone, and/or one or more polynucleotides coding for enzymes in a pathway that catalyze a conversion of acetone to 2-propanol. Enzymes catalyzing any of these conversions may include, for example, those enzymes listed in Table 1.

In some embodiments, the non-naturally occurring microorganism may comprise one or more polynucleotides coding for enzymes in a pathway that catalyzes a conversion of dihydroxyacetone-phosphate to 1,2-propanediol including, for example: one or more polynucleotides coding for enzymes in a pathway that catalyze a conversion of dihydroxyacetone-phosphate to methylglyoxal, one or more polynucleotides coding for enzymes in a pathway that catalyze a conversion of methylglyoxal to hydroxyacetone, one or more polynucleotides coding for enzymes in a pathway that catalyze a conversion of hydroxyacetone to 1,2-propanediol, one or more polynucleotides coding for enzymes in a pathway that catalyze a conversion of methylglyoxal to lactaldehyde and/or one or more polynucleotides coding for enzymes in a pathway that catalyze a conversion of lactaldehyde to 1,2-propanediol. Enzymes catalyzing any of these conversions may include, for example, those enzymes listed in Table 2.

In some embodiments, the non-naturally occurring microorganism may comprise one or more polynucleotides coding for enzymes in a pathway that catalyzes a conversion of lactate to 1,2-propanediol including, for example, one or more polynucleotides coding for enzymes in a pathway that catalyze a conversion of lactate to lactoyl-CoA, one or more polynucleotides coding for enzymes in a pathway that catalyze a conversion of lactoyl-CoA to lactaldehyde and/or one or more polynucleotides coding for enzymes in a pathway that catalyze a conversion of lactaldehyde to 1,2-propanediol. Enzymes catalyzing any of these conversions may include, for example, those enzymes listed in Table 3.

A modified microorganism as provided herein may comprise:

one or more polynucleotides coding for enzymes in a pathway that catalyzes a conversion of pyruvate to cytosolic acetyl-CoA,

one or more polynucleotides coding for enzymes in a pathway that catalyzes a conversion of acetyl-CoA to acetoacetyl-CoA,

one or more polynucleotides coding for enzymes in a pathway that catalyzes a conversion of acetoacetyl-CoA to AcAcetate,

one or more polynucleotides coding for enzymes in a pathway that catalyzes a conversion of AcAcetate to acetone,

one or more polynucleotides coding for enzymes in a pathway that catalyzes a conversion of acetone to 2-propanol,

one or more polynucleotides coding for enzymes in a pathway that catalyzes a conversion of dihydroxyacetone phosphate to methylglyoxal,

one or more polynucleotides coding for enzymes in a pathway that catalyzes a conversion of methylglyoxal to lactaldehyde,

one or more polynucleotides coding for enzymes in a pathway that catalyzes a conversion of methylglyoxal to hydroxyacetone,

one or more polynucleotides coding for enzymes in a pathway that catalyzes a conversion of hydroxyacetone to 1,2-propanediol, and/or

one or more polynucleotides coding for enzymes in a pathway that catalyzes a conversion of lactaldehyde to 1,2-propanediol.

In some embodiments, the modified microorganism has a disruption in the one or more polynucleotides that code for enzymes that decarboxylate pyruvate and associated transcription factor (e.g., pyruvate decarboxylase 1, 2, 5, and 6). In some embodiments, the modified microorganism has a disruption in each polynucleotide that codes for enzymes that decarboxylate pyruvate and associated transcription factor (e.g., pyruvate decarboxylase 1, 2, 5, and 6). In some embodiments, the modified microorganism is capable of growth on a C6 sugar as a sole carbon source under anaerobic conditions. In some embodiments, the modified microorganism has a disruption in the one or more polynucleotides that code for enzymes that decarboxylate pyruvate and associated transcription factor (e.g., pyruvate decarboxylase 1, 2, 5, and 6) and is capable of growth on a C6 sugar as a sole carbon source under anaerobic conditions. In some embodiments, the modified microorganism has a disruption in each polynucleotide that codes for enzymes that decarboxylate pyruvate and associated transcription factor (e.g., pyruvate decarboxylase 1, 2, 5, and 6) and is capable of growth on a C6 sugar as a sole carbon source under anaerobic conditions.

A modified microorganism as provided herein may comprise:

one or more polynucleotides coding for enzymes in a pathway that catalyzes a conversion of pyruvate to lactate,

one or more polynucleotides coding for enzymes in a pathway that catalyzes a conversion of lactate to lactoyl-CoA,

one or more polynucleotides coding for enzymes in a pathway that catalyzes a conversion of lactoyl-CoA to lactaldehyde,

one or more polynucleotides coding for enzymes in a pathway that catalyzes a conversion of lactaldehyde to 1,2-propanediol,

one or more polynucleotides coding for enzymes in a pathway that catalyzes a conversion of pyruvate to acetyl-CoA,

one or more polynucleotides coding for enzymes in a pathway that catalyzes a conversion of acetyl-CoA to acetoacetyl-CoA,

one or more polynucleotides coding for enzymes in a pathway that catalyzes a conversion of acetoacetyl-CoA to AcAcetate,

one or more polynucleotides coding for enzymes in a pathway that catalyzes a conversion of AcAcetate to acetone, and/or

one or more polynucleotides coding for enzymes in a pathway that catalyzes a conversion of acetone to 2-propanol.

In some embodiments, the modified microorganism has a disruption in each of the one or more polynucleotides that code for enzymes that decarboxylate pyruvate (e.g., pyruvate decarboxylase 1, 5, and 6). In some embodiments, the modified microorganism is capable of growth on a C6 sugar as a sole carbon source under anaerobic conditions. In some embodiments, the modified microorganism has a disruption in each of the one or more polynucleotides that code for enzymes that decarboxylate pyruvate (e.g., pyruvate decarboxylase 1, 5, and 6) and is capable of growth on a C6 sugar as a sole carbon source under anaerobic conditions.

A modified microorganism as provided herein may comprise:

one or more polynucleotides coding for enzymes in a pathway that catalyzes a conversion of pyruvate to cytosolic acetyl-CoA,

one or more polynucleotides coding for enzymes in a pathway that catalyzes a conversion of acetyl-CoA to acetoacetyl-CoA,

one or more polynucleotides coding for enzymes in a pathway that catalyzes a conversion of acetoacetyl-CoA to AcAcetate,

one or more polynucleotides coding for enzymes in a pathway that catalyzes a conversion of AcAcetate to acetone,

one or more polynucleotides coding for enzymes in a pathway that catalyzes a conversion of acetone to 2-propanol,

one or more polynucleotides coding for enzymes in a pathway that catalyzes a conversion of dihydroxyacetone phosphate to methylglyoxal,

one or more polynucleotides coding for enzymes in a pathway that catalyzes a conversion of methylglyoxal to lactaldehyde,

one or more polynucleotides coding for enzymes in a pathway that catalyzes a conversion of methylglyoxal to hydroxyacetone,

one or more polynucleotides coding for enzymes in a pathway that catalyzes a conversion of hydroxyacetone to 1,2-propanediol,

one or more polynucleotides coding for enzymes in a pathway that catalyzes a conversion of lactaldehyde to 1,2-propanediol,

one or more polynucleotides coding for enzymes in a pathway that catalyzes a conversion of pyruvate to lactate,

one or more polynucleotides coding for enzymes in a pathway that catalyzes a conversion of lactate to lactoyl-CoA,

one or more polynucleotides coding for enzymes in a pathway that catalyzes a conversion of lactoyl-CoA to lactaldehyde, and/or

one or more polynucleotides coding for enzymes in a pathway that catalyzes a conversion of lactaldehyde to 1,2-propanediol.

In some embodiments, the modified microorganism has a disruption in each of the one or more polynucleotides that code for enzymes that decarboxylate pyruvate (e.g., pyruvate decarboxylase 1, 5, and 6). In some embodiments, the modified microorganism is capable of growth on a C6 sugar as a sole carbon source under anaerobic conditions. In some embodiments, the modified microorganism has a disruption in each of the one or more polynucleotides that code for enzymes that decarboxylate pyruvate (e.g., pyruvate decarboxylase 1, 5, and 6) and is capable of growth on a C6 sugar as a sole carbon source under anaerobic conditions.

In some embodiments, the non-naturally occurring microorganism may comprise one or more polynucleotides coding for enzymes in a pathway that catalyzes a conversion of dihydroxyacetone-phosphate to 1-propanol including, for example: one or more polynucleotides coding for enzymes in a pathway that catalyze a conversion of dihydroxyacetone-phosphate to methylglyoxal, one or more polynucleotides coding for enzymes in a pathway that catalyze a conversion of methylglyoxal to hydroxyacetone, one or more polynucleotides coding for enzymes in a pathway that catalyze a conversion of hydroxyacetone to 1,2-propanediol, one or more polynucleotides coding for enzymes in a pathway that catalyze a conversion of methylglyoxal to lactaldehyde, one or more polynucleotides coding for enzymes in a pathway that catalyze a conversion of lactaldehyde to 1,2-propanediol, one or more polynucleotides coding for enzymes in a pathway that catalyze a conversion of 1,2-propanediol to propionaldehyde, and/or one or more polynucleotides coding for enzymes in a pathway that catalyze a conversion of propionaldehyde to 1-propanol. Enzymes catalyzing any of these conversions may include, for example, those enzymes listed in Table 2.

In some embodiments, the non-naturally occurring microorganism may comprise one or more polynucleotides coding for enzymes in a pathway that catalyzes a conversion of lactate to 1-propanol including, for example, one or more polynucleotides coding for enzymes in a pathway that catalyze a conversion of lactate to lactoyl-CoA, one or more polynucleotides coding for enzymes in a pathway that catalyze a conversion of lactoyl-CoA to lactaldehyde, one or more polynucleotides coding for enzymes in a pathway that catalyze a conversion of lactaldehyde to 1,2-propanediol, one or more polynucleotides coding for enzymes in a pathway that catalyze a conversion of 1,2-propanediol to propionaldehyde, and/or one or more polynucleotides coding for enzymes in a pathway that catalyze a conversion of propionaldehyde to 1-propanol. Enzymes catalyzing any of these conversions may include, for example, those enzymes listed in Table 3.

A modified microorganism as provided herein may comprise:

one or more polynucleotides coding for enzymes in a pathway that catalyzes a conversion of pyruvate to cytosolic acetyl-CoA,

one or more polynucleotides coding for enzymes in a pathway that catalyzes a conversion of acetyl-CoA to acetoacetyl-CoA,

one or more polynucleotides coding for enzymes in a pathway that catalyzes a conversion of acetoacetyl-CoA to AcAcetate,

one or more polynucleotides coding for enzymes in a pathway that catalyzes a conversion of AcAcetate to acetone,

one or more polynucleotides coding for enzymes in a pathway that catalyzes a conversion of acetone to 2-propanol,

one or more polynucleotides coding for enzymes in a pathway that catalyzes a conversion of dihydroxyacetone phosphate to methylglyoxal,

one or more polynucleotides coding for enzymes in a pathway that catalyzes a conversion of methylglyoxal to lactaldehyde,

one or more polynucleotides coding for enzymes in a pathway that catalyzes a conversion of methylglyoxal to hydroxyacetone,

one or more polynucleotides coding for enzymes in a pathway that catalyzes a conversion of hydroxyacetone to 1,2-propanediol,

one or more polynucleotides coding for enzymes in a pathway that catalyzes a conversion of lactaldehyde to 1,2-propanediol,

one or more polynucleotides coding for enzymes in a pathway that catalyzes a conversion of 1,2-propanediol to propionaldehyde, and/or

one or more polynucleotides coding for enzymes in a pathway that catalyzes a conversion of propionaldehyde to 1-propanol.

In some embodiments, the modified microorganism has a disruption in each of the one or more polynucleotides that code for enzymes that decarboxylate pyruvate and associated transcription factor (e.g., pyruvate decarboxylase 1, 2, 5, and 6). In some embodiments, the modified microorganism is capable of growth on a C6 sugar as a sole carbon source under anaerobic conditions. In some embodiments, the modified microorganism has a disruption in each of the one or more polynucleotides that code for enzymes that decarboxylate pyruvate and associated transcription factor (e.g., pyruvate decarboxylase 1, 2, 5, and 6) and is capable of growth on a C6 sugar as a sole carbon source under anaerobic conditions.

A modified microorganism as provided herein may comprise:

one or more polynucleotides coding for enzymes in a pathway that catalyzes a conversion of pyruvate to lactate,

one or more polynucleotides coding for enzymes in a pathway that catalyzes a conversion of lactate to lactoyl-CoA,

one or more polynucleotides coding for enzymes in a pathway that catalyzes a conversion of lactoyl-CoA to lactaldehyde,

one or more polynucleotides coding for enzymes in a pathway that catalyzes a conversion of lactaldehyde to 1,2-propanediol,

one or more polynucleotides coding for enzymes in a pathway that catalyzes a conversion of 1,2-propanediol to propionaldehyde,

one or more polynucleotides coding for enzymes in a pathway that catalyzes a conversion of propionaldehyde to 1-propanol,

one or more polynucleotides coding for enzymes in a pathway that catalyzes a conversion of pyruvate to acetyl-CoA,

one or more polynucleotides coding for enzymes in a pathway that catalyzes a conversion of acetyl-CoA to acetoacetyl-CoA,

one or more polynucleotides coding for enzymes in a pathway that catalyzes a conversion of acetoacetyl-CoA to AcAcetate,

one or more polynucleotides coding for enzymes in a pathway that catalyzes a conversion of AcAcetate to acetone, and/or

one or more polynucleotides coding for enzymes in a pathway that catalyzes a conversion of acetone to 2-propanol.

In some embodiments, the modified microorganism has a disruption in each of the one or more polynucleotides that code for enzymes that decarboxylate pyruvate (e.g., pyruvate decarboxylase 1, 5, and 6). In some embodiments, the modified microorganism is capable of growth on a C6 sugar as a sole carbon source under anaerobic conditions. In some embodiments, the modified microorganism has a disruption in each of the one or more polynucleotides that code for enzymes that decarboxylate pyruvate (e.g., pyruvate decarboxylase 1, 5, and 6) and is capable of growth on a C6 sugar as a sole carbon source under anaerobic conditions.

modified microorganism as provided herein may comprise:

one or more polynucleotides coding for enzymes in a pathway that catalyzes a conversion of pyruvate to cytosolic acetyl-CoA,

one or more polynucleotides coding for enzymes in a pathway that catalyzes a conversion of acetyl-CoA to acetoacetyl-CoA,

one or more polynucleotides coding for enzymes in a pathway that catalyzes a conversion of acetoacetyl-CoA to AcAcetate,

one or more polynucleotides coding for enzymes in a pathway that catalyzes a conversion of AcAcetate to acetone,

one or more polynucleotides coding for enzymes in a pathway that catalyzes a conversion of acetone to 2-propanol,

one or more polynucleotides coding for enzymes in a pathway that catalyzes a conversion of dihydroxyacetone phosphate to methylglyoxal,

one or more polynucleotides coding for enzymes in a pathway that catalyzes a conversion of methylglyoxal to lactaldehyde,

one or more polynucleotides coding for enzymes in a pathway that catalyzes a conversion of methylglyoxal to hydroxyacetone,

one or more polynucleotides coding for enzymes in a pathway that catalyzes a conversion of hydroxyacetone to 1,2-propanediol,

one or more polynucleotides coding for enzymes in a pathway that catalyzes a conversion of lactaldehyde to 1,2-propanediol,

one or more polynucleotides coding for enzymes in a pathway that catalyzes a conversion of 1,2-propanediol to propionaldehyde,

one or more polynucleotides coding for enzymes in a pathway that catalyzes a conversion of propionaldehyde to 1-propanol.

one or more polynucleotides coding for enzymes in a pathway that catalyzes a conversion of pyruvate to lactate,

one or more polynucleotides coding for enzymes in a pathway that catalyzes a conversion of lactate to lactoyl-CoA,

one or more polynucleotides coding for enzymes in a pathway that catalyzes a conversion of lactoyl-CoA to lactaldehyde,

one or more polynucleotides coding for enzymes in a pathway that catalyzes a conversion of lactaldehyde to 1,2-propanediol,

one or more polynucleotides coding for enzymes in a pathway that catalyzes a conversion of 1,2-propanediol to propionaldehyde, and/or

one or more polynucleotides coding for enzymes in a pathway that catalyzes a conversion of propionaldehyde to 1-propanol.

In some embodiments, the modified microorganism has a disruption in each of the one or more polynucleotides that code for enzymes that decarboxylate pyruvate (e.g., pyruvate decarboxylase 1, 5, and 6). In some embodiments, the modified microorganism is capable of growth on a C6 sugar as a sole carbon source under anaerobic conditions. In some embodiments, the modified microorganism has a disruption in each of the one or more polynucleotides that code for enzymes that decarboxylate pyruvate (e.g., pyruvate decarboxylase 1, 5, and 6) and is capable of growth on a C6 sugar as a sole carbon source under anaerobic conditions.

Exemplary enzymes that convert a fermentable carbon source such as glucose to 1,2-propanediol (Pathway B1) and/or 2-propanol (Pathway A) and 1-propanol (Pathways B2 and C2) and/or 2-propanol (Pathway A) including, enzyme substrates, and enzyme reaction products associated with the conversions are presented in Tables 1 to 5 below. The enzyme reference identifier listed in Tables 1 to 4 correlates with the enzyme numbering used in FIGS. 1-4, which schematically represents the enzymatic conversion of a fermentable carbon source such as glucose to dihydroxyacetone phosphate or lactate and pyruvate. Dihydroxyacetone phosphate or lactate and pyruvate may be further converted to 1,2-propanediol,1-propanol and/or 2-propanol, using any combination of one or more enzymes provided in Tables 1 to 5 below including, all of those enzymes as provided in Tables 1 to 5 below.

TABLE 1
Pathway A (2-propanol from pyruvate)

| Enzyme | | EC | |
|---|---|---|---|
| No. | Enzyme name | Number | Reaction |
| A1. | Formate-C | 2.3.1.54 | Pyruvate + CoA → |
| | acetyltransferase | 1.97.1.4 | Acetyl-CoA + formate |
| | Formate-C | | |
| | acetyltransferase | | |
| | activating enzyme | | |
| A2. | Pyruvate dehydrogenase | 1.2.4.1 | Pyruvate + CoA + NAD⁺ → |
| | | 2.3.1.12 | Acetyl-CoA + CO₂ + NADH |
| | | 1.8.1.4 | |
| B. | Thiolase | 2.3.1.9 | 2 acetyl-CoA → acetoacetyl-CoA + |
| | | | CoA |
| C. | Acetoacetyl-CoA | 2.8.3.8 | acetoacetyl-Coa + acetate → |
| | acetyltransferase | | acetoacetate + acetyl-CoA |
| D. | Acetatoacetate | 4.1.1.4 | acetoacetate → acetone + CO₂ |
| | decarboxylase | | |
| E. | Secondary alcohol | 1.1.1.2 | acetone + NAD(P)H→ |
| | dehydrogenase | | 2-propanol + NAD(P)+ |

TABLE 2
Pathway B1 (1,2-propanediol from Dihydroxyacetone phosphate

| Enzyme | | EC | |
|---|---|---|---|
| No. | Enzyme name | Number | Reaction |
| F1. | methylglyoxal synthase | 4.2.3.3 | dihydroxyacetone phosphate → |
| | | | methylglyoxal |
| F2. | methylglyoxal synthase, | 4.2.3.3 | dihydroxyacetone phosphate → |
| | phosphate insensitive | | methylglyoxal |
| G. | Methylglyoxal reductase | 1.1.1.— | Methylglyoxal → lactaldehyde |
| H. | Methylglyoxal reductase | 1.1.1.78 | methylglyoxal → hydroxyacetone |
| I. | methylglyoxal reductase | 1.1.1.— | Hydroxyacetone + NAD(P)H + H⁺ →1,2- |
| | [multifunctional] | | propanediol + NAD(P)⁺ |
| J. | methylglyoxal reductase | 1.1.1.— | Lactaldehyde + NAD(P)H + H⁺ →1,2- |
| | [multifunctional] | | propanediol + NAD(P)⁺ |

TABLE 3
Pathway C1 (1,2-propanediol from lactate)

| Enzyme | | EC | |
|---|---|---|---|
| No. | Enzyme name | Number | Reaction |
| M1. | D-Lactate dehydrogenase | 1.1.1.28 | Pyruvate + NAD(P)H + H⁺ −>D- |
| | | | Lactate + NAD(P)⁺ |
| M2. | L-Lactate dehydrogenase | 1.1.1.27 | Pyruvate + NAD(P)H + H⁺ −>L- |
| | | | Lactate + NAD(P)⁺ |
| N. | propionate CoA- | 2.8.3.1 | Lactate + Acetyl-CoA −> |
| | transferase* | | Lactoyl-CoA + Acetic acid |
| O. | Lactoyl-CoA synthase | 2.3.3.— | Lactate + CoA + ATP → |
| | | | lactoyl-CoA + AMP |
| P. | 1,2-propanediol | 1.2.1.— | Lactoyl-CoA + NAD(P)H + |
| | oxidoreductase | | H⁺ −> Lactaldehyde + |
| | | | NAD(P)⁺ |
| Q. | Lactaldehyde reductase | 1.1.1.77 | L-Lactaldehyde + NAD(P)H + H⁺ −>L- |
| | | | 1,2-propanediol + NAD(P)⁺ |
| J. | methylglyoxal reductase | 1.1.1.— | Lactaldehyde + NAD(P)H + H⁺ −>1,2- |
| | [multifunctional] | | propanediol + NAD(P)⁺ |

*enzyme with homologous function but altered substrate specificity is required/preferred

TABLE 4
Pathway B2 (1-propanol from Dihydroxyacetone phosphate

| Enzyme | | EC | |
|---|---|---|---|
| No. | Enzyme name | Number | Reaction |
| F1. | methylglyoxal synthase | 4.2.3.3 | dihydroxyacetone phosphate → |
| | | | methylglyoxal |
| F2. | methylglyoxal synthase, | 4.2.3.3 | dihydroxyacetone phosphate → |
| | phosphate insensitive | | methylglyoxal |
| G. | Methylglyoxal reductase | 1.1.1.— | Methylglyoxal → lactaldehyde |
| H. | Methylglyoxal reductase | 1.1.1.78 | methylglyoxal → hydroxyacetone |
| I. | methylglyoxal reductase | 1.1.1.— | Hydroxyacetone + NAD(P)H + H⁺ →1,2- |
| | [multifunctional] | | propanediol + NAD(P)⁺ |
| J. | methylglyoxal reductase | 1.1.1.— | Lactaldehyde + NAD(P)H + H⁺ →R/S |
| | [multifunctional] | | 1,2-propanediol + NAD(P)⁺ |
| K. | 1,2 propanediol dehydratase | 4.2.1.30 | R/S 1,2 propanediol → |
| | | | proprionaldehyde |
| L. | 1-propanol dehydrogenase | 1.1.1.— | proprionaldehyde + NADH → |
| | | | propanol + NAD+ |

TABLE 5
Pathway C2 (1-propanol from lactate)

| Enzyme | | EC | |
|---|---|---|---|
| No. | Enzyme name | Number | Reaction |
| M1. | D-Lactate dehydrogenase | 1.1.1.28 | Pyruvate + NAD(P)H + H⁺ →D- |
| | | | Lactate + NAD(P)⁺ |
| M2. | L-Lactate dehydrogenase | 1.1.1.27 | Pyruvate + NAD(P)H + H⁺ →L- |
| | | | Lactate + NAD(P)⁺ |
| N. | propionate CoA- | 2.8.3.1 | Lactate + Acetyl-CoA → |
| | transferase* | | Lactoyl-CoA + Acetic acid |
| O. | Lactoyl-CoA synthase | 2.3.3.— | Lactate + CoA + ATP → |
| | | | lactoyl-CoA + AMP |
| P. | 1,2-propanediol | 1.2.1.— | Lactoyl-CoA + NAD(P)H + |
| | oxidoreductase | | H⁺ →Lactaldehyde + |
| | | | NAD(P)⁺ |
| Q. | Lactaldehyde reductase | 1.1.1.77 | Lactaldehyde + NAD(P)H + H⁺ →1,2- |
| | | | propanediol + NAD(P)⁺ |
| J. | methylglyoxal reductase | 1.1.1.— | Lactaldehyde + NAD(P)H + H⁺ →R/S |
| | [multifunctional] | | 1,2-propanediol + NAD(P)⁺ |
| K. | 1,2 propanediol | 4.2.1.28 | R/S 1,2 propanediol → |
| | dehydratase | | proprionaldehyde |
| L. | 1-propanol dehydrogenase | 1.1.1.— | proprionaldehyde + NADH → |
| | | | 1-propanol + NAD+ |

*enzyme with homologous function but altered substrate specificity is required/preferred

The microorganism may be an archea, bacteria, or eukaryote. In some embodiments, the bacteria is a Propionibacterium, Propionispira, Clostridium, Bacillus, Escherichia, Pelobacter, or Lactobacillus including, for example, Pelobacter propionicus, Clostridium propionicum, Clostridium acetobutylicum, Lactobacillus, Propionibacterium acidipropionici or Propionibacterium freudenreichii. In some embodiments, the eukaryote is a yeast, filamentous fungi, protozoa, or algae. In some embodiments, the yeast is Saccharomyces cerevisiae, Kluyveromyces lactis or Pichia pastoris.

In some embodiments, the microorganism is additionally modified to comprise one or more tolerance mechanisms including, for example, tolerance to a produced molecule (i.e., methylglyoxal, 1-propanol, or 2-propanol), and/or organic solvents. A microorganism modified to comprise such a tolerance mechanism may provide a means to increase titers of fermentations and/or may control contamination in an industrial scale process.

In some embodiments, the disclosure contemplates the modification (e.g., engineering) of one or more of the enzymes provided herein. Such modification may be performed to redesign the substrate specificity of the enzyme and/or to modify (e.g., reduce) its activity against others substrates in order to increase its selectivity for a given substrate. Additionally or alternatively, one or more enzymes as provided herein may be engineered to alter (e.g., enhance including, for example, increase its catalytic activity or its substrate specificity) one or more of its properties, including acceptance of different co-factors such as NADH instead of NADPH.

In some embodiments, sequence alignment and comparative modeling of proteins may be used to alter one or more of the enzymes disclosed herein. Homology modeling or comparative modeling refers to building an atomic-resolution model of the desired protein from its primary amino acid sequence and an experimental three-dimensional structure of a similar protein. This model may allow for the enzyme substrate binding site to be defined, and the identification of specific amino acid positions that may be replaced to other natural amino acid in order to redesign its substrate specificity.

Variants or sequences having substantial identity or homology with the polynucleotides encoding enzymes as disclosed herein may be utilized in the practice of the disclosure. Such sequences can be referred to as variants or modified sequences. That is, a polynucleotide sequence may be modified yet still retain the ability to encode a polypeptide exhibiting the desired activity. Such variants or modified sequences are thus equivalents in the sense that they retain their intended function. Generally, the variant or modified sequence may comprise at least about 40%-60%, preferably about 60%-80%, more preferably about 80%-90%, and even more preferably about 90%-95% sequence identity with the native sequence.

One example of such a variant is described in SEQ ID NO: 3 wherein an E51D Glu-Asp mutation that renders the coded acetoacetyl-CoA transferase into a acetoacetyl-CoA hydrolase. Further modifications to SEQ ID NO: 3 through rational and/or random approaches may be further performed to improve hydrolase activity.

In some embodiments, a microorganism may be modified to express including, for example, overexpress, one or more enzymes as provided herein. The microorganism may be modified by genetic engineering techniques (i.e., recombinant technology), classical microbiological techniques, or a combination of such techniques and can also include naturally occurring genetic variants to produce a genetically modified microorganism. Some of such techniques are generally disclosed, for example, in Sambrook et al., 1989, Molecular Cloning: A Laboratory Manual, Cold Spring Harbor Labs Press; and Selifonova et al. (2001) Appl. Environ. Microbiol. 67(8):3645).

A genetically modified microorganism may include a microorganism in which a polynucleotide has been inserted, deleted or modified (i.e., mutated; e.g., by insertion, deletion, substitution, and/or inversion of nucleotides), in such a manner that such modifications provide the desired effect of expression (e.g., over-expression) of one or more enzymes as provided herein within the microorganism. Genetic modifications which result in an increase in gene expression or function can be referred to as amplification, overproduction, overexpression, activation, enhancement, addition, or up-regulation of a gene. Addition of cloned genes to increase gene expression can include maintaining the cloned gene(s) on replicating plasmids or integrating the cloned gene(s) into the genome of the production organism. Furthermore, increasing the expression of desired cloned genes can include operatively linking the cloned gene(s) to native or heterologous transcriptional control elements.

Where desired, the expression of one or more of the genes provided herein are under the control of a regulatory sequence that controls directly or indirectly the expression of the gene in a time-dependent fashion during a fermentation reaction.

In some embodiments, a microorganism is transformed or transfected with a genetic vehicle such as, an expression vector comprising an exogenous polynucleotide sequence coding for the enzymes provided herein.

Polynucleotide constructs prepared for introduction into a prokaryotic or eukaryotic host may typically, but not always, comprise a replication system (i.e. vector) recognized by the host, including the intended polynucleotide fragment encoding the desired polypeptide, and may preferably, but not necessarily, also include transcription and translational initiation regulatory sequences operably linked to the polypeptide-encoding segment. Expression systems (expression vectors) may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, mRNA stabilizing sequences, nucleotide sequences homologous to host chromosomal DNA, and/or a multiple cloning site. Signal peptides may also be included where appropriate, preferably from secreted polypeptides of the same or related species, which allow the protein to cross and/or lodge in cell membranes or be secreted from the cell.

The vectors can be constructed using standard methods (see, e.g., Sambrook et al., Molecular Biology: A Laboratory Manual, Cold Spring Harbor, N.Y. 1989; and Ausubel, et al., Current Protocols in Molecular Biology, Greene Publishing, Co. N.Y, 1995).

The manipulation of polynucleotides of the present disclosure including polynucleotides coding for one or more of the enzymes disclosed herein is typically carried out in recombinant vectors. Numerous vectors are publicly available, including bacterial plasmids, bacteriophage, artificial chromosomes, episomal vectors and gene expression vectors, which can all be employed. A vector of use according to the disclosure may be selected to accommodate a protein coding sequence of a desired size. A suitable host cell is transformed with the vector after in vitro cloning manipulations. Host cells may be prokaryotic, such as any of a number of bacterial strains, or may be eukaryotic, such as yeast or other fungal cells, insect or amphibian cells, or mammalian cells including, for example, rodent, simian or human cells. Each vector contains various functional components, which generally include a cloning site, an origin of replication and at least one selectable marker gene. If given vector is an expression vector, it additionally possesses one or more of the following: enhancer element, promoter, transcription termination and signal sequences, each positioned in the vicinity of the cloning site, such that they are operatively linked to the gene encoding a polypeptide repertoire member according to the disclosure.

Vectors, including cloning and expression vectors, may contain nucleic acid sequences that enable the vector to replicate in one or more selected host cells. For example, the sequence may be one that enables the vector to replicate independently of the host chromosomal DNA and may include origins of replication or autonomously replicating sequences. Such sequences are well known for a variety of bacteria, yeast and viruses. For example, the origin of replication from the plasmid pBR322 is suitable for most Gram-negative bacteria, the 2 micron plasmid origin is suitable for yeast, and various viral origins (e.g. SV 40, adenovirus) are useful for cloning vectors in mammalian cells. Generally, the origin of replication is not needed for mammalian expression vectors unless these are used in mammalian cells able to replicate high levels of DNA, such as COS cells.

A cloning or expression vector may contain a selection gene also referred to as a selectable marker. This gene encodes a protein necessary for the survival or growth of transformed host cells grown in a selective culture medium. Host cells not transformed with the vector containing the selection gene will therefore not survive in the culture medium. Typical selection genes encode proteins that confer resistance to antibiotics and other toxins, e.g. ampicillin, neomycin, methotrexate, hygromycin, thiostrepton, apramycin or tetracycline, complement auxotrophic deficiencies, or supply critical nutrients not available in the growth media.

The replication of vectors may be performed in E. coli (e.g., strain TB1 or TG1, DH5α, DH10β, JM110). An E. coli-selectable marker, for example, the β-lactamase gene that confers resistance to the antibiotic ampicillin, may be of use. These selectable markers can be obtained from E. coli plasmids, such as pBR322 or a pUC plasmid such as pUC18 or pUC19, or pUC119.

Expression vectors may contain a promoter that is recognized by the host organism. The promoter may be operably linked to a coding sequence of interest. Such a promoter may be inducible or constitutive. Polynucleotides are operably linked when the polynucleotides are in a relationship permitting them to function in their intended manner.

Promoters suitable for use with prokaryotic hosts may include, for example, the a-lactamase and lactose promoter systems, alkaline phosphatase, the tryptophan (trp) promoter system, the erythromycin promoter, apramycin promoter, hygromycin promoter, methylenomycin promoter and hybrid promoters such as the tac promoter. Moreover, host constitutive or inducible promoters may be used. Promoters for use in bacterial systems will also generally contain a Shine-Dalgarno sequence operably linked to the coding sequence.

Viral promoters obtained from the genomes of viruses include promoters from polyoma virus, fowlpox virus, adenovirus (e.g., Adenovirus 2 or 5), herpes simplex virus (thymidine kinase promoter), bovine papilloma virus, avian sarcoma virus, cytomegalovirus, a retrovirus (e.g., MoMLV, or RSV LTR), Hepatiti B virus, Myeloproliferative sarcoma virus promoter (MPSV), VISNA, and Simian Virus 40 (SV40). Heterologous mammalian promoters include, e.g., the actin promoter, immunoglobulin promoter, heat-shock protein promoters.

The early and late promoters of the SV40 virus are conveniently obtained as a restriction fragment that also contains the SV40 viral origin of replication (see, e.g., Fiers et al., Nature, 273:113 (1978); Mulligan and Berg, Science, 209:1422-1427 (1980); and Pavlakis et al., Proc. Natl. Acad. Sci. USA, 78:7398-7402 (1981)). The immediate early promoter of the human cytomegalovirus (CMV) is conveniently obtained as a Hind III E restriction fragment (see, e.g., Greenaway et al., Gene, 18:355-360 (1982)). A broad host range promoter, such as the SV40 early promoter or the Rous sarcoma virus LTR, is suitable for use in the present expression vectors.

Generally, a strong promoter may be employed to provide for high level transcription and expression of the desired product. Among the eukaryotic promoters that have been identified as strong promoters for high-level expression are the SV40 early promoter, adenovirus major late promoter, mouse metallothionein-I promoter, Rous sarcoma virus long terminal repeat, and human cytomegalovirus immediate early promoter (CMV or CMV IE). In an embodiment, the promoter is a SV40 or a CMV early promoter.

The promoters employed may be constitutive or regulatable, e.g., inducible. Exemplary inducible promoters include jun, fos and metallothionein and heat shock promoters. One or both promoters of the transcription units can be an inducible promoter. In an embodiment, the GFP is expressed from a constitutive promoter while an inducible promoter drives transcription of the gene coding for one or more enzymes as disclosed herein and/or the amplifiable selectable marker.

The transcriptional regulatory region in higher eukaryotes may comprise an enhancer sequence. Many enhancer sequences from mammalian genes are known e.g., from globin, elastase, albumin, α-fetoprotein and insulin genes. A suitable enhancer is an enhancer from a eukaryotic cell virus. Examples include the SV40 enhancer on the late side of the replication origin (bp 100-270), the enhancer of the cytomegalovirus immediate early promoter (Boshart et al. Cell 41:521 (1985)), the polyoma enhancer on the late side of the replication origin, and adenovirus enhancers (see also, e.g., Yaniv, Nature, 297:17-18 (1982) on enhancing elements for activation of eukaryotic promoters). The enhancer sequences may be introduced into the vector at a position 5′ or 3′ to the gene of interest, but is preferably located at a site 5′ to the promoter.

Yeast and mammalian expression vectors may contain prokaryotic sequences that facilitate the propagation of the vector in bacteria. Therefore, the vector may have other components such as an origin of replication (e.g., a nucleic acid sequence that enables the vector to replicate in one or more selected host cells), antibiotic resistance genes for selection in bacteria, and/or an amber stop codon which can permit translation to read through the codon. Additional eukaryotic selectable gene(s) may be incorporated. Generally, in cloning vectors the origin of replication is one that enables the vector to replicate independently of the host chromosomal DNA, and includes origins of replication or autonomously replicating sequences. Such sequences are well known, e.g., the ColE1 origin of replication in bacteria. Various viral origins (e.g., SV40, polyoma, adenovirus, VSV or BPV) are useful for cloning vectors in mammalian cells. Generally, a eukaryotic replicon is not needed for expression in mammalian cells unless extrachromosomal (episomal) replication is intended (e.g., the SV40 origin may typically be used only because it contains the early promoter).

To facilitate insertion and expression of different genes coding for the enzymes as disclosed herein from the constructs and expression vectors, the constructs may be designed with at least one cloning site for insertion of any gene coding for any enzyme disclosed herein. The cloning site may be a multiple cloning site, e.g., containing multiple restriction sites.

The plasmids may be propagated in bacterial host cells to prepare DNA stocks for subcloning steps or for introduction into eukaryotic host cells. Transfection of eukaryotic host cells can be any performed by any method well known in the art. Transfection methods include lipofection, electroporation, calcium phosphate co-precipitation, rubidium chloride or polycation mediated transfection, protoplast fusion and microinjection. Preferably, the transfection is a stable transfection. The transfection method that provides optimal transfection frequency and expression of the construct in the particular host cell line and type, is favored. Suitable methods can be determined by routine procedures. For stable transfectants, the constructs are integrated so as to be stably maintained within the host chromosome.

Vectors may be introduced to selected host cells by any of a number of suitable methods known to those skilled in the art. For example, vector constructs may be introduced to appropriate cells by any of a number of transformation methods for plasmid vectors. For example, standard calcium-chloride-mediated bacterial transformation is still commonly used to introduce naked DNA to bacteria (see, e.g., Sambrook et al., 1989, Molecular Cloning, A Laboratory Manual, Cold Spring Harbor Laboratory Press, Cold Spring Harbor, N.Y.), but electroporation and conjugation may also be used (see, e.g., Ausubel et al., 1988, Current Protocols in Molecular Biology, John Wiley & Sons, Inc., NY, N.Y.).

For the introduction of vector constructs to yeast or other fungal cells, chemical transformation methods may be used (e.g., Rose et al., 1990, Methods in Yeast Genetics, Cold Spring Harbor Laboratory Press, Cold Spring Harbor, N.Y.). Transformed cells may be isolated on selective media appropriate to the selectable marker used. Alternatively, or in addition, plates or filters lifted from plates may be scanned for GFP fluorescence to identify transformed clones.

For the introduction of vectors comprising differentially expressed sequences to mammalian cells, the method used may depend upon the form of the vector. Plasmid vectors may be introduced by any of a number of transfection methods, including, for example, lipid-mediated transfection (“lipofection”), DEAE-dextran-mediated transfection, electroporation or calcium phosphate precipitation (see, e.g., Ausubel et al., 1988, Current Protocols in Molecular Biology, John Wiley & Sons, Inc., NY, N.Y.).

Lipofection reagents and methods suitable for transient transfection of a wide variety of transformed and non-transformed or primary cells are widely available, making lipofection an attractive method of introducing constructs to eukaryotic, and particularly mammalian cells in culture. For example, LipofectAMINE™ (Life Technologies) or LipoTaxi™ (Stratagene) kits are available. Other companies offering reagents and methods for lipofection include Bio-Rad Laboratories, CLONTECH, Glen Research, InVitrogen, JBL Scientific, MBI Fermentas, PanVera, Promega, Quantum Biotechnologies, Sigma-Aldrich, and Wako Chemicals USA.

The host cell may be capable of expressing the construct encoding the desired protein, processing the protein and transporting a secreted protein to the cell surface for secretion. Processing includes co- and post-translational modification such as leader peptide cleavage, GPI attachment, glycosylation, ubiquitination, and disulfide bond formation. Immortalized host cell cultures amenable to transfection and in vitro cell culture and of the kind typically employed in genetic engineering are preferred. Examples of useful mammalian host cell lines are monkey kidney CV1 line transformed by SV40 (CO 7, ATCC CRL 1651); human embryonic kidney line (293 or 293 derivatives adapted for growth in suspension culture, Graham et al., J. Gen Virol., 36:59 (1977); baby hamster kidney cells (BHK, ATCC CCL 10); DHFR-Chinese hamster ovary cells (ATCC CRL-9096); dp12.CHO cells, a derivative of CHO/DHFR-(EP 307,247 published 15 Mar. 1989); mouse sertoli cells (TM4, Mather, Biol. Reprod., 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 (1982)); PEER human acute lymphoblastic cell line (Ravid et al. Int. J. Cancer 25:705-710 (1980)); MRC 5 cells; FS4 cells; human hepatoma line (Hep G2), human HT1080 cells, KB cells, JW-2 cells, Detroit 6 cells, NIH-3T3 cells, hybridoma and myeloma cells. Embryonic cells used for generating transgenic animals are also suitable (e.g., zygotes and embryonic stem cells).

Suitable host cells for cloning or expressing polynucleotides (e.g., DNA) in vectors may include, for example, prokaryote, yeast, or higher eukaryote cells. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41 P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), E. coli JM110 (ATCC 47,013) and E. coli W3110 (ATCC 27,325) are suitable.

In addition to prokaryotes, eukaryotic microbes such as filamentous fungi or yeast may be suitable cloning or expression hosts for vectors comprising polynucleotides coding for one or more enzymes. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastors (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

When the enzyme is glycosylated, suitable host cells for expression may be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori (silk moth) have been identified. A variety of viral strains for transfection are publicly available, e.g., the L-1 variant of Autographa califomica NPV and the Bm-5 strain of Bombyx mori NPV, and such viruses may be used as the virus herein according to the present disclosure, particularly for transfection of Spodoptera frugiperda cells.

Plant cell cultures of cotton, corn, potato, soybean, petunia, tomato, tobacco, lemna, and other plant cells can also be utilized as host cells.

Examples of useful mammalian host cells are Chinese hamster ovary cells, including CHOK1 cells (ATCC CCL61), DXB-11, DG-44, and Chinese hamster ovary cells/-DHFR (CHO, Urlaub et al., Proc. Natl. Acad. Sci. USA 77: 4216 (1980)); monkey kidney CV1 line transformed by SV40 (CO 7, ATCC CRL 1651); human embryonic kidney line (293 or 293 cells subcloned for growth in suspension culture, (Graham et al., J. Gen Virol. 36: 59, 1977); baby hamster kidney cells (BHK, ATCC CCL 10); mouse sertoli cells (TM4, Mather, (Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y Acad. Sci. 383: 44-68 (1982)); MRC 5 cells; FS4 cells; and a human hepatoma line (Hep G2).

Host cells are transformed or transfected with the above-described expression or cloning vectors for production of one or more enzymes as disclosed herein or with polynucleotides coding for one or more enzymes as disclosed herein and cultured in conventional nutrient media modified as appropriate for inducing promoters, selecting transformants, or amplifying the genes encoding the desired sequences.

Host cells containing desired nucleic acid sequences coding for the disclosed enzymes may be cultured in a variety of media. Commercially available media such as Ham's F10 (Sigma), Minimal Essential Medium ((MEM), Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44, (1979); Barnes et al., Anal. Biochem. 102: 255 (1980); U.S. Pat. Nos. 4,767,704; 4,657,866; 4,927,762; 4,560,655; or 5,122,469; WO90103430; WO 87/00195; or U.S. Pat. Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### Polynucleotides and Encoded Enzymes

Any known polynucleotide (e.g., gene) that codes for an enzyme or variant thereof that is capable of catalyzing an enzymatic conversion including, for example, an enzyme as set forth in any one of Tables 1-5 or FIGS. 1-4, is contemplated for use by the present disclosure. Such polynucleotides may be modified (e.g., genetically engineered) to modulate (e.g., increase or decrease) the substrate specificity of an encoded enzyme, or the polynucleotides may be modified to change the substrate specificity of the encoded enzyme (e.g., a polynucleotide that codes for an enzyme with specificity for a substrate may be modified such that the enzyme has specificity for an alternative substrate). Preferred microorganisms may comprise polynucleotides coding for one or more of the enzymes as set forth in Tables 1-105 and FIGS. 1-4.

Enzymes for catalyzing the conversions set forth in pathways A, B1, B2, C1, and C2 of Tables 1-5 and FIGS. 1-4 are categorized in Table 6 below. Enzyme numbers presented in Table 6 that are followed by a numeral, e.g., A1 or A2, represent alternative enzymes that can catalyze a particular conversion and may be generally referred to throughout this disclosure and figures by the first letter that precedes the numeral, e.g., A.

TABLE 6
Exemplary Gene Identifier (GI) numbers and/or Uniprot numbers

| Path- | | Enzyme | EC | | | Uniprot ID | SEQ ID NO. | SEQ ID NO. |
|---|---|---|---|---|---|---|---|---|
| way | Figs. | No. | number | Enzyme Candidate | Gene | (aa) | (nucleotide) | (protein) |
| A | 1, 2, | A1 | 2.3.1.54 | Formate-C acetyltransferase | PFLB | P75793 | 12 | 13 |
| | 3, 4 | | | | | | | |
| A | 1, 2, | A1 | 1.97.1.4 | Formate-C acetyltransferase | PFLA | C4ZXZ6 | 14 | 15 |
| | 3, 4 | | | (activating enzyme) | | | | |
| A | 1, 2, | A1 | 2.3.1.54 | Formate-C acetyltransferase | PFLB | K9LI23 | 16 | 17 |
| | 3, 4 | | | | | | | |
| A | 1, 2, | A1 | 1.97.1.4 | Formate-C acetyltransferase | PFLA | Q6RFH6 | 18 | 19 |
| | 3, 4 | | | (activating enzyme) | | | | |
| A | 1, 2, | A2 | 1.2.4.1 | Pyruvate dehydrogenase | pda1 | P16387 | 20 | 21 |
| | 3, 4 | | | complex | | | | |
| A | 1, 2, | A2 | 1.2.4.1 | Pyruvate dehydrogenase | pdb1 | P32473 | 22 | 23 |
| | 3, 4 | | | complex | | | | |
| A | 1, 2, | A2 | 2.3.1.12 | Pyruvate dehydrogenase | lat1 | P12695 | 24 | 25 |
| | 3, 4 | | | complex | | | | |
| A | 1, 2, | A2 | 1.8.1.4 | Pyruvate dehydrogenase | lpd1 | P09624 | 26 | 27 |
| | 3, 4 | | | complex | | | | |
| A | 1, 2, | A2 | N/A | Pyruvate dehydrogenase | pdx1 | P16451 | 28 | 29 |
| | 3, 4 | | | complex | | | | |
| A | 1, 2, | A2 | 1.2.4.1 | Pyruvate dehydrogenase | pdhA | F2MRX7 | 30 | 31 |
| | 3, 4 | | | complex (E1 aplha) | | | | |
| A | 1, 2, | A2 | 1.2.4.1 | Pyruvate dehydrogenase | pdhB | F2MRX8 | 32 | 33 |
| | 3, 4 | | | complex (E2 beta) | | | | |
| A | 1, 2, | A2 | 2.3.1.12 | Pyruvate dehydrogenase | aceF | F2MRX9 | 34 | 35 |
| | 3, 4 | | | complex (E2) | | | | |
| A | 1, 2, | A2 | 1.8.1.4 | Pyruvate dehydrogenase | lpd | F2MRY0 | 36 | 37 |
| | 3, 4 | | | complex (E3) | | | | |
| A | 1, 2, | B | 2.3.1.9 | acetyl coenzyme A | thlA | P45359 | 38 | 39 |
| | 3, 4 | | | acetyltransferase | | | | |
| A | 1, 2, | B | 2.3.1.9 | acetyl coenzyme A | Erg10 | P45359 | 40 | 41 |
| | 3, 4 | | | acetyltransferase | | | | |
| A | 1, 2, | C1 | 2.8.3.8 | Acetyl-CoA:acetoacetate-CoA | atoA | P76459 | 42 | 43 |
| | 3, 4 | | | transferase subunit | | | | |
| A | 1, 2, | C1 | 2.8.3.8 | Acetyl-CoA:acetoacetate-CoA | atoD | P76458 | 44 | 45 |
| | 3, 4 | | | transferase subunit | | | | |
| A | 1, 2, | C2 | 3.1.2.— | Acyl-CoAthioesterase 2 | atoA* | NA | 3 | N/A |
| | 3, 4 | | | | | | | |
| A | 1, 2, | D | 4.1.1.4 | acetoacetate decarboxylase | adc | P23670 | 46 | 47 |
| | 3, 4 | | | | | | | |
| A | 1, 2, | D | 4.1.1.4 | acetoacetate decarboxylase | adc | A6M020 | 48 | 49 |
| | 3, 4 | | | | | | | |
| A | 1, 2, | E | 1.1.1.2 | secondary alcohol | adh | P25984 | 50 | 51 |
| | 3, 4 | | | dehydrogenase | | | | |
| A | N/A | Temporary | 1.6.1.1. | Soluble pyridine nucleotide | udhA | P27306 | 104 | 105 |
| | | redox sink | | transhydrogenase | | | | |
| A | N/A | Temporary | 1.1.1.67 | Mannitol-2dehydrogenase | mdh | Q83VI5 | 106 | 107 |
| | | redox sink | | | | | | |
| B1/B2 | 1, 3 | F1 | 4.2.3.3 | methylglyoxal synthase | mgsA | P42980 | 52 | 53 |
| B1/B2 | 1, 3 | F1 | 4.2.3.3 | methylglyoxal synthase | mgsA | P0A731 | 54 | 55 |
| B1/B2 | 1, 3 | F2 | 4.2.3.3 | methylglyoxal synthase | mgsA* | P0A731 | 56 | 57 |
| B1/B2 | 1, 3 | G1, I1 | 1.1.1.6 | glycerol dehydrogenase | gldA | P0A9S5 | 58 | 59 |
| B1/B2 | 1, 3 | G2 | 1.1.1.283 | | gre2 | Q12068 | 60 | 61 |
| B1/B2 | 1, 3 | G3 | 1.1.1.21 | aldose reductase | gre3 | P38715 | 62 | 63 |
| B1/B2 | 1, 3 | G4 | 1.1.1.76/ | butanediol dehydrogenase | budC | Q9ZNN8 | 64 | 65 |
| | | | 1.1.1.304 | | | | | |
| B1/B2 | 1, 3 | G5 | 1.1.1.4 | butanediol dehydrogenase | bdh1 | P39714 | 66 | 67 |
| B1/B2 | 1, 3 | H1 | 1.1.1.— | Alcohol dehydrogenase | yqhD* | Q46856 | 68 | 69 |
| B1/B2 | 1, 3 | H2 | 1.1.1.— | methylglyoxal reductase | ydjg | P77256 | 70 | 71 |
| B1/B2 | 1, 3 | H3 | 1.1.1.— | methylglyoxal reductase | ypr1 | C7GMG9 | 72 | 73 |
| B1/B2 | 1, 3 | I2 | 1.1.1.304 | methylglyoxal reductase, | budC | Q48436 | 74 | 75 |
| | | | | multifunctional | | | | |
| B1/B2 | 1, 2, | J1 | 1.1.1.77 | lactaldehyde reductase | fucO | P0A9S1 | 76 | 77 |
| | 3, 4 | | | | | | | |
| B1/B2 | 1, 2, | J2 | 1.1.1.— | methylglyoxal reductase | yafB | P30863 | 78 | 79 |
| | 3, 4 | | | [multifunctional] | | | | |
| B2, C2 | 3, 4 | K1 | 4.2.1.30 | glycerol dehydratase | dhaB1 | Q8GEZ8 | 80 | 81 |
| B2, C2 | 3, 4 | K1 | 4.2.1.30 | glycerol dehydratase activator | dhaB2 | Q8GEZ7 | 82 | 83 |
| B2, C2 | 3, 4 | K2 | 4.2.1.30 | diol dehydratase | b1 | Q1A666 | 84 | 85 |
| B2, C2 | 3, 4 | K2 | 4.2.1.30 | diol dehydratase activator | b2 | Q1A665 | 86 | 87 |
| B2, C2 | 3, 4 | L | 1.1.1.1 | alcohol dehydrogenase | adh | C6PZV5 | 88 | 89 |
| C1/C2 | 2, 4 | M1 | 1.1.1.28 | D-Lactate dehydrogenase | ldhA | P52643 | 90 | 91 |
| C1/C2 | 2, 4 | M2 | 1.1.1.27 | L-Lactate dehydrogenase | ldhL2 | P59390 | 92 | 93 |
| C1/C2 | 2, 4 | M2 | 1.1.1.27 | L-lactate dehydrogenase | ldh2 | P19858 | 94 | 95 |
| C1/C2 | 2, 4 | N | 2.8.3.1 | propionate CoA-transferase* | pct | Q9L3F7 | 96 | 97 |
| C1/C2 | 2, 4 | O | 2.3.3.— | Lactoyl-CoA Synthase | ACS1 | Q01574 | 98 | 99 |
| C1/C2 | 2, 4 | P | 1.2.1.— | CoA-dependent | pduP | Q9XDN1 | 100 | 101 |
| | | | | propionaldehyde | | | | |
| | | | | dehydrogenase* | | | | |
| C1/C2 | 2, 4 | Q | 1.1.1.77 | L-1,2-propanediol | fucO | P0A9S1 | 102 | 103 |
| | | | | oxidoreductase | | | | |

Methods for the Co-Production of 2-Propanol and 1-Propanol and/or 1,2-Propanediol

2-propanol and 1-propanol and/or 1,2-propanediol may be produced by contacting any of the genetically modified microorganisms provided herein with a fermentable carbon source. Such methods may preferably comprise contacting a fermentable carbon source with a microorganism comprising one or more polynucleotides coding for enzymes in a pathway that catalyzes a conversion of the fermentable carbon source to any of the intermediates provided in FIGS. 1-4 (Tables 1-6) and one or more polynucleotides coding for enzymes in a pathway that catalyze a conversion of the one or more intermediates provided in FIGS. 1-4 (tables 1-6) to 2-propanol and 1-propanol and/or 1,2-propanediolin a fermentation media; and expressing the one or more polynucleotides coding for the enzymes in the pathway that catalyzes a conversion of the fermentable carbon source to the one or more intermediates provided in FIGS. 1-4 (tables 1-6) and one or more polynucleotides coding for enzymes in a pathway that catalyze a conversion of the one or more intermediates provided in FIGS. 1-4 (tables 1-6) to 2-propanol and 1-propanol and/or 1,2-propanediol.

The metabolic pathways that lead to the production of industrially important compounds involve oxidation-reduction (redox) reactions. For example, during fermentation, glucose is oxidized in a series of enzymatic reactions into smaller molecules with the concomitant release of energy. The electrons released are transferred from one reaction to another through universal electron carriers, such Nicotinamide Adenine Dinucleotide (NAD) and Nicotinamide Adenine Dinucleotide Phosphate (NAD(P)), which act as cofactors for oxidoreductase enzymes. In microbial catabolism, glucose is oxidized by enzymes using the oxidized form of the cofactors (NAD(P)+ and/or NAD+) thus generating reducing equivalents in the form of the reduced cofactor (NAD(P)H and NADH). In order for fermentation to continue, redox-balanced metabolism is required, i.e., the cofactors must be regenerated by the reduction of microbial cell metabolic compounds.

Microorganism-catalyzed fermentation for the production of natural products is a widely known application of biocatalysis. Industrial microorganisms can affect multistep conversions of renewable feedstocks to high value chemical products in a single reactor. Products of microorganism-catalyzed fermentation processes range from chemicals such as ethanol, lactic acid, amino acids and vitamins, to high value small molecule pharmaceuticals, protein pharmaceuticals, and industrial enzymes. In many of these processes, the biocatalysts are whole-cell microorganisms, including microorganisms that have been genetically modified to express heterologous genes.

Some key parameters for efficient microorganism-catalyzed fermentation processes include the ability to grow microorganisms to a greater cell density, increased yield of desired products, increased amount of volumetric productivity, removal of unwanted co-metabolites, improved utilization of inexpensive carbon and nitrogen sources, adaptation to varying fermenter conditions, increased production of a primary metabolite, increased production of a secondary metabolite, increased tolerance to acidic conditions, increased tolerance to basic conditions, increased tolerance to organic solvents, increased tolerance to high salt conditions and increased tolerance to high or low temperatures. Inefficiencies in any of these parameters can result in high manufacturing costs, inability to capture or maintain market share, and/or failure to bring fermented end-products to market.

The methods and compositions of the present disclosure can be adapted to conventional fermentation bioreactors (e.g., batch, fed-batch, cell recycle, and continuous fermentation).

In some embodiments, a microorganism (e.g., a genetically modified microorganism) as provided herein is cultivated in liquid fermentation media (i.e., a submerged culture) which leads to excretion of the fermented product(s) into the fermentation media. In one embodiment, the fermented end product(s) can be isolated from the fermentation media using any suitable method known in the art.

In some embodiments, formation of the fermented product occurs during an initial, fast growth period of the microorganism. In one embodiment, formation of the fermented product occurs during a second period in which the culture is maintained in a slow-growing or non-growing state. In one embodiment, formation of the fermented product occurs during more than one growth period of the microorganism. In such embodiments, the amount of fermented product formed per unit of time is generally a function of the metabolic activity of the microorganism, the physiological culture conditions (e.g., pH, temperature, medium composition), and the amount of microorganisms present in the fermentation process.

In some embodiments, the fermentation product is recovered from the periplasm or culture medium as a secreted metabolite. In one embodiment, the fermentation product is extracted from the microorganism, for example when the microorganism lacks a secretory signal corresponding to the fermentation product. In one embodiment, the microorganisms are ruptured and the culture medium or lysate is centrifuged to remove particulate cell debris. The membrane and soluble protein fractions may then be separated if necessary. The fermentation product of interest may then be purified from the remaining supernatant solution or suspension by, for example, distillation, fractionation, chromatography, precipitation, filtration, and the like.

The methods of the present disclosure are preferably preformed under anaerobic conditions. Both the degree of reduction of a product as well as the ATP requirement of its synthesis determines whether a production process is able to proceed aerobically or anaerobically. To produce 2-propanol and 1-propanol and/or 1,2-propanediol via anaerobic microbial conversion, or at least by using a process with reduced oxygen consumption, redox imbalances should be avoided. Several types of metabolic conversion steps involve redox reactions including some of the conversions as set forth in FIG. 1. Such redox reactions involve electron transfer mediated by the participation of redox cofactors such as NADH, NADPH and ferredoxin. Since the amounts of redox cofactors in the cell are limited to permit the continuation of metabolic processes, the cofactors have to be regenerated. In order to avoid such redox imbalances, alternative ways of cofactor regeneration may be engineered, and in some cases additional sources of ATP generation may be provided. Alternatively, oxidation and reduction processes may be separated spatially in bioelectrochemical systems (Rabaey and. Rozendal, 2010, Nature reviews, Microbiology, vol 8: 706-716).

In some embodiments, redox imbalances may be avoided by using substrates (e.g., fermentable carbon sources) that are more oxidized or more reduced. for example, if the utilization of a substrate results in a deficit or surplus of electrons, a requirement for oxygen can be circumvented by using substrates that are more reduced or oxidized, respectively. For example, glycerol which is a major byproduct of biodiesel production is more reduced than sugars, and is therefore more suitable for the synthesis of compounds whose production from sugar results in cofactor oxidation, such as succinic acid. In some embodiments, if the conversion of a substrate to a product results in an electron deficit, co-substrates can be added that function as electron donors (Babel 2009, Eng. Life Sci. 9,285-290). An important criterion for the anaerobic use of co-substrates is that their redox potential is higher than that of NADH (Geertman et al., 2006, FEMS Yeast Res. 6, 1193-1203). If the conversion of substrate to produce results in an electron surplus, co-substrates can be added that function as electron acceptors.

In some embodiments, a gene coding for an enzyme that can act as a temporary redox sink (i.e. by catalyzing a reduction reaction of a readily available substrate) is used in order to avoid redox imbalances. Some examples of genes that may act as redox sinks in yeast were described in Wang et al., 2012, Biochem. Eng. J. vol. 67, p 126-131. These enzymes include, but are not limited to, soluble pyridine transhydrogenases (EC number 1.6.1.1.) and water-forming NADH oxidase (EC number 1.6.3.4.).

### Methods for the Production of Polypropylene

1-propanol and 2-propanol produced via methods disclosed herein and without the need of separating one from the other may be dehydrated together to form propylene, which may then be polymerized to produce polypropylene in a cost-effective manner.

Propylene is a chemical compound that is widely used to synthesize a wide range of petrochemical products. For instance, this olefin is the raw material used for the production of polypropylene, its copolymers and other chemicals such as acrylonitrile, acrylic acid, epichloridrine and acetone. Propylene demand is growing faster than ethylene demand, mainly due to the growth of market demand for polypropylene. Propylene is polymerized to produce thermoplastics resins for innumerous applications such as rigid or flexible packaging materials, blow molding and injection molding.

Propylene is typically obtained in large quantity scales as a byproduct of catalytical or thermal oil cracking, or as a co-product of ethylene production from natural gas. (Propylene, Jamie G. Lacson, CEH Marketing Research Report-2004, Chemical Economics Handbook-SRI International). The use of alternative routes for the production of propylene has been continuously evaluated using a wide range of renewable raw materials (“Green Propylene”, Nexant, January 2009). These routes include, for example, dimerization of ethylene to yield butylene, followed by metathesis with additional ethylene to produce propylene. Another route is biobutanol production by sugar fermentation followed by dehydration and methatesis with ethylene. Some thermal routes are also being evaluated such as gasification of biomass to produce a syngas followed by synthesis of methanol, which may then produce green propylene via methanol-to-olefin technology.

Propylene production by 2-propanol dehydration has been well-described in document EP00498573B1, wherein all examples show propylene selectivity higher than 90% with high conversions. Dehydration of 1-propanol has also been studied in the following articles: “Mechanism and Kinetics of the Acid-Catalyzed Dehydration of 1- and iso-propanol in Hot Compressed Liquid Water” (Antal, M et al., Ind. Eng. Chem. Res. 1998, 37, 3820-3829) and “Fischer-Tropsch Aqueous Phase Refining by Catalytic Alcohol Dehydration” (Nel, R. et al., Ind. Eng. Chem. Res. 2007, 46, 3558-3565). The reported yield is higher than 90%.

Without further description, it is believed that one of ordinary skill in the art may, using the preceding description and the following illustrative examples, make and utilize the agents of the present disclosure and practice the claimed methods. The following working examples are provided to facilitate the practice of the present disclosure, and are not to be construed as limiting in any way the remainder of the disclosure.

### EXAMPLES

### Example 1

### Modification of a Microorganism to Render it Unable to Produce Ethanol, but Able to Grow on Glucose as the Sole Carbon Source Under Anaerobic Conditions

This example demonstrates the construction of yeast strain BRKY-272 (haploid and isogenic to Saccharomyces cerevisiae S288C) simultaneously expressing genes coding for enzymes in a pathway that catalyze the conversion of an acetyl-CoA intermediate to 2-propanol and genes coding for enzymes in a pathway that catalyzes the production of cytosolic acetyl-CoA intermediate from a pyruvate intermediate. The strain further comprises deletions of the PDC1, PDC5 and PDC6 genes coding for the three pyruvate decarboxylase isoforms, and thus lacks pyruvate decarboxylase activity and the capacity to produce ethanol. The strain further comprises an integration of a gene expressing the truncated version of the MTH1 enzyme as set forth in SEQ ID NO: 2.

The strains listed in Table 7 represent the step-wise creation of strain BRKY-272. All DNA-mediated transformation into S. cerevisiae was conducted using the Lithium Acetate procedure as described by Gietz R W and Woods R A, Guide to Yeast Gentics and Molecular Cell Biology. Part B. San Diego, Calif.: Academic Press Inc. pp. 87-96 (2002) and in all cases integration of the constructs was confirmed by PCR amplification ans sequencing of genomic DNA. In some cases, strains with more than one desired trait were obtained by crossing haploid strains of compatible mating types. In these cases, diploid construction, sporulation, tetrad dissection, and random spore analysis was performed according to Treco D A and Winston F, UNIT 13.2 Growth and Manipulation of Yeast, Curr. Protoc. Mol. Biol. 82:13.2.1-13.2.12 (2008).

Strains representing the step-wise creation of strain BRKY-272 that simultaneously expresses all genes coding for enzymes in a pathway that catalyze the conversion of an acetyl-CoA intermediate to 2-propanol and genes coding for enzymes in a pathway that catalyzes the production of cytosolic acetyl-CoA intermediate from a pyruvate intermediate are provided in Table 7 below. The strain further comprises deletions of the PDC1, PDC5 and PDC6 genes coding for the three pyruvate decarboxylase isoforms and the integration of a gene coding for the truncated version of the MTH1 gene.

TABLE 7
Strains for the step-wise creation of strain BRKY-272.

| Strain ID | Strain Genotype | Vectors | Host |
|---|---|---|---|
| FY23 | S288C, mating type a, ura3-52, leu2Δ1, trp1Δ63 | none | N/A |
| FY86 | S288C, mating type alpha, ura3-52, leu2Δ1, | none | N/A |
| | his3delta200 | | |
| BRKY-02 | S288C, mating type a, ura3-52, leu2Δ1, | none | FY23 |
| | trp1Δ63, PDC1::URA3 | | |
| BRKY-31 | S288C, mating type a; his3delta200, ura3-52, | none | FY23 × FY86 |
| | trp1delta63, leu2delta1 | | progeny |
| BRKY-37 | S288C, mating type alpha; his3delta200, ura3-52, | none | FY86 × BRKY02 |
| | trp1delta63, leu2delta1, PDC1::URA3 | | progeny |
| BRKY-69 | S288C, mating type a; his3delta200, ura3-52, | none | BRKY31 |
| | trp1delta63, leu2delta1, PDC6::URA3 | | |
| BRKY-86 | S288C, mating type a; his3delta200, ura3-52, | none | BRKY69 |
| | trp1delta63, leu2delta1, PDC6::URA3, locus Chr | | |
| | XI: 91575-adh/HIS3 | | |
| BRKY-97 | S288C, mating type a; his3delta200, ura3-52, | none | BRKY31 |
| | trp1delta63, leu2delta1, PDC5::KanMX4_tMTH1 | | |
| BRKY-115 | S288c, mating type α; his3delta200, ura3-52, | none | BRKY37 × BRKY97 |
| | trp1delta63, leu2delta1, PDC5::KanMX4_tMTH1 | | progeny |
| BRKY-118 | S288c, mating type alpha; his3delta200, ura3-52, | none | BRKY37 × BRKY97 |
| | trp1delta63, leu2delta1, | | progeny |
| | PDC5::KanMX4_tMTH1 PDC1::URA3 | | |
| BRKY-130 | S288C, mating type α; his3delta200, ura3-52, | none | BRKY115 × BRKY86 |
| | trp1delta63, leu2delta1, PDC5::KanMx4/MTH1, | | progeny |
| | PDC6::URA3, locus Chr XI: 91575-adh/HIS3 | | |
| BRKY-138 | S288C, mating type a; his3delta200, ura3-52, | none | BRKY86 × BRKY118 |
| | trp1delta63, leu2delta1, PDC1::URA3, | | progeny |
| | PDC5::KanMX4/MTH1, locus Chr XI: 91575- | | |
| | adh/HIS3 | | |
| BRKY-163 | S288C, mating type alpha; his3delta200, ura3-52, | none | BRKY138 × BRKY130 |
| | trp1delta63, leu2delta1, PDC1::URA3, | | progeny |
| | PDC5::KanMX4/MTH1, PDC6::URA3 locus Chr | | |
| | XI: 91575-adh/HIS3 | | |
| BRKY-174 | S288C, mating type alpha; his3delta200, ura3-52, | none | BRKY163 |
| | trp1delta63, leu2delta1, PDC1::URA3, | | |
| | PDC5::KanMX4/MTH1, PDC6::URA3, locus Chr | | |
| | XI: 91575-adh/HIS3, locus ChrX: 194944- | | |
| | atoA/atoD/TRP1 | | |
| BRKY-189 | S288C, mating type alpha; his3delta200, ura3-52, | none | BRKY174 |
| | trp1delta63, leu2delta1, PDC1::URA3, | | |
| | PDC5::KanMX4/MTH1, PDC6::URA3, locus Chr | | |
| | XI: 91575-adh/HIS3, ChrX: 194944- | | |
| | atoA/atoD/TRP1, locus | | |
| | YPRCtau3::thl/adc/natMX | | |
| BRKY 272 | S288C, mating type α; his3delta200, ura3-52, | pRS415-LEU2 with | BRKY189 |
| | trp1delta63, leu2delta1, PDC1::URA3, | genes pPGK1-PFLA | |
| | PDC5::KanMX4/MTH1, PDC6::URA3, locus Chr | (E. coli)-tADH1, | |
| | XI: 91575-adh/HIS3 isolate 1.1, ChrX: 194944- | pTEF-PFLB (E. coli)- | |
| | atoA/atoD/TRP1 isolate 10, locus | tTDH3, pTDH3- | |
| | YPRCtau3::thl/adc/natMX isolate 20.1; LEU2, | udhA-tADH1 | |
| | pPGK1-PFLA (E. coli)-tADH1, pTEF-PFLB | | |
| | (E. coli)-tTDH3, pTDH3-udhA-tADH1 | | |
| BRKY 397 | S288C, mating type α; his3delta200, ura3-52, | pRS415-LEU | BRKY189 |
| | trp1delta63, leu2delta1, pdc1::URA3, | (empty) | |
| | pdc5::KanMX4/MTH1, pdc6::URA3, locus Chr | | |
| | XI: 91575-adh/HIS3, ChrX: 194944- | | |
| | atoA/atoD/TRP1, locus | | |
| | YPRCtau3::thl/adc/natMX; LEU2 | | |

FY23 and FY86 are haploid strains isogenic to Saccharomyces cerevisiae S288C as described by Winston et al. 1995, Yeast, 11, issue 1, 53-55), each containing three auxotrophic markers, and were used as the “wild-type” strains for this study.

Strain BRKY-02 was obtained by deleting the PDC1 gene from strain FY23 with a URA3 marker in a linear construct. The linear construct was built by PCR amplification of the URA3 marker gene from the commercial vector pESC-URA with primers BK0592 and BK0593 (Table 8) containing 40 bp 5′ extensions corresponding to regions upstream and downstream of the PDC1 locus (SEQ ID NO: 4). Upon introduction in a S. cerevisiae host cell, this construct can integrated by homologous recombination into the PDC1 locus of the genome, functionally disrupting PDC1p by replacing the PDC1p coding sequence with its integrating sequence. The resulting strain was selected for uracyl prototrophy in Yeast Nitrogen Base Media without uracyl (Sigma) and confirmed by PCR amplification of genomic DNA.

Strains BRKY-31 and BRKY-37 were generated by crossing and tetrad dissection of strains FY23xFY86 and FY86xBRKY-02, respectively. The objective was to obtain haploid strains with four auxotrophic markers and, in the case of BRKY-37, the four auxotrophic markers and the PDC1::URA3 deletion.

Strain BRKY-69 was obtained by deleting the PDC6 gene from strain BRKY-31 with a URA3 marker in a linear construct. The linear construct was built by PCR amplification of the URA3 marker gene from the commercial vector pESC-URA with primers BK0678 and BK0679 (Table 8) containing 40 bp 5′ extensions corresponding to regions upstream and downstream of the PDC6 locus (SEQ ID NO:5). Upon introduction into a S. cerevisiae host cell, this construct can integrate by homologous recombination into the PDC6 locus of the genome, functionally disrupting PDC6p by replacing the PDC6p coding sequence with its integrating sequence. The resulting strain was selected for uracyl prototrophy in Yeast Nitrogen Base Media without uracyl (Sigma) and confirmed by PCR amplification of genomic DNA.

Strain BRKY-86 was obtained by integrating at locus Chr XI:91575-..92913 of strain BRKY-69 a construct for the expression of the secondary alcohol dehydrogenase from Clostridium beijerinckii (Table 6, Enzyme No. E, E.C. Number 1.1.1.2) controlled by the TEF1 promoter and the PGK1 terminator. The integration cassette was flanked by ˜150 bp homology regions for locus Chr XI: 91575..92913 and comprised the HIS3 auxotrophic marker for strain selection. The whole construct was built by overlapping PCR (SEQ ID NO:6). Upon introduction in a S. cerevisiae host cell, this construct can integrate by homologous recombination into the Chr XI:91575-..92913 locus of the genome. The resulting strain was selected for histidine prototrophy in Yeast Nitrogen Base Media without histidine (Sigma) and confirmed by PCR amplification and sequencing of genomic DNA.

Strain BKRY-97 was obtained by deleting the PDC5 gene from strain BRKY-31 with a linear construct comprising the KanMX4 marker gene and a expression cassette for the truncated version of the MTH1 enzyme set forth in SEQ ID NO:2 flanked by homology sequences consisting of the upstream and downstream nucleotide sequences of the S. cerevisiae PDC5 locus. A truncated version of the MTH1 gene was synthesized by IDT (Integrated DNA Technologies, Coralville, Iowa). Next, the truncated MTH1 gene was introduced into a surrogate plasmid vector carrying the KanMX4 selection marker flanked by loxp recognition sequences (pUG6 vector by Euroscarf). PCR amplification of the truncated MTH1 gene and KanMX4 marker gene from the surrogate vector was performed with primers BKO909 and BKO910 (Table 8) containing 5′ extensions corresponding to regions upstream and downstream of the PDC5 locus (SEQ ID NO:7). Upon introduction in a S. cerevisiae host cell, this construct can integrate by homologous recombination into the PDC5 locus of the genome, functionally disrupting PDC5p by replacing the PDC5p coding sequence with its integrating sequence for the concomitant expression of the truncated version of MTH1 and KanMX4. The resulting strain was selected for Geneticin resistance in YPD Media containing 150 micrograms per liter of Geneticin and confirmed by PCR amplification and sequencing of genomic DNA.

Strain BRKY-115 is a progeny of a cross between strains BRKY-37 and BRKY-97 and differs from its parent BRKY-97 only in the mating type. This different mating type was used in a further cross aimed at obtaining the PDC-null triple deletion strain.

Strain BRKY-118 is an ethanol-null strain carrying deletions in genes PDC1 and PDC5, coding for the two most important isoforms of pyruvate decarboxylases. Deletion of both genes causes significantly decreased amounts of ethanol from sugar and creates a C2-auxotrophy. After sequential deletion of the two genes failed repeatedly, we performed a cross between strains BRKY-37 and BRKY-97 and selected tetrads on the basis of uracyl prototrophy and Geneticin resistance and were able to obtain the PDC1/PDC5 double knockout.

Strain BRKY-130 is the progeny of a cross between BRKY115×BRKY86 and carries deletions of PDC6 and PDC5 with the concomitant expression of the truncated version of the MTH1 and the secondary alcohol dehydrogenase of C. beijerinckii of the 2-propanol pathway.

Strain BRKY-138 is the progeny of a cross between BRKY86 and BRKY118 and carries deletions of PDC1 and PDC5 with the concomitant expression of the truncated version of the MTH1 and the secondary alcohol dehydrogenase of C. beijerinckii of the 2-propanol pathway.

Strain BRKY-163 is the PDC-null strain with the concomitant expression of the truncated version of the MTH1 gene and the secondary alcohol dehydrogenase of C. beijerinckii. This strain was obtained as the progeny of a cross between BRKY-130 and BRKY-138 with the tetrads being screened by PCR for deletions in both PDC1 and PDC6 loci, since both the PDC5 deletion (containing the truncated MTH1 gene) and the secondary alcohol dehydrogenase integration were inherited from both parents and as such were not subject to segregation.

Strain BRKY-174 is a PDC-null strain with the concomitant expression of the truncated version of the MTH1 gene, the secondary alcohol dehydrogenase of C. beijerinckii, and the two subunits of the acetyl-CoA acetoacetate CoA transferase of E. coli. Strain BRKY-174 was obtained by integrating at locus Chr X: 194944..195980 of strain BRKY-163 a construct for the expression of the gene atoA from E. coli controlled by the TEF1 promoter and CYC1 terminator and the atoD gene from E. coli controlled by the PGK1 promoter and ADH1 terminator, which code for the two subunits of the acetyl-CoA acetoacetate CoA transferase from E. coli (Enzyme No. C1, E.C. Number 2.8.3.8, Table 6). The integration cassette was flanked by ˜150 bp homology regions for locus Chr X: 194944..195980 and comprised the TRP1 auxotrophic marker for strain selection and was built by overlapping PCR (SEQ ID NO:8). Upon introduction in a S. cerevisiae host cell, this construct can integrate by homologous recombination into the locus Chr X: 194944..195980 locus of the genome. The resulting strain was selected for triptophan prototrophy in Yeast Nitrogen Base Media without tryptophan (Sigma) and confirmed by PCR amplification and sequencing of genomic DNA.

Strain BRKY-189 is a PDC-null strain with the concomitant expression of the truncated version of the MTH1 gene, the secondary alcohol dehydrogenase of C. beijerinckii, the two subunits of the acetyl-CoA acetoacetate CoA transferase from E. coli, the thiolase gene from Clostridium acetobutylicum, and the acetoacetate decarboxylase gene from C. beijerinckii, thus expressing a full 2-propanol producing pathway from acetyl-CoA. Strain BRKY-189 was obtained by integrating at locus YPRCtau3 of strain BRKY-174 a construct for the expression of the thiolase gene from C. acetobutylicum (Enzyme No. B, E.C. number 2.3.1.9, Table 6) controlled by the ADH1 promoter and TEF1 terminator and the acetoacetate decarboxylase gene from C. beijerinckii (Enzyme No. D, E.C. number 4.1.1.4, Table 6) controlled by the TDH3 promoter and TRP1 terminator. The integration cassette was flanked by ˜150 bp homology regions for locus YPRCtau3 and comprised the Nourseotricin marker for strain selection and was bilt by overlapping PCR (SEQ ID NO:9). Upon introduction in a S. cerevisiae host cell, this construct can integrate by homologous recombination into the YPRCtau3 locus of the genome. The resulting strain was selected for nourseotricin resistance in YPD Media supplemented with 100 micrograms per mililiter of nourseotricin and confirmed by PCR amplification and sequencing of genomic DNA.

Strain BRKY-272 was obtained by transforming strain BRKY-189 with a single copy plasmid (pRS415-LEU backbone, ATCC® 87520™) expressing the following genes and control sequences: 1) PFLA gene from E. coli controlled by the PGK1 promoter and ADH1 terminator, 2) PFLB gene from E. coli controlled by the TEF1 promoter and TDH3 terminator, and 3) udhA gene from E. coli controlled by the TDH3 promoter and ADH1 terminator. The resulting plasmid sequence is included as SEQ ID NO:10. The resulting strain thus comprises a full pathway for the conversion of the cytosolic pyruvate intermediate into cytosolic acetyl-CoA in addition to the full pathway for the conversion of the acetyl-CoA intermediate into 2-propanol integrated in the genome of the BRKY-189 parent strain. Strain BRKY-272 is thus a 2-propanol and ethanol-null production strain able to produce cytosolic acetyl-CoA.

Strain BRKY-397 was obtained transforming strain BRKY-189 with a single copy plasmid (pRS415-LEU backbone, ATCC® 87520™) that cofers prototrophy to leucine and was used as a control strain.

TABLE 8
Oligonucleotides used to build strains by
homologous recombination

| Name | DNA Sequence (5′→3′) |
|---|---|
| BKO592 | ACTCATAACCTCACGCAAAATAACACAGTCAAATCAATCAAA |
| | AGCTTTTCAATTCAATTCATC (SEQ ID NO: 108) |
| BKO593 | AATGCTTATAAAACTTTAACTAATAATTAGAGATTAAATCGC |
| | GGGTAATAACTGATATAAT (SEQ ID NO: 109) |
| BKO678 | CCTAGATCGATTTGATTACAGGATAAGGGATATGGTGCGATT |
| | CGCGAGCTTTTACCAATATGTATAAAAGGCGGCTGTTTGAAG |
| | CCATTCTATCTTAATCTTGTGCTATTGCAGTCCTCTTTTATA |
| | TACAGTATAAATAAAAAACCCACGTAATATAGCAAAAACATA |
| | TTGCCAACAAAAGCTTTTCAATTCAATTCATC |
| | (SEQ ID NO: 110) |
| BKO679 | GGGTAATAACTGATATAATGCCATTAGTAGTGTACTCAAACG |
| | AATTATTGTTGCAAATAAATAAACTTACACAGTTTGAATACA |
| | TAAATCAATCAGACAAATAAATACATCGGTTCAAATTATACT |
| | AAATCTAAATACTACGTTATCGCCGTGAATTACGCAATTCGC |
| | ATGTTACGTACTGCGCGTCTCTTGTTGAATA |
| | (SEQ ID NO: 111) |
| BKO909 | TCAAGTTCCTCGATACTAGTTATTTGTAATACGTATACGAAT |
| | TCCTTCAACAAAGGCCAAGGAAATAAAGCAAATAACAATAAC |
| | ACCATTATTTTAATTTTTTTTCTATTACTGTCGCTAACACCT |
| | GTATGGTTGCAACCAGGTGAGAATCCTTCTGATGCATACTTT |
| | ATGCGTTTATGCTACGCTGCAGGTCGACAA |
| | (SEQ ID NO: 112) |
| BKO910 | GCTAAAGGTACAAAACCGAATACGAAAGTAAATAAATTAATC |
| | AGCATAAAATTAAATAATAAACCACCTAAAATATTAGAAGCT |
| | AATCTTTAACCTGGAAGACAGGACAGAAAAGTAATTACAAGA |
| | ACATATGTGAAAAAAAATAGTTGATATTTTAAACCAAATCAG |
| | AAATTTATTATACAGAGCGCCCAATACGCAAA |
| | (SEQ ID NO: 113) |

By providing a PDC-null strain with 1) a truncated MTH1 gene to alleviate 2C auxotrophy and increase glucose tolerance (Oud et al. Microbial Cell Factories, vol. 11, 2012, p. 131-140); 2) a pathway for the production of cytosolic acetyl-CoA from pyruvate (Pyruvate Formate Lyase and PFL-activating enzyme coding genes from E. coli) that is only functional under anaerobic conditions; and 3) a temporary redox sink (udhA transhydrogenase coding gene from E. coli) that would enable reoxidation of the excess NADH produced at the end of glycolysis, the strain should be able to grow under anaerobic conditions. This would be different from parental strain BRKY-189 that lacks a cytosolic acetyl-CoA production pathway and is redox imbalanced under anaerobic conditions, and, thus, it is not expected to grow under strict anaerobic conditions.

To test this hypothesis, strains BRKY-397 and BRKY-272 were cultured in YNB Media without leucine (to select for the pRS415 plasmid) containing 8 g/L of glucose as the sole carbon source. The full composition of the media follows: Glucose, 8 g/L, Ammonium sulfate, 5.0 g/L, Biotin, 2.0 micrograms/L, Calcium pantothenate, 400 micrograms/L, Folic acid, 2.0 micrograms/L, Inositol, 2.0 mg/L, Nicotinic acid, 400 micrograms/L, p-Aminobenzoic acid, 200 micrograms/L, Pyridoxine HCl, 400 micrograms/L, Riboflavin, 200 micrograms/L, Thiamine HCL, 400 micrograms/L, Citric acid, 0.1 g/L, Boric acid, 500 micrograms/L, Copper sulfate, 40 micrograms/L, Potassium iodide, 100 micrograms/L, Ferric chloride, 200 micrograms/L, Magnesium sulfate, 400 micrograms/L, Sodium molybdate, 200 micrograms/L, Zinc sulfate, 400 micrograms/L, Potassium phosphate monobasic, 1.0 g/L, Magnesium sulfate, 0.5 g/L, Sodium chloride, 0.1 g/L, Calcium chloride, 0.1 g/L, all standard amino acids except for leucine at a concentration of 76 mg/L, Adenine, 18 mg/L, inositol, 76 mg/L, p-aminobenzoic acid (8 mg/L), uracil (76 mg/L). All reagents for media preparation were obtained from Sigma (YNB without amino acids, Part No. Y0626, Yeast Synthetic Drop-out Medium Supplements without leucine, Part No. Y1376).

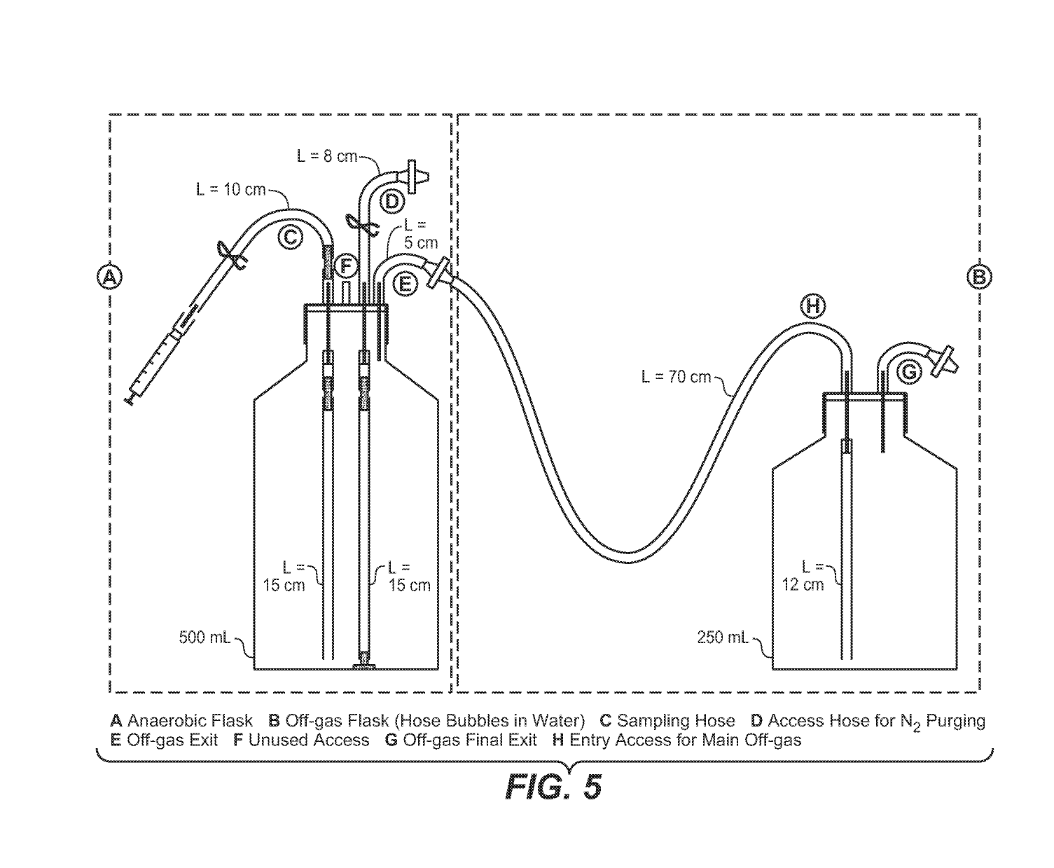

The strains were first grown in aerobic shake flasks for 24 hours until reaching an OD₆₀₀=˜4. Cells from this aerobic pre-culture were inoculated to an OD600 of ˜0.3 in sealed shake flasks purged with N₂ gas for 20 minutes before and immediately after inoculation. To prevent positive pressure build-up from CO₂ production, the flasks were provided with an off-gas tube that bubbled into a glass bottle containing sterilized water. The strains were cultured for up to 300 h. Samples were taken daily through a built-in sampling syringe. FIG. 5 shows a schematic representation of the anaerobic flask system.

Cell biomass was calculated by measuring the absorbance at 600 nm in a ULTROSPEC 2000 spectrophotometer UV/visible (Pharmacia Biotech) after appropriate dilution in saline. For HPLC-RI analysis, the samples were filtered through a 0.2 μm filter (Millipore). pyruvic, lactic and acetic acids, ethanol, glycerol, 2-propanol, 1,2-propanediol and sugars were separated and quantified by high-performance liquid chromatography (Waters 600 Chromatograph), using an ion exclusion column Aminex HPX-87H (Bio-Rad). Operating conditions were: 0.04 mol L⁻¹ H₂SO₄ degassed eluent, flow rate 0.6 mL min⁻¹, column temperature 35° C. and refractometer temperature 35° C.

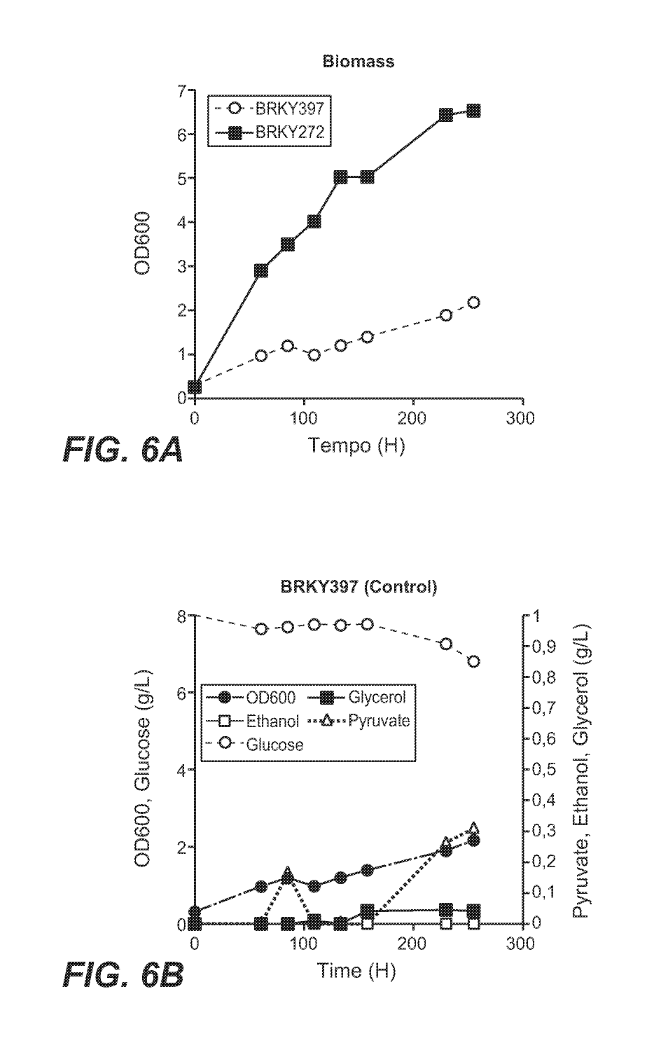

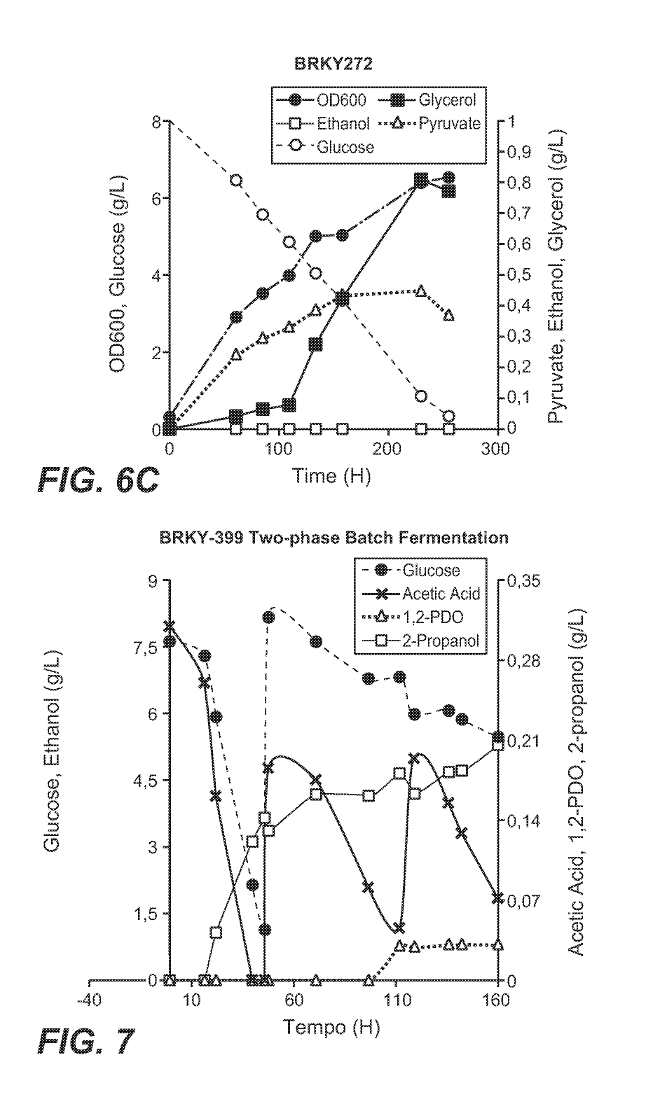

FIG. 6A shows increased growth of the BRKY-272 strain compared to the control (BRKY-397) in the anaerobic flask system shown in FIG. 5. The marginal growth of the control strain (BRKY-397) is hypothetized to have resulted from traces of oxygen present in the anaerobic flask system. However, we show that the growth of strain BRKY-272 is significantly increased compared to the control, as well as the consumption of glucose and production of metabolites as shown in FIGS. 6B (control strain BRKY-397) and FIG. 6C (test strain BRKY-272). None of the strains produced ethanol, thus confirming the PDC-null genotype. The results shown in FIG. 6 are representative of at least three independent replicates. Although strain BRKY-272 contains all genes in a pathway for the convertion of the acetyl-CoA intermediate into 2-propanol, the production of 2-propanol was not expected due to the absence of acetate that would act as an acceptor for the acetoacetyl-CoA:acetate CoA transferase (atoAD from E. coli) or a Acetoacetyl-CoA acyl-CoA thiolase.

Example 1 thus shows that it was possible to restore anaerobic growth a PDC-null yeast strain by providing three key elements: 1) a truncated MTH1 gene to alleviate 2C auxotrophy and increase glucose tolerance (Oud et al. Microbial Cell Factories, vol. 11, 2012, p. 131-140); 2) a pathway for the production of cytosolic acetyl-CoA from pyruvate (Pyruvate Formate Lyase and PFL-activating enzyme coding genes from E. coli) that is only functional under anaerobic conditions; and 3) a temporary redox sink (udhA transhydrogenase coding gene from E. coli) that would enable reoxidation of the excess NADH produced at the end of glycolysis.

### Example 2

### Modification of a Microorganism for Production of 1,2-Propanediol and 2-Propanol Without Production of Ethanol

This example demonstrates the construction of yeast strain BRKY-399 (haploid and isogenic to Saccharomyces cerevisiae S288C) simultaneously expressing genes coding for enzymes in a pathway that catalyze the conversion of an acetyl-CoA intermediate to 2-propanol and a dihydroxyacetone intermediate to 1,2-propanediol. The strain further comprises deletions of the PDC1, PDC5 and PDC6 genes coding for the three pyruvate decarboxylase isoforms, and thus lacks pyruvate decarboxylase activity. The strain further comprises an integration of a gene expressing the truncated version of the MTH1 enzyme as set forth in SEQ ID NO: 2.

The strains listed in Table 9 represent the step-wise creation of strain BRKY-399. All DNA-mediated transformation into S. cerevisiae was conducted using the Lithium Acetate procedure as described by Gietz R W and Woods R A, Guide to Yeast Gentics and Molecular Cell Biology. Part B. San Diego, Calif.: Academic Press Inc. pp. 87-96 (2002) and in all cases integration of the constructs was confirmed by PCR amplification of genomic DNA. In some cases, strains with more than one desired trait were obtained by crossing haploid strains of compatible mating types. In these cases, diploid construction, sporulation, tetrad dissection, and random spore analysis was performed according to Treco D A and Winston F, UNIT 13.2 Growth and Manipulation of Yeast, Curr. Protoc. Mol. Biol. 82:13.2.1-13.2.12 (2008).

TABLE 9
Strains representing the step-wise creation of strain BRKY-399 that
simultaneously expresses all genes coding for enzymes in a pathway that
catalyze the conversion of an acetyl-CoA intermediate to 2-propanol and a
dihydroxyacetone intermediate to 1,2-propanediol and further comprises
deletions of the PDC1, PDC5 and PDC6 genes coding for the three pyruvate
decarboxylase isoforms and the integration of a gene coding for the
truncated version of the MTH1 gene

| Strain ID | Strain Genotype | Vectors | Host |
|---|---|---|---|
| FY23 | S288C, mating type a, ura3-52, leu2Δ1, trp1Δ63 | none | N/A |
| FY86 | S288C, mating type alpha, ura3-52, leu2Δ1, | none | N/A |
| | his3delta200 | | |
| BRKY-02 | S288C, mating type a, ura3-52, leu2Δ1, trp1Δ63, | none | FY23 |
| | PDC1::URA3 | | |
| BRKY-31 | S288C, mating type a; his3delta200, ura3-52, | none | FY23 × FY86 |
| | trp1delta63, leu2delta1 | | progeny |
| BRKY-37 | S288C, mating type alpha; his3delta200, ura3-52, | none | FY86 × BRKY02 |
| | trp1delta63, leu2delta1, PDC1::URA3 | | progeny |
| BRKY-69 | S288C, mating type a; his3delta200, ura3-52, | none | BRKY31 |
| | trp1delta63, leu2delta1, PDC6::URA3 | | |
| BRKY-86 | S288C, mating type a; his3delta200, ura3-52, | none | BRKY69 |
| | trp1delta63, leu2delta1, PDC6::URA3, locus Chr | | |
| | XI: 91575-adh/HIS3 | | |
| BRKY-97 | S288C, mating type a; his3delta200, ura3-52, | none | BRKY31 |
| | trp1delta63, leu2delta1, PDC5:KanMX4_tMTH1 | | |
| BRKY-115 | S288c, mating type α; his3delta200, ura3-52, | none | BRKY37 × BRKY97 |
| | trp1delta63, leu2delta1, PDC5:KanMX4_tMTH1 | | progeny |
| BRKY-118 | S288c, mating type alpha; his3delta200, ura3-52, | none | BRKY37 × BRKY97 |
| | trp1delta63, leu2delta1, PDC5:KanMX4_tMTH1 | | progeny |
| | PDC1::URA3 | | |
| BRKY-130 | S288C, mating type α; his3delta200, ura3-52, | none | BRKY115 × BRKY86 |
| | trp1delta63, leu2delta1, PDC5::KanMx4/MTH1, | | progeny |
| | PDC6::URA3, locus Chr XI: 91575-adh/HIS3 | | |
| BRKY-138 | S288C, mating type a; his3delta200, ura3-52, | none | BRKY86 × BRKY118 |
| | trp1delta63, leu2delta1, PDC1::URA3, | | progeny |
| | PDC5::KanMX4/MTH1, locus Chr XI: 91575- | | |
| | adh/HIS3 | | |
| BRKY-163 | S288C, mating type alpha; his3delta200, ura3-52, | none | BRKY138 × BRKY130 |
| | trp1delta63, leu2delta1, PDC1::URA3, | | progeny |
| | PDC5::KanMX4/MTH1, PDC6::URA3 locus Chr | | |
| | XI: 91575-adh/HIS3 | | |
| BRKY-174 | S288C, mating type alpha; his3delta200, ura3-52, | none | BRKY163 |
| | trp1delta63, leu2delta1, PDC1::URA3, | | |
| | PDC5::KanMX4/MTH1, PDC6::URA3, locus Chr | | |
| | XI: 91575-adh/HIS3, locus ChrX: 194944- | | |
| | atoA/atoD/TRP1 | | |
| BRKY-189 | S288C, mating type alpha; his3delta200, ura3-52, | none | BRKY174 |
| | trp1delta63, leu2delta1, PDC1::URA3, | | |
| | PDC5::KanMX4/MTH1, PDC6::URA3, locus Chr | | |
| | XI: 91575-adh/HIS3, ChrX: 194944- | | |
| | atoA/atoD/TRP1, locus YPRCtau3::thl/adc/natMX | | |
| BRKY 399 | S288C, mating type alpha; his3delta200, ura3-52, | pRS415−LEU− | BRKY189 |
| | trp1delta63, leu2delta1, PDC1::URA3, | PGK1+yqhd*+tADH1, | |
| | PDC5::KanMX4/MTH1, PDC6::URA3, locus Chr | pPGK1+Gre2*+tCYC1, | |
| | XI: 91575-adh/HIS3, ChrX: 194944- | pTPI1+mgsA−Bs+tTDH3, | |
| | atoA/atoD/TRP1, locus YPRCtau3::thl/adc/natMX; | pTPI1+mgsA−Bs+tTDH3, | |
| | LEU2, pPGK1+yqhd*+tADH1, | pTPI1+mgsA−Bs+tTDH3, | |
| | pPGK1+Gre2*+tCYC1, pTPI1+mgsA−Bs+tTDH3, | pTEF1+udhA_Ec_tTDH3 | |
| | pTPI1+mgsA−Bs+tTDH3, pTPI1+mgsA−Bs+tTDH3, | | |
| | pTEF1+udhA_ Ec_tTDH3 | | |

FY23 and FY86 are haploid strains isogenic to Saccharomyces cerevisiae S288C as described by Winston et al. 1995, Yeast, 11, issue 1, 53-55), each containing three auxotrophic markers, and were used as the “wild-type” strains for this study.

Strain BRKY-02 was obtained by deleting the PDC1 gene from strain FY23 with a URA3 marker in a linear construct. The linear construct was built by PCR amplification of the URA3 marker gene from the commercial vector pESC-URA with primers BK0592 and BKO593 (Table 8) containing 40 bp 5′ extensions corresponding to regions upstream and downstream of the PDC1 locus (SEQ ID NO: 4). Upon introduction in a S. cerevisiae host cell, this construct can integrated by homologous recombination into the PDC1 locus of the genome, functionally disrupting PDC1p by replacing the PDC1p coding sequence with its integrating sequence. The resulting strain was selected for uracyl prototrophy in Yeast Nitrogen Base Media without uracyl (Sigma) and confirmed by PCR amplification of genomic DNA.

Strains BRKY-31 and BRKY-37 were generated by crossing and tetrad dissection of strains FY23xFY86 and FY86xBRKY-02, respectively. The objective was to obtain haploid strains with four auxotrophic markers and, in the case of BRKY-37, the four auxotrophic markers and the PDC1::URA3 deletion.

Strain BRKY-69 was obtained by deleting the PDC6 gene from strain BRKY-31 with a URA3 marker in a linear construct. The linear construct was built by PCR amplification of the URA3 marker gene from the commercial vector pESC-URA with primers BK0678 and BK0679 (Table 8) containing 40 bp 5′ extensions corresponding to regions upstream and downstream of the PDC6 locus (SEQ ID NO:5). Upon introduction in a S. cerevisiae host cell, this construct can integrate by homologous recombination into the PDC6 locus of the genome, functionally disrupting PDC6p by replacing the PDC6p coding sequence with its integrating sequence. The resulting strain was selected for uracyl prototrophy in Yeast Nitrogen Base Media without uracyl (Sigma) and confirmed by PCR amplification of genomic DNA.

Strain BRKY-86 was obtained by integrating at locus Chr XI:91575-..92913 of strain BRKY-69 a construct for the expression of the secondary alcohol dehydrogenase from Clostridium beijerinckii (Table 6, Enzyme No. E, E.C. Number 1.1.1.2) controlled by the TEF1 promoter and the PGK1 terminator. The integration cassette was flanked by ˜150 bp homology regions for locus Chr XI: 91575..92913 and comprised the HIS3 auxotrophic marker for strain selection. The whole construct was built by overlapping PCR (SEQ ID NO:6). Upon introduction in a S. cerevisiae host cell, this construct can integrate by homologous recombination into the Chr XI:91575-..92913 locus of the genome. The resulting strain was selected for histidine prototrophy in Yeast Nitrogen Base Media without histidine (Sigma) and confirmed by PCR amplification and sequencing of genomic DNA.

Strain BKRY-97 was obtained by deleting the PDC5 gene from strain BRKY-31 with a linear construct comprising the KanMX4 marker gene and a expression cassette for the truncated version of the MTH1 enzyme set forth in SEQ ID NO: 2 flanked by homology sequences consisting of the upstream and downstream nucleotide sequences of the S. cerevisiae PDC5 locus. A truncated version of the MTH1 gene was synthesized by IDT (Integrated DNA Technologies, Coralville, Iowa). Next, the truncated MTH1 gene was introduced into a surrogate plasmid vector carrying the KanMX4 selection marker flanked by loxp recognition sequences (pUG6 vector by Euroscarf). PCR amplification of the truncated MTH1 gene and KanMX4 marker gene from the surrogate vector was performed with primers BKO909 and BKO910 (Table 8) containing 5′ extensions corresponding to regions upstream and downstream of the PDC5 locus (SEQ ID NO:7). Upon introduction in a S. cerevisiae host cell, this construct can integrate by homologous recombination into the PDC5 locus of the genome, functionally disrupting PDC5p by replacing the PDC5p coding sequence with its integrating sequence for the concomitant expression of the truncated version of MTH1 and KanMX4. The resulting strain was selected for Geneticin resistance in YPD Media containing 150 micrograms per liter of Geneticin and confirmed by PCR amplification and sequencing of genomic DNA.

Strain BRKY-115 is a progeny of a cross between strains BRKY-37 and BRKY-97 and differs from its parent BRKY-97 only in the mating type. This different mating type was used in a further cross aimed at obtaining the PDC-null triple deletion strain.

Strain BRKY-118 is an ethanol-null strain carrying deletions in genes PDC1 and PDC5, coding for the two most important isoforms of pyruvate decarboxylases. Deletion of both genes causes significantly decreased amounts of ethanol from sugar and creates a C2-auxotrophy. After sequential deletion of the two genes failed repeatedly, we performed a cross between strains BRKY-37 and BRKY-97 and selected tetrads on the basis of uracyl prototrophy and Geneticin resistance and were able to obtain the PDC1/PDC5 double knockout.

Strain BRKY-130 is the progeny of a cross between BRKY115×BRKY86 and carries deletions of PDC6 and PDC5 with the concomintant expression of the truncated version of the MTH1 and the secondary alcohol dehydrogenase of C. beijerinckii of the 2-propanol pathway.

Strain BRKY-138 is the progeny of a cross between BRKY86 and BRKY118 and carries deletions of PDC1 and PDC5 with the concomitant expression of the truncated version of the MTH1 and the secondary alcohol dehydrogenase of C. beijerinckii of the 2-propanol pathway.

Strain BRKY-163 is the PDC-null strain with the concomitant expression of the truncated version of the MTH1 gene and the secondary alcohol dehydrogenase of C. beijerinckii. This strain was obtained as the progeny of a cross between BRKY-130 and BRKY-138 with the tetrads being screened by PCR for deletions in both PDC1 and PDC6 loci, since both the PDC5 deletion (containing the truncated MTH1 gene) and the secondary alcohol dehydrogenase integration were inherited from both parents and as such were not subject to segregation.

Strain BRKY-174 is a PDC-null strain with the concomitant expression of the truncated version of the MTH1 gene, the secondary alcohol dehydrogenase of C. beijerinckii, and the two subunits of the acetyl-CoA acetoacetate CoA transferase of E. coli. Strain BRKY-174 was obtained by integrating at locus Chr X: 194944..195980 of strain BRKY-163 a construct for the expression of the gene atoA from E. coli controlled by the TEF1 promoter and CYC1 terminator and the atoD gene from E. coli controlled by the PGK1 promoter and ADH1 terminator, which code for the two subunits of the acetyl-CoA acetoacetate CoA transferase from E. coli (Enzyme No. C1, E.C. Number 2.8.3.8, Table 6). The integration cassette was flanked by ˜150 bp homology regions for locus Chr X: 194944..195980 and comprised the TRP1 auxotrophic marker for strain selection and was built by overlapping PCR (SEQ ID NO:8). Upon introduction in a S. cerevisiae host cell, this construct can integrate by homologous recombination into the locus Chr X: 194944..195980 locus of the genome. The resulting strain was selected for triptophan prototrophy in Yeast Nitrogen Base Media without tryptophan (Sigma) and confirmed by PCR amplification and sequencing of genomic DNA.

Strain BRKY-189 is a PDC-null strain with the concomitant expression of the truncated version of the MTH1 gene, the secondary alcohol dehydrogenase of C. beijerinckii, the two subunits of the acetyl-CoA acetoacetate CoA transferase from E. coli, the thiolase gene from Clostridium acetobutylicum, and the acetoacetate decarboxylase gene from C. beijerinckii, thus expressing a full 2-propanol producing pathway from acetyl-CoA. Strain BRKY-189 was obtained by integrating at locus YPRCtau3 of strain BRKY-174 a construct for the expression of the thiolase gene from C. acetobutylicum (Enzyme No. B, E.C. number 2.3.1.9, Table 6) controlled by the ADH1 promoter and TEF1 terminator and the acetoacetate decarboxylase gene from C. beijerinckii (Enzyme No. D, E.C. number 4.1.1.4, Table 6) controlled by the TDH3 promoter and TRP1 terminator. The integration cassette was flanked by ˜150 bp homology regions for locus YPRCtau3 and comprised the Nourseotricin marker for strain selection and was bilt by overlapping PCR (SEQ ID NO:9). Upon introduction in a S. cerevisiae host cell, this construct can integrate by homologous recombination into the YPRCtau3 locus of the genome. The resulting strain was selected for nourseotricin resistance in YPD Media supplemented with 100 micrograms per mililiter of nourseotricin and confirmed by PCR amplification and sequencing of genomic DNA.

Strain BRKY-397 was obtained transforming strain BRKY-189 with a single copy plasmid (pRS415-LEU backbone, ATCC® 87520™) that cofers prototrophy to leucine and was used as a control strain.

Strain BRKY-399 was obtained by transforming strain BRKY-189 with a single copy plasmid (pRS415-LEU backbone, ATCC® 87520™) expressing the following genes: 1) three copies of the Bacillus subtilis mgsA (Enzyme No. F1, E.C. number 4.2.3.3), each controlled by the TPI1 promoter and the TDH3 terminator, 2) one copy of the yqhD gene from E. coli controlled by the PGK1 promoter and ADH1 terminator, 3) one copy of the GRE2 gene from S. cerevisiae controlled by the PGK1 promoter and CYC1 terminator, and 4) one copy of the udhA gene from E. coli controlled by the TDH3 promoter and ADH1 terminator. The resulting plasmid sequence is provided in SEQ ID NO:11. The resulting strain BRKY-399 thus comprises a full pathway for the conversion of the dihydroxyacetone-phosphate intermediate into 1,2-propanediol in addition to the full pathway for the conversion of the acetyl-CoA intermediate into 2-propanol integrated in the genome of the BRKY-189 parent strain. Strain BRKY-399 is thus an ethanol-null 2-propanol and 1,2-propanediol co-production strain.

### Example 3

### Fermentation of Glucose by Genetically Modified Ethanol-Null Microorganism to Produce 1,2-Propanediol and 2-Propanol

In this example, a genetically modified yeast strain BRKY-399, as produced in Example 2 above, was used to ferment a C6 sugar as a sole carbon source (glucose) and a C2 carbon source (sodium acetate) to co-produce 1,2-propanediol and 2-propanol in a two phase culture system in bioreactors. Since strain BRKY-399 lacks a functional pathway for the production of the acetyl-CoA intermediate from the pyruvate intermediate (i.e. PFL enzyme from E. coli), the culture media must be supplied with potassium acetate to serve as a substrate for acetyl-CoA formation from either the acetyl-CoA synthase or the acetoacetyl-CoA: acetate CoA transferase.

Strain BRKY-399 was cultured in YNB Media without leucine (to select for the pRS415 plasmid) containing 8 g/L of glucose and 0.5 g/L sodium acetate as the carbon sources. The full composition of the media follows: Glucose, 8 g/L, Sodium acetate, 0.5 g/L, Ammonium sulfate, 5.0 g/L, Biotin, 2.0 micrograms/L, Calcium pantothenate, 400 micrograms/L, Folic acid, 2.0 micrograms/L, Inositol, 2.0 mg/L, Nicotinic acid, 400 micrograms/L, p-Aminobenzoic acid, 200 micrograms/L, Pyridoxine HCl, 400 micrograms/L, Riboflavin, 200 micrograms/L, Thiamine HCL, 400 micrograms/L, Citric acid, 0.1 g/L, Boric acid, 500 micrograms/L, Copper sulfate, 40 micrograms/L, Potassium iodide, 100 micrograms/L, Ferric chloride, 200 micrograms/L, Magnesium sulfate, 400 micrograms/L, Sodium molybdate, 200 micrograms/L, Zinc sulfate, 400 micrograms/L, Potassium phosphate monobasic, 1.0 g/L, Magnesium sulfate, 0.5 g/L, Sodium chloride, 0.1 g/L, Calcium chloride, 0.1 g/L, all standard amino acids except for leucine at a concentration of 76 mg/L, Adenine, 18 mg/L, inositol, 76 mg/L, p-aminobenzoic acid (8 mg/L), uracil (76 mg/L). All reagents for media preparation were obtained from Sigma (YNB without amino acids, Part No. Y0626, Yeast Synthetic Drop-out Medium Supplements without leucine, Part No. Y1376).

Free-cell batch fermentation was conducted in a 0.6 L bioreactor (Multifors—Infors) containing 0.4 L of the sterile medium inoculated at an initioal OD600 of ˜0.3 with freshly harvested cells of strain BRKY-399 grown in aerobic pre-culture. The bioreactor temperature was maintained at 30° C. The fermentation was conducted in two phases: one phase for aerobic production of biomass and a second microaerobic phase for product formation (synthetic air was supplied in the headspace but not sparged in the medium). During the first phase, aerobic conditions were maintained by sparging with synthetic air at a rate of 0.1 L/min and agitation speed of 150 rpm. Initial pH was 5.8 and was allowed to drop to a level of 3.5 and then maintained at 3.5 by adding automatically a 1 M NaOH solution. Once glucose and acetate were exhausted and the OD600 reached a value >10 (˜48 h), a second pulse of 8 g/L glucose and 0.2 g/L acetate was injected in the bioreactor and the synthetic air sparging shifted to headspace and the agitation speed was increased to 450 rpm. This second phase was allowed to continue for ˜160 h. Potassium acetate was supplied at a concentration of 0.2 g/L whenever needed.

Sampling was performed daily. Cell biomass was calculated by measuring the absorbance at 600 nm in a ULTROSPEC 2000 spectrophotometer UV/visible (Pharmacia Biotech) after appropriate dilution in saline. For HPLC-RI and HPLC-UV analyses, the samples were filtered through a 0.2 μm filter (Millipore). Pyruvic, lactic and acetic acids, ethanol, glycerol, 2-propanol, 1,2-propanediol and sugars were separated and quantified by high-performance liquid chromatography (Waters 600 Chromatograph), using an ion exclusion column Aminex HPX-87H (Bio-Rad) and the IR and UV detectors in series. Operating conditions were: 0.04 mol L⁻¹ H₂SO₄ degassed eluent, flow rate 0.6 mL min⁻¹, column temperature 35° C. and refractometer temperature 50° C.

FIG. 7 show an exemplary carbon source consumption and metabolite profile of the two-phase fermentation of strain BRKY-399. Under the conditions provided it was possible to co-produce 2-propanol (200 mg/L) and 1,2-Propanediol (30 mg/L) after 160 h of culture from Glucose and sodium acetate (Total product yield=2.45 g per 100 g of substrates consumed). Three independent experiments were conducted and yielded similar results with regards to total product yield, although the relative amounts of 2-propanol and 1,2-PDO varied according to the specific microaerobic conditions.

This example shows that it is possible to use the ethanol-null yeast chassis for the co-production of bulk chemicals.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

1. A non-naturally occurring microorganism comprising:
a disruption of one or more enzymes that decarboxylate pyruvate and/or a disruption of one or more transcription factors of one or more enzymes that decarboxylate pyruvate;
a genetic modification that substantially decreases glucose import into the microorganism;
one or more polynucleotides encoding one or more enzymes in a pathway that produces cytosolic acetyl-CoA;
one or more polynucleotides encoding one or more enzymes in a pathway that catalyze a conversion of cytosolic acetyl-CoA to 2-propanol; and
one or more polynucleotides encoding one or more enzymes in a pathway that catalyze a conversion of dihydroxyacetone-phosphate to 1-propanol and/or 1,2-propanediol.)

2. The non-naturally occurring microorganism of claim 1, wherein the disruption in the one or more enzymes that decarboxylate pyruvate is a deletion or a mutation.)

3. The non-naturally occurring microorganism of claim 1, wherein the one or more enzymes that decarboxylate pyruvate include pdc1, pdc 5, and/or pdc6, and wherein the one or more transcription factors of the one or more enzymes that decarboxylate pyruvate include pdc2.)

4. The non-naturally occurring microorganism of claim 1, wherein the microorganism comprises an exogenous polynucleotide that encodes a transcription factor involved in glucose import.)

5. The non-naturally occurring microorganism of claim 1, wherein the microorganism comprises a genetic modification in an endogenous polynucleotide that encodes a transcription factor involved in glucose import.)

6. The non-naturally occurring microorganism of claim 5, wherein the genetic modification is a truncation of the MTH1 transcription factor.)

7. The non-naturally occurring microorganism of claim 1, wherein the one or more exogenous polynucleotides encoding one or more enzymes in a pathway that produces acetyl-CoA encode i.) pyruvate formate lyase and pyruvate formate lyase activating enzyme, ii) pyruvate dehydrogenase, dihydrolipoyl transacetylase and dihydrolipoamide dehydrogenase, iii) pyruvate dehydrogenase, dihydrolipoyl transacetylase, dihydrolipoamide dehydrogenase, and pyruvate dehydrogenase complex protein X, or any combination thereof.)

8. The non-naturally occurring microorganism of claim 1, wherein the microorganism is a eukaryote selected from the group consisting of: yeast, filamentous fungi, protozoa, and algae.)

9. The non-naturally occurring microorganism of claim 1, wherein the one or more polynucleotides coding for enzymes in a pathway that catalyzes a conversion of acetyl-CoA to 2-propanol include: one or more polynucleotides coding for enzymes in a pathway that catalyze a conversion of acetyl-CoA to acetoacetyl-CoA, one or more polynucleotides coding for enzymes in a pathway that catalyze a conversion of acetoacetyl-CoA to acetoacetate, one or more polynucleotides coding for enzymes in a pathway that catalyze a conversion of acetoacetate to acetone, and/or one or more polynucleotides coding for enzymes in a pathway that catalyze a conversion of acetone to 2-propanol.)

10. The non-naturally occurring microorganism of claim 1, wherein the one or more polynucleotides coding for enzymes in a pathway that catalyzes a conversion of dihydroxyacetone-phosphate to 1-propanol and/or 1,2-propanediol include: one or more polynucleotides coding for enzymes in a pathway that catalyze a conversion of dihydroxyacetone-phosphate to methylglyoxal, one or more polynucleotides coding for enzymes in a pathway that catalyze a conversion of methylglyoxal to hydroxyacetone, one or more polynucleotides coding for enzymes in a pathway that catalyze a conversion of hydroxyacetone to 1,2-propanediol, one or more polynucleotides coding for enzymes in a pathway that catalyze a conversion of methylglyoxal to lactaldehyde, one or more polynucleotides coding for enzymes in a pathway that catalyze a conversion of lactaldehyde to 1,2-propanediol, one or more polynucleotides coding for enzymes in a pathway that catalyze a conversion of 1,2-propanediol to propionaldehyde, and/or one or more polynucleotides coding for enzymes in a pathway that catalyze a conversion of propionaldehyde to 1-propanol.)

11. A non-naturally occurring microorganism comprising:
one or more exogenous polynucleotides encoding one or more enzymes in a pathway that produces cytosolic acetyl-CoA;
one or more polynucleotides coding for enzymes that catalyze a conversion of cytosolic acetyl-CoA to 2-propanol; and
one or more polynucleotides coding for enzymes in a pathway that catalyze a conversion of dihydroxyacetone-phosphate to 1-propanol and/or 1,2-propanediol,
wherein the microorganism has reduced levels of pyruvate decarboxylase enzymatic activity, and wherein the microorganism is capable of growing on a C6 sugar as a sole carbon source under anaerobic conditions.)

12. The non-naturally occurring microorganism of claim 11, wherein the microorganism has a disruption in one or more polynucleotides that code for one or more enzymes that decarboxylate pyruvate or a disruption in one or more polynucleotides that code for a transcription factor of an enzyme that decarboxylates pyruvate.)

13. The non-naturally occurring microorganism of claim 11, wherein the disruption in the one or more enzymes that decarboxylate pyruvate is a deletion or a mutation.)

14. The non-naturally occurring microorganism of claim 13, wherein the one or more enzymes that decarboxylate pyruvate include pdc1, pdc 5, and/or pdc6, and wherein the one or more transcription factors of the one or more enzymes that decarboxylate pyruvate include pdc2.)

15. The non-naturally occurring microorganism of claim 11, wherein the one or more polynucleotides coding for enzymes in a pathway that catalyzes a conversion of acetyl-CoA to 2-propanol include: one or more polynucleotides coding for enzymes in a pathway that catalyze a conversion of acetyl-CoA to acetoacetyl-CoA, one or more polynucleotides coding for enzymes in a pathway that catalyze a conversion of acetoacetyl-CoA to acetoacetate, one or more polynucleotides coding for enzymes in a pathway that catalyze a conversion of acetoacetate to acetone, and/or one or more polynucleotides coding for enzymes in a pathway that catalyze a conversion of acetone to 2-propanol.)

16. The non-naturally occurring microorganism of claim 11, wherein the one or more polynucleotides coding for enzymes in a pathway that catalyzes a conversion of dihydroxyacetone-phosphate to 1-propanol include and/or 1,2-propanediol: one or more polynucleotides coding for enzymes in a pathway that catalyze a conversion of dihydroxyacetone-phosphate to methylglyoxal, one or more polynucleotides coding for enzymes in a pathway that catalyze a conversion of methylglyoxal to hydroxyacetone, one or more polynucleotides coding for enzymes in a pathway that catalyze a conversion of hydroxyacetone to 1,2-propanediol, one or more polynucleotides coding for enzymes in a pathway that catalyze a conversion of methylglyoxal to lactaldehyde, one or more polynucleotides coding for enzymes in a pathway that catalyze a conversion of lactaldehyde to 1,2-propanediol, one or more polynucleotides coding for enzymes in a pathway that catalyze a conversion of 1,2-propanediol to propionaldehyde, and/or one or more polynucleotides coding for enzymes in a pathway that catalyze a conversion of propionaldehyde to 1-propanol.)

17. A method for co-producing 2-propanol with 1-propanol and/or 1,2-propanediol from a fermentable carbon source under anaerobic conditions, the method comprising:
a.) providing a fermentable carbon source;
b.) contacting the fermentable carbon source with the non-naturally occurring microorganism of claim 1 in a fermentation media under substantially anaerobic conditions, and
c.) expressing the polynucleotides in the microorganism for the co-production of 2-propanol with 1-propanol and/or 1,2-propanediol,
wherein the microorganism co-produces 2-propanol with 1-propanol and/or 1,2-propanediol.)

18. The method of claim 17, wherein the fermentable carbon source is sugarcane juice, sugarcane molasses, hydrolyzed starch, hydrolyzed lignocellulosic materials, glucose, sucrose, fructose, lactate, lactose, xylose, pyruvate, or glycerol in any form or mixture thereof.)

19. The method of claim 17, wherein the fermentable carbon source is a monosaccharide, oligosaccharide, or polysaccharide.)

20. The method of claim 17, wherein the produced 2-propanol with 1-propanol and/or 1,2-propanediol are secreted by the microorganism into the fermentation media.)

21. The method of claim 20 further comprising recovering the produced 2-propanol with 1-propanol and/or 1,2-propanediol from the fermentation media.
